(19) United States
(12) Patent Application Publication
(10) Pub. No.: US 2024/0352103 A1
(43) Pub. Date: Oct. 24, 2024

(54) ANTI-FEL D1 ANTIBODY FORMULATIONS

(71) Applicant: Regeneron Pharmaceuticals, Inc., Tarrytown, NY (US)

(72) Inventors: Hunter Chen, New York, NY (US); Shalini Minocha, Wilton, CT (US); Ritwik Nandagiri, Delmar, NY (US); Saurav Sharma, Danbury, CT (US); Xiaolin Tang, Old Tappan, NJ (US); Franco Tzul, Cortlandt Manor, NY (US)

(21) Appl. No.: 18/592,388

(22) Filed: Feb. 29, 2024

Related U.S. Application Data
(60) Provisional application No. 63/487,791, filed on Mar. 1, 2023.

(51) Int. Cl. C07K 16/18; A61K 39/395; A61K 47/18; A61K 47/22; A61K 47/26

(52) U.S. Cl. CPC C07K 16/18; A61K 39/39591; A61K 47/183; A61K 47/22; A61K 47/26

(57) ABSTRACT

The present disclosure provides pharmaceutical formulations comprising one or more antibodies or antigen-binding fragments thereof that bind to the cat allergen Fel d1. The formulations may contain, in addition to the anti-Fel d1 antibody, a buffer, a thermal stabilizer, a viscosity reducer, and a surfactant. In one aspect, co-formulations comprising two anti-Fel d1 antibodies are provided that have acceptable stability and optimal viscosity.

### CROSS-REFERENCE TO RELATED APPLICATIONS

This application claims priority to U.S. Provisional Patent Application No. 63/487,791, filed Mar. 1, 2023, the contents of which are incorporated by reference herein.

### SEQUENCE LISTING

An official copy of the sequence listing is submitted concurrently with the specification electronically via Patent Center. The contents of the electronic sequence listing (10852US01_Sequence_Listing_ST26.xml; Size: 32,768 bytes; and Date of Creation: Feb. 29, 2024) is herein incorporated by reference in its entirety.

### FIELD OF THE INVENTION

The present disclosure relates to therapeutic antibody formulations. More specifically, the present disclosure relates to the field of pharmaceutical formulations comprising one or more human antibodies that specifically bind to the cat allergen Fel d1.

### BACKGROUND

Cat allergens are among the most significant indoor allergens and a common cause of IgE-mediated allergic disease worldwide. The most dominant cat allergen is Felis domesticus allergen 1 (Fel d1); it is estimated that about 90% to 95% of cat allergic individuals are sensitized to Fel d1 (van Ree et al., J. Allergy Clin. Immunol. 1999, 104:1223-1230). Fel d1 is produced by the skin and by salivary and lacrimal glands of the cat (Kleine-Tebbe et al., Int Arch Allergy Immunol 1993, 100:256-62). Dried saliva and dandruff are spread from cat hair into the surrounding environment as small airborne particles that readily adhere to surfaces such as walls, carpets, and furniture. While the highest amount of Fel d1 allergen is found in households with cats, the allergen can also be carried into locations without cats such as cars, schools, and homes and may persist in these areas for months to years (see, e.g., Neisler et al., Aerobiologia 2016, 32:571-580). Thus, Fel d1 is an attractive therapeutic target for the treatment of cat-allergic patients. Antibodies to Fel d1 are described in, for example, U.S. Pat. Nos. 9,079,948, 10,047,153, and 11,174,305.

Therapeutic macromolecules such as antibodies must be formulated in a manner that not only makes the molecules suitable for administration to patients, but also maintains their stability during storage. For example, therapeutic antibodies in liquid solution are prone to degradation, aggregation, and/or undesired chemical modifications unless the solution is formulated properly. The stability of an antibody in liquid formulation depends not only on the kinds of excipients used in the formulation, but also on the amounts and proportions of the excipients relative to one another. Furthermore, other considerations aside from stability must be taken into account when preparing a liquid antibody formulation. Examples of such additional considerations include the viscosity of the solution and the concentration of antibody that can be accommodated by a given formulation, and the visual quality or appeal of the formulation. The co-formulation of two or more therapeutic antibodies in the same formulation is even more challenging, as each antibody may have very different requirements for suitable pH, excipients, and/or ionic strength.

### SUMMARY

In one aspect, stable liquid pharmaceutical formulations comprising at least one antibody that specifically binds to Fel d1 are provided. In some embodiments, the formulation comprises:
(a) at least one antibody that specifically binds to Fel d1, wherein the antibody is selected from the group consisting of:
   (i) an antibody comprising an HCDR1 comprising the amino acid sequence of SEQ ID NO:4, an HCDR2 comprising the amino acid sequence of SEQ ID NO:6, an HCDR3 comprising the amino acid sequence of SEQ ID NO:8, an LCDR1 comprising the amino acid sequence of SEQ ID NO:12, an LCDR2 comprising the amino acid sequence AAS, and an LCDR3 comprising the amino acid sequence of SEQ ID NO:14; and
   (ii) an antibody comprising an HCDR1 comprising the amino acid sequence of SEQ ID NO:18, an HCDR2 comprising the amino acid sequence of SEQ ID NO:20, an HCDR3 comprising the amino acid sequence of SEQ ID NO:22, an LCDR1 comprising the amino acid sequence of SEQ ID NO:26, an LCDR2 comprising the amino acid sequence KAS, and an LCDR3 comprising the amino acid sequence of SEQ ID NO:28;
(b) a buffer comprising histidine;
(c) a thermal stabilizer; and
(d) a surfactant;
wherein the formulation has a pH of 6.0±0.3.

In some embodiments, the stable liquid pharmaceutical formulation comprises (i) a first antibody that specifically binds to Fel d1, wherein the first antibody comprises an HCDR1 comprising the amino acid sequence of SEQ ID NO:4, an HCDR2 comprising the amino acid sequence of SEQ ID NO:6, an HCDR3 comprising the amino acid sequence of SEQ ID NO:8, an LCDR1 comprising the amino acid sequence of SEQ ID NO:12, an LCDR2 comprising the amino acid sequence AAS, and an LCDR3 comprising the amino acid sequence of SEQ ID NO:14; and (ii) a second antibody that specifically binds to Fel d1, wherein the second antibody comprises an HCDR1 comprising the amino acid sequence of SEQ ID NO:18, an HCDR2 comprising the amino acid sequence of SEQ ID NO:20, an HCDR3 comprising the amino acid sequence of SEQ ID NO:22, an LCDR1 comprising the amino acid sequence of SEQ ID NO:26, an LCDR2 comprising the amino acid sequence KAS, and an LCDR3 comprising the amino acid sequence of SEQ ID NO:28.

In some embodiments, the first antibody comprises a heavy chain variable region (HCVR) comprising the amino acid sequence of SEQ ID NO:2 and a light chain variable region (LCVR) comprising the amino acid sequence of SEQ ID NO:10.

In some embodiments, the second antibody comprises an HCVR comprising the amino acid sequence of SEQ ID NO:16 and an LCVR comprising the amino acid sequence of SEQ ID NO:24.

In some embodiments, the total antibody concentration is up to 200 mg/mL (e.g., up to 50 mg/mL, up to 100 mg/mL, or up to 150 mg/mL). In some embodiments, the total antibody concentration is from 5 mg/mL to 200 mg/mL. In some embodiments, the total antibody concentration is from 50 mg/mL to 200 mg/mL. In some embodiments, the total antibody concentration is 150 mg/mL±15 mg/mL. In some embodiments, wherein the formulation comprises two antibodies, the first antibody and the second antibody are present in the formulation at a molecular ratio of 1:1 or of about 1:1. In some embodiments, the first antibody is present at a concentration of 75 mg/mL±7.5 mg/mL and the second antibody is present at a concentration of 75 mg/mL±7.5 mg/mL.

In some embodiments, the buffer comprises histidine at a concentration of from 5 mM to 25 mM.

In some embodiments, the histidine is present at a concentration of 10 mM±2 mM.

In some embodiments, the thermal stabilizer comprises sucrose, trehalose, proline, or sorbitol.

In some embodiments, the thermal stabilizer comprises sucrose at a concentration of from 2.5% w/v to 7.5% w/v. In some embodiments, the sucrose is present at a concentration of 5% w/v±1% w/v.

In some embodiments, the stable liquid pharmaceutical formulation further comprises a viscosity reducer.

In some embodiments, the viscosity reducer comprises arginine. In some embodiments, the arginine is present at a concentration of 60 mM to 130 mM. In some embodiments, the arginine is present at a concentration of at least 70 mM. In some embodiments, the arginine is present at a concentration of 70 mM±14 mM.

In some embodiments, the surfactant comprises polysorbate 20 or polysorbate 80. In some embodiments, the polysorbate 20 or polysorbate 80 is present at a concentration of from 0.05% w/v to 0.25% w/v, e.g., from 0.05% w/v to 0.2% w/v or from 0.075% w/v to 0.2% w/v. In some embodiments, the surfactant comprises polysorbate 80 at a concentration of 0.15% w/v±0.075% w/v.

In some embodiments, the stable liquid pharmaceutical formulation comprises:
histidine at a concentration of 10 mM±2 mM;
sucrose at a concentration of 5% w/v±1% w/v;
arginine at a concentration of 70 mM±14 mM;
polysorbate 80 at a concentration of 0.15% w/v±0.075% w/v;
wherein the total antibody concentration in the formulation is 150 mg/mL±15 mg/mL, and wherein the formulation has a pH 6.0±0.3.

In some embodiments, the stable liquid pharmaceutical formulation has a viscosity ≤30 cP at 20° C. In some embodiments, the stable liquid pharmaceutical formulation has a viscosity ≤25 cP at 20° C. In some embodiments, the stable liquid pharmaceutical formulation has a viscosity ≤20 cP at 20° C. In some embodiments, the stable liquid pharmaceutical formulation has a viscosity ≤15 cP at 20° C.

In some embodiments, the stable liquid pharmaceutical formulation comprises:
an anti-Fel D1 antibody that comprises an HCVR comprising the amino acid sequence of SEQ ID NO:2 and an LCVR comprising the amino acid sequence of SEQ ID NO:10, wherein the concentration of the antibody is from 50 mg/mL to 190 mg/mL;
histidine at a concentration of 10 mM±2 mM;
sucrose at a concentration of 5% w/v±1% w/v;
arginine at a concentration of 70 mM±14 mM; and
polysorbate 80 at a concentration of 0.15% w/v±0.075% w/v;
wherein the formulation has a pH 6.0±0.3.

In some embodiments, the stable liquid pharmaceutical formulation comprises:
an anti-Fel D1 antibody that comprises an HCVR comprising the amino acid sequence of SEQ ID NO:16 and an LCVR comprising the amino acid sequence of SEQ ID NO:24, wherein the concentration of the antibody is from 50 mg/mL to 190 mg/mL;
histidine at a concentration of 10 mM±2 mM;
sucrose at a concentration of 5% w/v±1% w/v; and
polysorbate 20 or polysorbate 80 at a concentration of 0.15% w/v±0.075% w/v;
wherein the formulation has a pH 6.0±0.3.

In some embodiments, the stable liquid pharmaceutical formulation further comprises a viscosity reducer. In some embodiments, the viscosity reducer is arginine at a concentration of 70 mM±14 mM.

In some embodiments, the stable liquid pharmaceutical formulation comprises:
a first antibody that specifically binds to Fel d1, wherein the first antibody comprises an HCVR comprising the amino acid sequence of SEQ ID NO:2 and an LCVR comprising the amino acid sequence of SEQ ID NO:10, wherein the concentration of the first antibody is 75 mg/mL±10 mg/mL;
a second antibody that specifically binds to Fel d1, wherein the second antibody comprises an HCVR comprising the amino acid sequence of SEQ ID NO:16 and an LCVR comprising the amino acid sequence of SEQ ID NO:24, wherein the concentration of the second antibody is 75 mg/mL±10 mg/mL;
histidine at a concentration of 10 mM±2 mM;
sucrose at a concentration of 5% w/v±1% w/v;
arginine at a concentration of 70 mM±14 mM; and
polysorbate 80 at a concentration of 0.15% w/v±0.075% w/v;
wherein the formulation has a pH 6.0±0.3.

In some embodiments, the stable liquid pharmaceutical formulation is contained in a container that is selected from the group consisting of a glass vial, a syringe, a pre-filled syringe, a pen delivery device, and an autoinjector delivery device. In some embodiments, the stable liquid pharmaceutical formulation is contained in a large volume device or bolus injector.

In another aspect, a container containing a stable liquid pharmaceutical formulation as described herein is provided.

In yet another aspect, a kit is provided. In some embodiments, the kit comprises:
(i) a container containing a stable liquid pharmaceutical formulation as described herein; and
(ii) labeling for use of the pharmaceutical formulation. In some embodiments, the labeling recites subcutaneous or intravenous administration of the pharmaceutical formulation.

In some embodiments, the kit comprises a stable liquid pharmaceutical formulation comprising:
(a) an antibody that specifically binds to Fel d1, wherein the antibody comprises an HCDR1 comprising the amino acid sequence of SEQ ID NO:4, an HCDR2 comprising the amino acid sequence of SEQ ID NO:6, an HCDR3 comprising the amino acid sequence of SEQ ID NO:8, an LCDR1 comprising the amino acid sequence of SEQ ID NO:12, an LCDR2 comprising the amino acid sequence AAS, and an LCDR3 comprising the amino acid sequence of SEQ ID NO:14;
(b) a buffer comprising histidine;
(c) a thermal stabilizer; and
(d) a surfactant;
wherein the formulation has a pH of 6.0±0.3.

In some embodiments, the kit comprises an antibody comprising an HCVR comprising the amino acid sequence of SEQ ID NO:2 and an LCVR comprising the amino acid sequence of SEQ ID NO:10.

In some embodiments, the kit comprises a stable liquid pharmaceutical formulation comprising:
an anti-Fel D1 antibody that comprises an HCVR comprising the amino acid sequence of SEQ ID NO:2 and an LCVR comprising the amino acid sequence of SEQ ID NO:10, wherein the concentration of the antibody is from 50 mg/mL to 190 mg/mL;
histidine at a concentration of 10 mM±2 mM;
sucrose at a concentration of 5% w/v±1% w/v; and
polysorbate 80 at a concentration of 0.15% w/v±0.075% w/v;
wherein the formulation has a pH 6.0±0.3.

In some embodiments, the kit comprises a stable liquid pharmaceutical formulation comprising:
(a) an antibody that specifically binds to Fel d1, wherein the antibody comprises an HCDR1 comprising the amino acid sequence of SEQ ID NO:18, an HCDR2 comprising the amino acid sequence of SEQ ID NO:20, an HCDR3 comprising the amino acid sequence of SEQ ID NO:22, an LCDR1 comprising the amino acid sequence of SEQ ID NO:26, an LCDR2 comprising the amino acid sequence KAS, and an LCDR3 comprising the amino acid sequence of SEQ ID NO:28;
(b) a buffer comprising histidine;
(c) a thermal stabilizer; and
(d) a surfactant;
wherein the formulation has a pH of 6.0±0.3.

In some embodiments, the kit comprises an antibody comprising an HCVR comprising the amino acid sequence of SEQ ID NO:16 and an LCVR comprising the amino acid sequence of SEQ ID NO:24.

In some embodiments, the kit comprises a stable liquid pharmaceutical formulation comprising:
an anti-Fel D1 antibody that comprises an HCVR comprising the amino acid sequence of SEQ ID NO:16 and an LCVR comprising the amino acid sequence of SEQ ID NO: 24, wherein the concentration of the antibody is from 50 mg/mL to 190 mg/mL;
histidine at a concentration of 10 mM±2 mM;
sucrose at a concentration of 5% w/v±1% w/v; and
polysorbate 20 or polysorbate 80 at a concentration of 0.15% w/v±0.075% w/v;
wherein the formulation has a pH 6.0±0.3.

In some embodiments, the kit comprises a stable liquid pharmaceutical formulation as described above, and further comprising a viscosity reducer. In some embodiments, the viscosity reducer is arginine at a concentration of 70 mM±14 mM.

In some embodiments, the kit comprises a stable liquid pharmaceutical formulation comprising:
a first antibody that specifically binds to Fel d1, wherein the first antibody comprises an HCVR comprising the amino acid sequence of SEQ ID NO:2 and an LCVR comprising the amino acid sequence of SEQ ID NO:10, wherein the concentration of the first antibody is 75 mg/mL±10 mg/mL;
a second antibody that specifically binds to Fel d1, wherein the second antibody comprises an HCVR comprising the amino acid sequence of SEQ ID NO:16 and an LCVR comprising the amino acid sequence of SEQ ID NO:24, wherein the concentration of the second antibody is 75 mg/mL±10 mg/mL;
histidine at a concentration of 10 mM±2 mM;
sucrose at a concentration of 5% w/v±1% w/v;
arginine at a concentration of 70 mM±14 mM; and
polysorbate 80 at a concentration of 0.15% w/v±0.075% w/v;
wherein the formulation has a pH 6.0±0.3.

Other embodiments will be apparent from a review of the ensuing detailed description.

### BRIEF DESCRIPTION OF THE FIGURES

FIG. 1A. Percentage increase in HMW species of formulations of 150 mg/mL REGN1908 (▪) or REGN1909 (♦) in 10 mM histidine buffer at different pHs after incubation at 45° C. for 28 days.

FIG. 1B. Percentage increase in HMW species of formulations of 150 mg/mL REGN1908 (▪) or REGN1909 (♦) in 10 mM histidine buffer at different pHs after incubation at 25° C. for 2 months.

FIG. 2. Viscosity (cP at 20° C.) of formulations of 150 mg/mL REGN1908 (▪) or REGN1909 (•) in 10 mM histidine buffer at different pHs.

FIG. 3. Viscosity (cP at 20° C.) of formulations comprising 10 mM histidine, pH 6.0, and REGN1908 (▪) or REGN1909 (•) at different concentrations.

FIG. 4. Effect of excipients on viscosity (cP at 20° C.) for formulations comprising a target concentration of 150 mg/mL REGN1908 or REGN1909, and either 10 mM histidine pH 6.0, 10 mM histidine pH 6.0+70 mM arginine, 100 mM histidine pH 6.0, 10 mM histidine pH 6.0+3% proline, or 10 mM histidine pH 6.0+70 mM NaCl.

FIG. 5. Effect of the cryoprotectant sucrose on viscosity (cP at 20° C.) for formulations comprising a target concentration of 150 mg/mL REGN1908 (♦) or REGN1909 (▪), 10 mM histidine, pH 6.0.

FIG. 6. Effect of viscosity reducer and cryoprotectant on viscosity (cP at 20° C.) for formulation comprising a target concentration of 150 mg/mL REGN1908, 10 mM, pH 6.2, in the absence of viscosity reducer and cryoprotectant or in the presence of 70 mM arginine+5% sucrose.

FIG. 7A. Mean viscosity (mPa-s/cP) of formulations containing a target concentration of 150 mg/mL REGN1908, 10 mM histidine, 5% sucrose, pH 6.1 with different amounts of viscosity reducer arginine.

FIG. 7B. Mean viscosity (mPa-s/cP) of formulations containing a target concentration of 150 mg/mL REGN1909, 10 mM histidine, 5% sucrose, pH 6.1 with different amounts of viscosity reducer arginine.

FIG. 7C. Mean viscosity (mPa-s/cP) of formulations containing a target concentration of 150 mg/mL total antibody (REGN1908+REGN1909), 10 mM histidine, 5% sucrose, pH 6.1 with different amounts of viscosity reducer arginine.

FIG. 8A. REGN1908-REGN1909 co-formulations without polysorbate 20 (PS20) or with 0.01%, 0.025%, 0.05%, 0.075%, 0.1%, or 0.2% (w/v) PS20 were evaluated in an agitation assay for aggregation suppression, measured by percent change in HMW at time 0 (•), 24 hours (▴), or 48 hours (▾). Inset graph=a zoomed/exploded view of the data, enabling visualization of the minimum amount of PS20 surfactant required to effect little to no change in % HMW species (aggregates). LSL=Lower Specification Limit.

FIG. 8B. REGN1908-REGN1909 co-formulations without polysorbate 80 (PS80) or with 0.01%, 0.025%, 0.05%, 0.075%, 0.1%, or 0.2% (w/v) PS80 were evaluated in an agitation assay for aggregation suppression, measured by percent change in HMW at time 0 (•), 24 hours (▴), or 48 hours (▾). Inset graph=a zoomed/exploded view of the data, enabling visualization of the minimum amount of PS80 surfactant required to effect little to no change in % HMW species (aggregates). LSL=Lower Specification Limit.

FIG. 9. Subvisible particle formation was evaluated using micro-fluid imaging (>10 μM and >25 μM) at time 0 or 48 hours, for REGN1908-REGN1909 co-formulations without polysorbate 20 (PS20) or polysorbate 80 (PS80) or with 0.01%, 0.025%, 0.05%, 0.075%, 0.1%, or 0.2% (w/v) PS20 or PS80.

FIG. 10A. Accelerated stability study for co-formulations comprising REGN1908-REGN1909 (150 mg/mL total antibody), 10 mM histidine, 70 mM arginine, 0.15% (w/v) polysorbate 20, pH 6.0 and either 2.5% sorbitol (•), 3% proline (▴), 5% sucrose (▾), 5% trehalose (▪), or no cryoprotectant (♦).

FIG. 10B. Freeze/thaw stability study for co-formulations comprising REGN1908-REGN1909 (150 mg/mL total antibody), 10 mM histidine, 70 mM arginine, 0.15% (w/v) polysorbate 20, pH 6.0 and either 2.5% sorbitol (•), 3% proline (▴), 5% sucrose (▾), 5% trehalose (▪), or no cryoprotectant (♦).

FIG. 10C. Accelerated stability study for co-formulations comprising REGN1908-REGN1909 (150 mg/mL total antibody), 10 mM histidine, 70 mM arginine, 0.15% (w/v) polysorbate 80, pH 6.0 and either 2.5% sorbitol (•), 3% proline (▴), 5% sucrose (▾), 5% trehalose (▪), or no cryoprotectant (♦).

FIG. 10D. Freeze/thaw stability study for co-formulations comprising REGN1908-REGN1909 (150 mg/mL total antibody), 10 mM histidine, 70 mM arginine, 0.15% (w/v) polysorbate 80, pH 6.0 and either 2.5% sorbitol (•), 3% proline (▴), 5% sucrose (▾), 5% trehalose (▪), or no cryoprotectant (♦).

FIG. 11. Thermal profiler demonstrates predicted formulation responses at 40° C. as a function of ranging formulation components. Columns depict formulation components: REGN1908-REGN1909 concentration (from 135 mg/mL to 165 mg/mL), pH (from 5.5 to 6.5), histidine (from 6 mM to 14 mM), polysorbate 80 (from 0.075% to 0.2% w/v), arginine (from 42 mM to 98 mM), and sucrose (from 3% to 7% w/v). Rows depict a formulation attribute's predicted responses: change in percent total protein recovery, change in percent total HMW, change in percent total acidic species, change in percent total basic species, change in particulate formation (10-micron bin), and change in particulate formation (25-micron bin).

FIG. 12. Agitation profiler demonstrates predicted formulation responses under agitation stress as a function of ranging formulation components. Columns depict formulation components: REGN1908-REGN1909 concentration (from 135 mg/mL to 165 mg/mL), pH (from 5.5 to 6.5), histidine (from 6 mM to 14 mM), polysorbate 80 (from 0.075% to 0.2% w/v), arginine (from 42 mM to 98 mM), and sucrose (from 3% to 7% w/v). Rows depict a formulation attribute's predicted responses: change in percent total protein recovery, change in percent total HMW, change in particulate formation (10-micron bin), and change in particulate formation (25-micron bin).

FIG. 13. Freeze-thaw profiler demonstrates predicted formulation responses under freeze-thaw stress as a function of ranging formulation components. Columns depict formulation components: REGN1908-REGN1909 concentration (from 135 mg/mL to 165 mg/mL), pH (from 5.5 to 6.5), histidine (from 6 mM to 14 mM), polysorbate 80 (from 0.075% to 0.2% w/v), arginine (from 42 mM to 98 mM), and sucrose (from 3% to 7% w/v). Rows depict a formulation attribute's predicted responses: change in percent total protein recovery, change in percent total HMW, change in particulate formation (10-micron bin), and change in particulate formation (25-micron bin).

FIG. 14. T=0 profiler demonstrates predicted viscosity and osmolality responses as a function of ranging formulation components. Columns depict formulation components: REGN1908-REGN1909 concentration (from 135 mg/mL to 165 mg/mL), pH (from 5.5 to 6.5), histidine (from 6 mM to 14 mM), polysorbate 80 (from 0.075% to 0.2% w/v), arginine (from 42 mM to 98 mM), and sucrose (from 3% to 7% w/v). Rows depict a formulation attribute's predicted responses: viscosity at t=0 and osmolality at t=0.

### DETAILED DESCRIPTION

### Definitions

Before the present invention is described, it is to be understood that the invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

As used herein, the expression “pharmaceutical formulation” means a combination of at least one active ingredient (e.g., an antibody, small molecule, compound, etc. which is capable of exerting a biological effect in a human or non-human animal), and at least one inactive ingredient which, when combined with the active ingredient and/or one or more additional inactive ingredients, is suitable for therapeutic administration to a human or non-human animal. The term “formulation,” as used herein, means “pharmaceutical formulation” unless specifically indicated otherwise. The present disclosure provides pharmaceutical formulations comprising at least one therapeutic polypeptide. According to certain embodiments of the present disclosure, the therapeutic polypeptide is an antibody, or an antigen-binding fragment thereof, that specifically binds to Fel d1. In some embodiments, the present disclosure includes pharmaceutical formulations that comprise: (i) one or more human antibodies that specifically bind to Fel d1; (ii) one or more buffers; (iii) a thermal stabilizer; (iv) a viscosity reducer; and optionally (v) a surfactant. In some embodiments, the present disclosure includes pharmaceutical formulations that comprise: (i) one or more human antibodies that specifically bind to Fel d1; (ii) one or more buffers; (iii) a thermal stabilizer; (iv) a surfactant; and optionally (v) a viscosity reducer. In one particular embodiment, the pharmaceutical formulation comprises: (i) two human antibodies that each specifically bind to Fel d1; (ii) one or more buffers; (iii) a thermal stabilizer; (iv) a viscosity reducer; and (v) a surfactant. Additional components may be included in the formulations of the present disclosure if such components do not significantly interfere with the viscosity and stability of the formulation. Specific exemplary components and formulations included within the present disclosure are described in detail below.

The term “excipient”, as used herein, means any non-therapeutic agent added to the formulation to provide a desired consistency, viscosity, or stabilizing effect.

The pharmaceutical formulations of the present disclosure may, in certain embodiments, be fluid formulations. As used herein, the expression “fluid formulation” means a mixture of at least two components that exists predominantly in the fluid state at about 2° C. to about 45° C. Fluid formulations include, inter alia, liquid formulations. Fluid formulations may be of low, moderate or high viscosity depending on their particular constituents.

The term “antibody,” as used herein, refers to an antigen-binding molecule or molecular complex comprising a set of complementarity determining regions (CDRs) that specifically bind to or interact with a particular antigen (e.g., Fel d1). The term “antibody,” as used herein, includes immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In some embodiments, the FRs of the antibody (or antigen-binding portion thereof) may be identical to the human germline sequences, or may be naturally or artificially modified. An amino acid consensus sequence may be defined based on a side-by-side analysis of two or more CDRs.

Unless specifically indicated otherwise, the term “antibody,” as used herein, also includes antigen-binding fragments of full antibody molecules. The terms “antigen-binding portion” of an antibody, “antigen-binding fragment” of an antibody, “antigen-binding domain,” and the like, as used herein, include any naturally occurring, enzymatically obtainable, synthetic, or genetically engineered polypeptide or glycoprotein that specifically binds an antigen to form a complex. Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein.

The term “human antibody,” as used herein, is intended to include antibodies having variable and constant regions derived from human germline immunoglobulin sequences. The human antibodies of the disclosure may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The term “recombinant antibody,” as used herein, is intended to include all antibodies that are prepared, expressed, created, or isolated by recombinant means. The term includes, but is not limited to, antibodies expressed using a recombinant expression vector transfected into a host cell (e.g., Chinese hamster ovary (CHO) cell) or cellular expression system, antibodies isolated from a recombinant, combinatorial human antibody library, and antibodies isolated from a non-human animal (e.g., a mouse, such as a mouse that is transgenic for human immunoglobulin genes (see e.g., Taylor et al. (1992) Nucl. Acids Res. 20:6287-6295). In some embodiments, the recombinant antibody is a recombinant human antibody. In some embodiments, recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

An “isolated antibody” refers to an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody.” An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The term “specifically binds,” or the like, means that an antibody or antigen-binding fragment thereof forms a complex with an antigen that is relatively stable under physiologic conditions. Specific binding can be characterized by an equilibrium dissociation constant of at least about 1×10⁻⁶ M or less, e.g., 10⁻⁷ M, 10⁻⁸ M, 10⁻⁹ M, 10⁻¹⁰ M, 10⁻¹¹ M, or 10⁻¹² M (a smaller K_{D} denotes a tighter binding). Methods for determining whether an antibody specifically binds to an antigen are known in the art and include, for example, equilibrium dialysis, surface plasmon resonance (e.g., BIACORE™), bio-layer interferometry assay (e.g., Octet® HTX biosensor), solution-affinity ELISA, and the like. In some embodiments, specific binding is measured in a surface plasmon resonance assay, e.g., at 25° C. or 37° C. An antibody or antigen-binding fragment that specifically binds an antigen from one species may or may not have cross-reactivity to other antigens, such as an orthologous antigen from another species.

### Anti-Fel d1 Antibodies and Antigen-Binding Fragments Thereof

The pharmaceutical formulations of the present disclosure may comprise a human antibody, or an antigen-binding fragment thereof, that specifically binds to the cat allergen Fel d1. In some embodiments, a pharmaceutical formulation of the disclosure comprises one or more (e.g., two, three, or more) antibodies that specifically bind to Fel d1. Antibodies to Fel d1 are described in, for example, U.S. Pat. Nos. 9,079,948, 10,047,153, and 11,174,305 and in WO 2013/166236, each of which is incorporated by reference herein.

In some embodiments, the pharmaceutical formulation comprises one or more antibodies, or antigen-binding fragments thereof, that specifically bind Fel d1, wherein the antibody comprises:
(a) a heavy chain complementarity determining region 1 (HCDR1) comprising an amino acid sequence selected from the group consisting of SEQ ID NOs:4 and 18;
(b) a heavy chain complementarity determining region 2 (HCDR2) comprising an amino acid sequence selected from the group consisting of SEQ ID NOs:6 and 20;
(c) a heavy chain complementarity determining region 3 (HCDR3) comprising an amino acid sequence selected from the group consisting of SEQ ID NOs:8 and 22;
(d) a light chain complementarity determining region 1 (LCDR1) comprising an amino acid sequence selected from the group consisting of SEQ ID NOs:12 and 26;
(e) a light chain complementarity determining region 2 (LCDR2) comprising an amino acid sequence selected from the group consisting of AAS and KAS; and
(f) a light chain complementarity determining region 3 (LCDR3) comprising an amino acid sequence selected from the group consisting of SEQ ID NOs:14 and 28.

In some embodiments, the anti-Fel d1 antibody comprises a heavy chain variable region (HCVR) comprising an amino acid sequence that has at least 85%, at least 90%, at least 91%, at least 92%, at least 93%, at least 94%, at least 95%, at least 96%, at least 97%, at least 98%, or at least 99% sequence identity to an amino acid sequence selected from the group consisting of SEQ ID NOs:2 and 16. In some embodiments, the anti-Fel d1 antibody comprises an HCVR comprising an amino acid sequence selected from the group consisting of SEQ ID NOs: 2 and 16.

In some embodiments, the anti-Fel d1 antibody comprises a light chain variable region (LCVR) comprising an amino acid sequence that has at least 85%, at least 90%, at least 91%, at least 92%, at least 93%, at least 94%, at least 95%, at least 96%, at least 97%, at least 98%, or at least 99% sequence identity to an amino acid sequence selected from the group consisting of SEQ ID NOs:10 and 24. In some embodiments, the anti-Fel d1 antibody comprises an LCVR comprising an amino acid sequence selected from the group consisting of SEQ ID NOs:10 and 24.

In some embodiments, a pharmaceutical formulation of the present disclosure comprises an anti-Fel d1 antibody or antigen-binding fragment thereof that comprises:
(a) an HCDR1 comprising the amino acid sequence of SEQ ID NO:4, an HCDR2 comprising the amino acid sequence of SEQ ID NO:6, and an HCDR3 comprising the amino acid sequence of SEQ ID NO:8, an LCDR1 comprising the amino acid sequence of SEQ ID NO:12, an LCDR2 comprising the amino acid sequence AAS, and an LCDR3 comprising the amino acid sequence of SEQ ID NO:14; or
(b) an HCDR1 comprising the amino acid sequence of SEQ ID NO:18, an HCDR2 comprising the amino acid sequence of SEQ ID NO:20, and an HCDR3 comprising the amino acid sequence of SEQ ID NO:22 an LCDR1 comprising the amino acid sequence of SEQ ID NO:26, an LCDR2 comprising the amino acid sequence KAS, and an LCDR3 comprising the amino acid sequence of SEQ ID NO:28.

In some embodiments, a pharmaceutical formulation of the present disclosure comprises at least two anti-Fel d1 antibodies or antigen-binding fragments thereof. In some embodiments, the pharmaceutical formulation comprises (i) a first antibody that specifically binds to Fel d1, wherein the first antibody comprises an HCDR1 comprising the amino acid sequence of SEQ ID NO:4, an HCDR2 comprising the amino acid sequence of SEQ ID NO:6, an HCDR3 comprising the amino acid sequence of SEQ ID NO:8, an LCDR1 comprising the amino acid sequence of SEQ ID NO:12, an LCDR2 comprising the amino acid sequence AAS, and an LCDR3 comprising the amino acid sequence of SEQ ID NO:14; and (ii) a second antibody that specifically binds to Fel d1, wherein the second antibody comprises an HCDR1 comprising the amino acid sequence of SEQ ID NO:18, an HCDR2 comprising the amino acid sequence of SEQ ID NO:20, an HCDR3 comprising the amino acid sequence of SEQ ID NO:22, an LCDR1 comprising the amino acid sequence of SEQ ID NO:26, an LCDR2 comprising the amino acid sequence KAS, and an LCDR3 comprising the amino acid sequence of SEQ ID NO:28.

In some embodiments, the anti-Fel d1 antibody comprises an HCDR1 comprising the amino acid sequence of SEQ ID NO:4, an HCDR2 comprising the amino acid sequence of SEQ ID NO:6, and an HCDR3 comprising the amino acid sequence of SEQ ID NO:8, an LCDR1 comprising the amino acid sequence of SEQ ID NO:12, an LCDR2 comprising the amino acid sequence AAS, and an LCDR3 comprising the amino acid sequence of SEQ ID NO:14.

In some embodiments, the anti-Fel d1 antibody comprises an HCVR comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO:2, and/or an LCVR comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO:10. In some embodiments, the anti-Fel d1 antibody comprises an HCVR comprising the amino acid sequence of SEQ ID NO:2, and/or an LCVR comprising the amino acid sequence of SEQ ID NO:10. In some embodiments, the anti-Fel d1 antibody comprises an HCVR consisting of the amino acid sequence of SEQ ID NO:2, and/or an LCVR consisting of the amino acid sequence of SEQ ID NO:10.

In some embodiments, the anti-Fel d1 antibody comprises a heavy chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO:29, and/or a light chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO:30. In some embodiments, the anti-Fel d1 antibody comprises a heavy chain comprising the amino acid sequence of SEQ ID NO:29, and/or a light chain comprising the amino acid sequence of SEQ ID NO:30. In some embodiments, the anti-Fel d1 antibody comprises a heavy chain consisting of the amino acid sequence of SEQ ID NO:29, and/or a light chain consisting of the amino acid sequence of SEQ ID NO:30.

In some embodiments, the anti-Fel d1 antibody comprises an HCDR1 comprising the amino acid sequence of SEQ ID NO:18, an HCDR2 comprising the amino acid sequence of SEQ ID NO:20, and an HCDR3 comprising the amino acid sequence of SEQ ID NO:22, an LCDR1 comprising the amino acid sequence of SEQ ID NO:26, an LCDR2 comprising the amino acid sequence KAS, and an LCDR3 comprising the amino acid sequence of SEQ ID NO:28.

In some embodiments, the anti-Fel d1 antibody comprises an HCVR comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO:16, and/or an LCVR comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO:24. In some embodiments, the anti-Fel d1 antibody comprises an HCVR comprising the amino acid sequence of SEQ ID NO:16, and/or an LCVR comprising the amino acid sequence of SEQ ID NO:24. In some embodiments, the anti-Fel d1 antibody comprises an HCVR consisting of the amino acid sequence of SEQ ID NO:16, and/or an LCVR consisting of the amino acid sequence of SEQ ID NO:24.

In some embodiments, the anti-Fel d1 antibody comprises a heavy chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO:31; and/or a light chain comprising an amino acid sequence that has at least 85%, at least 90%, at least 91%, at least 92%, at least 93%, at least 94%, at least 95%, at least 96%, at least 97%, at least 98%, or at least 99% sequence identity to SEQ ID NO:32. In some embodiments, the anti-Fel d1 antibody comprises a heavy chain comprising the amino acid sequence of SEQ ID NO:31, and/or a light chain comprising the amino acid sequence of SEQ ID NO:32. In some embodiments, the anti-Fel d1 antibody comprises a heavy chain consisting of the amino acid sequence of SEQ ID NO:31, and/or a light chain consisting of the amino acid sequence of SEQ ID NO:32.

In some embodiments, an anti-Fel d1 antibody comprises a Fc region selected from the group consisting of human IgG1, IgG2, IgG3, and IgG4 isotypes. In some embodiments, the anti-Fel d1 antibody comprises a human IgG4 isotype.

In some embodiments, an anti-Fel d1 antibody or antigen-binding fragment thereof comprises one or more amino acid substitutions, insertions, and/or deletions in the framework and/or CDR regions of the heavy and/or light chain variable domains as compared to the corresponding germline sequences from which the individual antibodies were derived. Such mutations can be readily ascertained by comparing the amino acid sequences disclosed herein to germ line sequences available from, for example, public antibody sequence databases. The antibodies of the present disclosure may comprise antigen binding fragments which are derived from any of the exemplary amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was originally derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germ line sequence from which the antibody was originally derived). Furthermore, the antibodies or antigen-binding fragments may contain any combination of two or more germline mutations within the framework and/or CDR regions, e.g., wherein certain individual residues are mutated to the corresponding residue of a particular germ line sequence while certain other residues that differ from the original germ line sequence are maintained or are mutated to the corresponding residue of a different germline sequence. Once obtained, antibodies or antigen-binding fragments that contain one or more germline mutations can be easily tested for one or more desired property such as, improved binding specificity, increased binding affinity, improved or enhanced antagonistic or agonistic biological properties, reduced immunogenicity, etc. Antibodies and antigen-binding fragments obtained in this general manner are encompassed within the present disclosure.

The present disclosure also includes antibodies or antigen-binding fragments that comprise variants of any of the HCVR, LCVR, and/or CDR amino acid sequences disclosed herein having one or more conservative substitutions. For example, the present disclosure includes antibodies or antigen-binding fragments comprising HCVR, LCVR, and/or CDR amino acid sequences with, e.g., 10 or fewer, 9 or fewer, 8 or fewer, 7 or fewer, 6 or fewer, 5 or fewer, 4 or fewer, 3 or fewer, 2 or fewer, or 1 conservative amino acid substitution(s) relative to any of the HCVR, LCVR, and/or CDR amino acid sequences disclosed herein. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-1445. A “moderately conservative” replacement is any change having a nonnegative value in the PAM250 log-likelihood matrix.

The present disclosure also includes antibodies or antigen-binding fragments comprising an HCVR, LCVR, and/or CDR amino acid sequence that is substantially identical to any of the HCVR, LCVR, and/or CDR amino acid sequences disclosed herein. In some embodiments, an antigen-binding molecule comprises HCVR, LCVR, and/or CDR amino acid sequence having at least 85% sequence identity, e.g., at least 90%, at least 91%, at least 92%, at least 93%, at least 94%, at least 95%, at least 96%, at least 97%, at least 98% or at least 99% sequence identity, to a sequence disclosed in Table 1. In some embodiments, an antigen-binding molecule comprises HCVR, LCVR, and/or CDR amino acid sequence having at least 85% sequence identity, e.g., at least 90%, at least 91%, at least 92%, at least 93%, at least 94%, at least 95%, at least 96%, at least 97%, at least 98% or at least 99% sequence identity, to a sequence disclosed in Table 1, wherein the differences in the amino acid residue(s) relative to the sequence disclosed in Table 1 are conservative substitutions or moderately conservative substitutions.

In some embodiments, the antibody or antigen-binding fragment thereof is chimeric, humanized, or fully human. In some embodiments, the antibody or antigen-binding fragment thereof is humanized. In some embodiments, the antibody or antigen-binding fragment thereof is fully human.

The amount of antibody or antigen-binding fragment thereof (or in the case of two or more antibodies, the amount of each antibody or the total amount of antibody) contained within the pharmaceutical formulations of the present disclosure may vary depending on the specific properties desired of the formulations, as well as the particular circumstances and purposes for which the formulations are intended to be used. In certain embodiments, the pharmaceutical formulations may contain about 1 mg/mL to about 500 mg/mL of total antibody; about 5 mg/mL to about 250 mg/mL of total antibody; about 5 mg/mL to about 200 mg/mL of total antibody; about 15 mg/mL to about 250 mg/mL of total antibody; about 25 mg/mL to about 200 mg/mL of total antibody; about 50 mg/mL to about 200 mg/mL of total antibody; about 100 mg/mL to about 200 mg/mL of total antibody; about 125 mg/mL to about 175 mg/mL of total antibody; or about 150 mg/mL to about 200 mg/mL of total antibody. For example, the formulations of the present disclosure can comprise about 5 mg/mL; about 10 mg/mL; about 15 mg/mL; about 20 mg/mL; about 25 mg/mL; about 30 mg/mL; about 35 mg/mL; about 40 mg/mL; about 45 mg/mL; about 50 mg/mL; about 55 mg/mL; about 60 mg/mL; about 65 mg/mL; about 70 mg/mL; about 75 mg/mL; about 80 mg/mL; about 85 mg/mL; about 90 mg/mL; about 95 mg/mL; about 100 mg/mL; about 105 mg/mL; about 110 mg/mL; about 115 mg/mL; about 120 mg/mL; about 125 mg/mL; about 130 mg/mL; about 135 mg/mL; about 140 mg/mL; about 145 mg/mL; about 150 mg/mL; about 155 mg/mL; about 160 mg/mL; about 165 mg/mL; about 170 mg/mL; about 175 mg/mL; about 180 mg/mL; about 185 mg/mL; about 190 mg/mL; about 195 mg/mL; about 200 mg/mL; about 205 mg/mL; about 210 mg/mL; about 215 mg/mL; about 220 mg/mL; about 225 mg/mL; about 230 mg/mL; about 235 mg/mL; about 240 mg/mL; about 245 mg/mL; or about 250 mg/mL total antibody or antigen-binding fragment(s) thereof, that bind specifically to Fel d1. In certain embodiments, the pharmaceutical formulations contain about 50±5 mg/mL of total antibody, about 75±7.5 mg/mL of total antibody, about 100±10 mg/mL of total antibody, about 120±12 mg/mL of total antibody, about 140±14 mg/mL of total antibody, about 150±15 mg/mL of total antibody, about 170±17 mg/mL of total antibody, or about 190±19 mg/mL of total antibody. In certain embodiments, the pharmaceutical formulations may contain up to 100 mg/mL, up to 150 mg/mL, up to 200 mg/mL, up to 250 mg/mL, up to 300 mg/mL, up to 400 mg/mL, or up to 500 mg/mL of total antibody.

For formulations comprising two or more anti-Fel d1 antibodies, in some embodiments each antibody is present in an amount from about 1 mg/mL to about 250 mg/mL, e.g., about 5 mg/mL to about 200 mg/mL, about 10 mg/mL to about 150 mg/mL, about 20 mg/mL to about 100 mg/mL, about 50 mg/mL to about 150 mg/mL, or about 50 mg/mL to about 100 mg/mL. In some embodiments, each antibody is present in an amount of about 1 mg/mL; about 2 mg/mL; about 5 mg/mL; about 10 mg/mL; about 15 mg/mL; about 20 mg/mL; about 25 mg/mL; about 30 mg/mL; about 35 mg/mL; about 40 mg/mL; about 45 mg/mL; about 50 mg/mL; about 55 mg/mL; about 60 mg/mL; about 65 mg/mL; about 70 mg/mL; about 75 mg/mL; about 80 mg/mL; about 85 mg/mL; about 90 mg/mL; about 95 mg/mL; about 100 mg/mL; about 105 mg/mL; about 110 mg/mL; about 115 mg/mL; about 120 mg/mL; about 125 mg/mL; about 130 mg/mL; about 135 mg/mL; about 140 mg/mL; about 145 mg/mL; about 150 mg/mL; about 155 mg/mL; about 160 mg/mL; about 165 mg/mL; about 170 mg/mL; about 175 mg/mL; about 180 mg/mL; about 185 mg/mL; about 190 mg/mL; about 195 mg/mL; or about 200 mg/mL. In certain embodiments, the pharmaceutical formulations contain about 25±3 mg/mL of each antibody, about 50±5 mg/mL of each antibody, about 75±7.5 mg/mL of each antibody, or about 100±10 mg/mL of each antibody. In some embodiments, each antibody is present in the formulation in the same amount. In some embodiments, the antibodies are present in the formulation in different amounts. In some embodiments, the antibodies (e.g., REGN1908 and REGN1909) are present in a molecular ratio of 1:1 or a molecular ratio of about 1:1. In some embodiments, the antibodies (e.g., REGN1908 and REGN1909) are present in a molecular ratio of about 1.5:1 or about 1:1.5. In some embodiments, the antibodies (e.g., REGN1908 and REGN1909) are present in a molecular ratio of about 2:1 or about 1:2.

### Bioequivalents

The present disclosure encompasses formulations comprising antibodies having amino acid sequences that vary from those of the described antibodies but that retain the ability to bind Fel d1. Such variant molecules comprise one or more additions, deletions, or substitutions of amino acids when compared to a parent sequence, but exhibit biological activity that is essentially equivalent to that of the described antibodies. Likewise, the nucleic acid sequences encoding the antibodies of the present disclosure encompass sequences that comprise one or more additions, deletions, or substitutions of nucleotides when compared to the disclosed sequence, but that encode an antibody that is essentially bioequivalent to the antibodies disclosed herein.

The present disclosure includes formulations comprising antibodies that are bioequivalent to any of the exemplary antibodies set forth herein. Two antibodies are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antibodies are bioequivalent if there are no clinically meaningful differences in their safety, purity, and potency.

In one embodiment, two antibodies are bioequivalent if a patient can be switched one or more times between the first antibody (e.g., reference product) and the second antibody (e.g., biological product) without an expected increase in the risk of adverse effects, including a clinically significant change in immunogenicity, or diminished effectiveness, as compared to continued therapy without such switching.

In one embodiment, two antibodies are bioequivalent if they both act by a common mechanism or mechanisms of action for the condition or conditions of use, to the extent that such mechanisms are known.

Bioequivalence may be demonstrated by in vivo and in vitro methods. Non-limiting examples of bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

### Formulation Excipients and pH

The pharmaceutical formulations of the present disclosure comprise one or more excipients. The term “excipient,” as used herein, means any non-therapeutic agent added to the formulation to provide a desired consistency, viscosity or stabilizing effect.

The pharmaceutical formulations of the present disclosure may also comprise a buffer or buffer system, which serves to maintain a stable pH and to help stabilize the anti-Fel d1 antibody or antibodies. In some embodiments, the buffer or buffer system comprises at least one buffer that has a buffering range that overlaps fully or in part the range of pH 5.5 to 6.3 (e.g., a histidine buffer or a phosphate buffer). In various embodiments, the pH of the formulation is 5.5, 5.6, 5.7, 5.8, 5.9, 6.0, 6.1, 6.2 or 6.3. In some embodiment, the formulations have a pH of 5.8±0.3, e.g., a pH of 5.8±0.2 or a pH of 5.8±0.1. In some embodiment, the formulations have a pH of 5.9±0.3, e.g., a pH of 5.9±0.2 or a pH of 5.9±0.1. In some embodiment, the formulations have a pH of 6.0±0.3, e.g., a pH of 6.0±0.2 or a pH of 6.0±0.1.

In certain embodiments, the buffer comprises histidine. In the context of this disclosure, “histidine buffer” or “buffer comprising histidine” is a buffer comprising the amino acid histidine. Examples of histidine buffers include histidine chloride, histidine acetate, histidine phosphate, and histidine sulfate. In one embodiment, the histidine buffer is prepared by dissolving L-histidine and L-histidine hydrochloride (e.g., as monohydrate) in a defined amount and ratio. In one embodiment, the histidine buffer is prepared by titrating L-histidine (free base, solid) with diluted hydrochloric acid. The term “histidine” is used interchangeably with “histidine buffer” throughout this disclosure. In certain embodiments, the buffer (e.g., histidine) is present at a concentration of from about 1 mM to about 100 mM, e.g., about 1 mM to about 70 mM, about 1 mM to about 40 mM, about 1 mM to about 30 mM, about 5 mM to about 25 mM; or about 5 mM to about 20 mM. In some embodiments, the buffer includes a histidine buffer at a concentration of from 5 mM to 15 mM. In some embodiments, the buffer includes a histidine buffer at a concentration of 10 mM±2 mM.

The pharmaceutical formulations of the present disclosure may comprise one or more thermal stabilizers. In some embodiments, the term “stabilizes” means maintaining greater than about 91% of the antibody in a native conformation when the solution containing the antibody and the thermal stabilizer is incubated at an elevated temperature of about 37° C., about 40° C., or about 45° C. for at least about 28 days (e.g., for one month). In some embodiments, what is meant by “stabilizes” is wherein less than about 6% (e.g., less than 5%, less than 4%, less than 3%, or less than 2.5% of the antibody is aggregated when the solution containing the antibody and the thermal stabilizer is incubated at an elevated temperature of about 37° C., about 40° C., or about 45° C. for at least about 28 days (e.g., for one month). As used herein, “native” means the major or main form of the antibody by size exclusion, which is generally an intact monomer of the antibody. The term “native” also refers to non-aggregated and non-degraded form of the antibody.

In some embodiments, the thermal stabilizer is a polyol such as sorbitol. In some embodiments, the thermal stabilizer is an amino acid such as proline. In some aspects, the thermal stabilizer is a sugar such as sucrose or trehalose. The amount of stabilizer contained within the formulation can vary depending on the specific circumstances and intended purposes for which the formulation is used. In certain embodiments, the thermal stabilizer may be present in the formulation in an amount from about 0.1% to about 20%; about 0.5% to about 20%; about 1% to about 20%; about 1% to about 10%; about 2% to about 15%; about 3% to about 8%; or about 4% to about 6%. For example, the pharmaceutical formulations of the present disclosure may comprise about 0.5%; about 1.0%; about 1.5%; about 2.0%; about 2.5%; about 3.0%; about 3.5%; about 4.0%; about 4.5%; about 5.0%; about 5.5%; about 6.0%; about 6.5%; about 7.0%; about 7.5%; about 8.0%; about 8.5%; about 9.0%; about 9.5%; about 10.0%; about 15%; or about 20% thermal stabilizer (e.g., sucrose). In some embodiments, the formulations contain up to about 10% thermal stabilizer (e.g., sucrose). In some embodiments, the formulations contain from about 1% to about 10% thermal stabilizer (e.g., sucrose), e.g., about 3%, about 5%, about 7.5%, or about 10%. In some embodiments, the formulations contain about 5% thermal stabilizer (e.g., sucrose). In some embodiments, the formulations contain about 5%±1% thermal stabilizer (e.g., sucrose). In some embodiments, the formulations contain about 5%±0.5% thermal stabilizer (e.g., sucrose). Each of the percentages noted above corresponds to a percent weight/volume (w/v).

The pharmaceutical formulations of the present disclosure may comprise one or more viscosity reducers. In some embodiments, the viscosity modifier is an amino acid or a salt. Exemplary amino acids that can be used as viscosity modifiers include, but are not limited to, alanine, proline, arginine, histidine, glycine, and lysine. Exemplary salts (e.g., inorganic salts) that can be used as viscosity modifiers include, but are not limited to, sodium chloride, calcium chloride, magnesium chloride, and calcium acetate. In some embodiments, the viscosity modifier is an amino acid in its salt form (e.g., arginine hydrochloride, histidine hydrochloride, or histidine acetate).

In certain embodiments, the viscosity modifier is present in an amount of at least 50 mM, e.g., at least 60 mM, at least 65 mM, at least 70 mM, or at least 75 mM. In some embodiments, the viscosity modifier is present in an amount from about 50 mM to about 200 mM, e.g., from about 50 mM to about 175 mM, from about 60 mM to about 150 mM, or from about 60 mM to about 130 mM. In some embodiments, the viscosity modifier (e.g., arginine or arginine hydrochloride, or sodium hydrochloride) is present in an amount of at least 60 mM, e.g., at least 65 mM, at least 70 mM, or from about 70 mM to about 120 mM. In certain embodiments, the viscosity modifier is present in an amount from about 0.5% w/v to about 5% w/v, e.g., from about 1% w/v to about 5% w/v. In one embodiment, the viscosity modifier (e.g., proline) is present in an amount from about 1% w/v to about 5% w/v (e.g., about 1.5% or about 3%).

The pharmaceutical formulations of the present disclosure may also comprise one or more surfactants in a type and in an amount that stabilizes the anti-Fel d1 antibody or antibodies under conditions of rough handling or agitation. As used herein, the term “surfactant” refers to a substance that reduces absorption of the hydrophobic antibody patches to other hydrophobic surfaces, and/or reduces the aggregation propensity at the air-water interfaces where hydrophobic antibody regions would tend to undergo unfolding events. Surfactants can be ionic or non-ionic. Specific non-ionic surfactants that can be included in the formulations of the present disclosure include, e.g., polysorbates such as polysorbate 20, polysorbate 28, polysorbate 40, polysorbate 60, polysorbate 65, polysorbate 80, polysorbate 81, and polysorbate 85; poloxamers such as poloxamer 181, poloxamer 188, poloxamer 407; or polyethylene glycols (PEGs) such as PEG3350. Polysorbate 20 is also known as TWEEN 20, sorbitan monolaurate, or polyoxyethylenesorbitan monolaurate. Poloxamer 188 is also known as KOLLIPHOR® P 188 (BASF) or PLURONIC™ F-68 (Gibco).

The amount of surfactant contained within the pharmaceutical formulations of the present disclosure may vary depending on the specific properties desired of the formulations, as well as the particular circumstances and purposes for which the formulations are intended to be used. In certain embodiments, the formulations may contain 0.01% to 0.5% surfactant, e.g., about 0.05% to about 0.5%, from about 0.05% to about 0.25%, from about 0.075% to about 0.25%, or from about 0.075% to about 0.2%. For example, the formulations of the present disclosure may comprise about 0.01%; about 0.02%; about 0.03%; about 0.04%; about 0.05%; about 0.06%; about 0.07%; about 0.08%; about 0.09%; about 0.1%; about 0.11%; about 0.12%; about 0.13%; about 0.14%; about 0.15%; about 0.16%; about 0.17%; about 0.18%; about 0.19%; about 0.20%; about 0.21%; about 0.22%; about 0.23%; about 0.24%; about 0.25%; about 0.26%; about 0.27%; about 0.28%; about 0.29%; about 0.30%; about 0.35%; about 0.40%; about 0.45%; about 0.46%; about 0.47%; about 0.48%; about 0.49%; or about 0.50% of the surfactant. In some embodiments, the surfactant is a polysorbate, e.g., polysorbate 20 or polysorbate 80. In some embodiments, the formulations contain about 0.1% to about 0.2% polysorbate 20 or polysorbate 80. In some embodiments, the formulations contain about 0.1% or about 0.15% polysorbate 20 or polysorbate 80. Each of the percentages noted above corresponds to a percent weight/volume (w/v).

### Exemplary Formulations

In some embodiments, a stable liquid pharmaceutical formulation comprises at least one antibody that specifically binds to Fel d1 and a buffer that maintains a stable pH of about 6.0 (e.g., pH 6.0±0.3).

In some embodiments, a stable liquid pharmaceutical formulation comprises 5-250 mg/mL (e.g., 20 mg/mL, 50 mg/mL, 75 mg/mL, 100 mg/mL, 150 mg/mL, or 200 mg/mL) of at least one antibody that specifically binds to Fel d1 and about 5 mM to about 20 mM of a buffer that maintains a stable pH of about 6.0 (e.g., pH 6.0±0.3). In some embodiments, the buffer is histidine.

In some embodiments, a stable liquid pharmaceutical formulation comprises 5-250 mg/mL (e.g., 20 mg/mL, 50 mg/mL, 75 mg/mL, 100 mg/mL, 150 mg/mL, or 200 mg/mL) of at least one antibody that specifically binds to Fel d1; about 5 mM to about 20 mM of a buffer that maintains a stable pH of about 6.0 (e.g., pH 6.0±0.3); and about 1% to about 10% of a cryoprotectant. In some embodiments, the buffer is histidine. In some embodiments, the cryoprotectant is sucrose, proline, trehalose, or sorbitol.

In some embodiments, a stable liquid pharmaceutical formulation comprises 5-250 mg/mL (e.g., 20 mg/mL, 50 mg/mL, 75 mg/mL, 100 mg/mL, 150 mg/mL, or 200 mg/mL) of at least one antibody that specifically binds to Fel d1; about 5 mM to about 20 mM of a buffer that maintains a stable pH of about 6.0 (e.g., pH 6.0±0.3); and about 0.01% to about 0.5% of a surfactant. In some embodiments, the buffer is histidine. In some embodiments, the surfactant is a polysorbate, e.g., polysorbate 20 or polysorbate 80.

In some embodiments, a stable liquid pharmaceutical formulation comprises 5-250 mg/mL (e.g., 20 mg/mL, 50 mg/mL, 75 mg/mL, 100 mg/mL, 150 mg/mL, or 200 mg/mL) of at least one antibody that specifically binds to Fel d1; about 5 mM to about 20 mM of a buffer that maintains a stable pH of about 6.0 (e.g., pH 6.0±0.3); and about 50 mM to about 200 mM of a viscosity reducer. In some embodiments, the buffer is histidine. In some embodiments, the viscosity reducer is arginine, histidine, or sodium chloride.

In some embodiments, a stable liquid pharmaceutical formulation comprises 5-250 mg/mL (e.g., 20 mg/mL, 50 mg/mL, 75 mg/mL, 100 mg/mL, 150 mg/mL, or 200 mg/mL) of at least one antibody that specifically binds to Fel d1; about 5 mM to about 20 mM of a buffer that maintains a stable pH of about 6.0 (e.g., pH 6.0±0.3); about 1% to about 10% of a cryoprotectant; and about 50 mM to about 200 mM of a viscosity reducer. In some embodiments, the buffer is histidine. In some embodiments, the cryoprotectant is sucrose, proline, trehalose, or sorbitol. In some embodiments, the viscosity reducer is arginine, histidine, or sodium chloride.

In some embodiments, a stable liquid pharmaceutical formulation comprises 5-250 mg/mL (e.g., 20 mg/mL, 50 mg/mL, 75 mg/mL, 100 mg/mL, 150 mg/mL, or 200 mg/mL) of at least one antibody that specifically binds to Fel d1; about 5 mM to about 20 mM of a buffer that maintains a stable pH of about 6.0 (e.g., pH 6.0±0.3); about 0.01% to about 0.5% of a surfactant; and about 50 mM to about 200 mM of a viscosity reducer. In some embodiments, the buffer is histidine. In some embodiments, the surfactant is a polysorbate, e.g., polysorbate 20 or polysorbate 80. In some embodiments, the viscosity reducer is arginine, histidine, or sodium chloride.

In some embodiments, a stable liquid pharmaceutical formulation comprises 5-250 mg/mL (e.g., 20 mg/mL, 50 mg/mL, 75 mg/mL, 100 mg/mL, 150 mg/mL, or 200 mg/mL) of at least one antibody that specifically binds to Fel d1; about 5 mM to about 20 mM of a buffer that maintains a stable pH of about 6.0 (e.g., pH 6.0±0.3); about 1% to about 10% of a cryoprotectant; about 0.01% to about 0.5% of a surfactant; and about 50 mM to about 200 mM of a viscosity reducer. In some embodiments, the buffer is histidine. In some embodiments, the cryoprotectant is sucrose, proline, trehalose, or sorbitol. In some embodiments, the surfactant is a polysorbate, e.g., polysorbate 20 or polysorbate 80. In some embodiments, the viscosity reducer is arginine, histidine, or sodium chloride.

In some embodiments, a stable liquid pharmaceutical formulation comprises 5 mg/mL, 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, or 200 mg/mL of at least one antibody that specifically binds to Fel d1 and a 10 mM histidine buffer that maintains a stable pH of about 6.0 (e.g., pH 6.0±0.3, or a pH of 5.8 or 5.9). In some embodiments, the formulation further comprises 5% (w/v) sucrose. In some embodiments, the formulation further comprises 0.1% (w/v) polysorbate 80.

In some embodiments, a stable liquid pharmaceutical formulation comprises REGN1908 at a concentration of about 150 mg/mL±15 mg/mL and a buffer that maintains a stable pH of about 6.0 (e.g., pH 6.0±0.3, or a pH 5.8 or pH 6.2). In some embodiments, the buffer is 10 mM histidine.

In some embodiments, a stable liquid pharmaceutical formulation comprises REGN1909 at a concentration of about 150 mg/mL±15 mg/mL and a buffer that maintains a stable pH of about 6.0 (e.g., pH 6.0±0.6, or a pH 5.4, 5.6, 5.8, 6.0, 6.2, or 6.5). In some embodiments, the buffer is 10 mM histidine.

In some embodiments, a stable liquid pharmaceutical formulation comprises REGN1908 and/or REGN1909 and a viscosity reducer. In some embodiments, the viscosity reducer is arginine-HCl, proline, or sodium chloride. In some embodiments, the formulation comprises is 70 mM arginine-HCl. In some embodiments, the formulation comprises 3% proline. In some embodiments, the formulation comprises 70 mM sodium chloride.

In some embodiments, a stable liquid pharmaceutical formulation comprises 150 mg/mL±15 mg/mL REGN1908 or REGN1909 and a buffer that maintains a stable pH of about 6.0 (e.g., pH 6.0±0.3, or a pH 5.8 or pH 6.2). In some embodiments, the buffer is 10 mM histidine. In some embodiments, the formulation further comprises one of the following: (i) 10 mM histidine (i.e., no viscosity reducer); (ii) 10 mM histidine+70 mM arginine-HCl; (iii) 100 mM histidine; (iv) 10 mM histidine+3% proline; or (v) 10 mM histidine+70 mM sodium chloride. In some embodiments, the formulation comprises 150 mg/mL REGN1908 and 70 mM arginine, 100 mM histidine or 70 mM sodium chloride. In some embodiments, the formulation comprises 150 mg/mL REGN1908 and 70 mM arginine. In some embodiments, the formulation comprises 150 mg/mL REGN1909 and a viscosity reducer effective in maintaining a viscosity of less than 10 cP in the formulation.

In some embodiments, a stable liquid pharmaceutical formulation comprises 150 mg/mL REGN1908 or REGN1909 and a buffer that maintains a stable pH of about 6.0 (e.g., pH 6.0±0.3, or a pH 5.8 or pH 6.2). In some embodiments, the buffer is 10 mM histidine. In some embodiments the formulation further comprises a cryoprotectant. In some aspects, the cryoprotectant is sucrose at 5% w/v or 10% w/v. In some aspects, the formulation comprises 70 mM arginine-HCl in the presence of sucrose, for example, 5% w/v sucrose.

In some embodiments, a stable liquid pharmaceutical formulation comprises 175 mg/mL REGN1908 or REGN1909 and a buffer that maintains a stable pH of about 6.0 (e.g., pH 6.0±0.3, or a pH 5.8 or pH 6.2). In some embodiments, the formulation further comprises 10 mM histidine. In some embodiments, the formulation further comprises 70 mM arginine. In some embodiments, the formulation further comprises 5% sucrose.

In some embodiments, anti-Fel d1 antibodies (e.g., REGN1908 and REGN1909) are individually formulated in an aqueous buffered formulation comprising: (a) from 5 mM to 20 mM histidine buffer, (b) from 60 mM to 130 mM arginine, and (c) from 50 to 200 mg anti-Fel d1 antibody, at pH 6.0±0.3. In some embodiments, the formulation further comprises from 2.5% to 7.5% w/v sucrose and/or from 0.01% to 0.5% w/v polysorbate.

In some embodiments, anti-Fel d1 antibodies (e.g., REGN1908 and REGN1909) are individually formulated in an aqueous buffered formulation comprising: (a) from 5 mM to 20 mM histidine buffer, (b) from 2.5% to 7.5% w/v sucrose, (c) from 60 mM to 130 mM arginine, and (d) from 50 to 200 mg anti-Fel d1 antibody, at pH 6.0±0.3. In some embodiments, the formulation further comprises from 0.01% to 0.5% w/v polysorbate.

In some embodiments, an anti-Fel d1 antibody formulation comprises: (a) 10 mM±2 mM histidine, (b) 5%±1% w/v sucrose, (c) 70 mM±14 mM arginine, and (d) 190 mg/mL±10 mg/mL REGN1908, at pH 6.0±0.3.

In some embodiments, an anti-Fel d1 antibody formulation comprises: (a) 10 mM±2 mM histidine, (b) 5%±1% w/v sucrose, (c) 70 mM±14 mM arginine, (d) 0.15%±0.075% w/v polysorbate 80, and (e) 150 mg/mL±15 mg/mL REGN1908, at pH 6.0±0.3.

In some embodiments, an anti-Fel d1 antibody formulation comprises: (a) 10 mM±2 mM histidine, (b) 5%±1% w/v sucrose, (c) 70 mM±14 mM arginine, and (d) 190 mg/mL±10 mg/mL REGN1909, at pH 6.0±0.3.

In some embodiments, an anti-Fel d1 antibody formulation comprises: (a) 10 mM±2 mM histidine, (b) 5%±1% w/v sucrose, (c) 70 mM±14 mM arginine, (d) 0.15%±0.075% w/v polysorbate 80, and (e) 150 mg/mL±15 mg/mL REGN1909, at pH 6.0±0.3.

In some aspects, a stable liquid pharmaceutical formulation comprises two anti-Fel d1 antibodies at a target total antibody concentration of 150 mg/mL (75 mg/mL REGN1908 and 75 mg/mL REGN1909) and a buffer that maintains a stable pH of about 6.0 (e.g., pH 6.0±0.3). In some embodiments, the buffer is 10 mM histidine. In some embodiments, the formulation has a pH of 6.1. In some embodiments, the formulation comprises 10 mM histidine and 5% sucrose. In some embodiments, the formulation further comprises either no arginine or 20 mM, 50 mM, 70 mM, 100 mM, or 130 mM arginine.

In some embodiments, a stable liquid pharmaceutical formulation comprises two anti-Fel d1 antibodies at a target total antibody concentration of 150 mg/mL (75 mg/mL REGN1908 and 75 mg/mL REGN1909) and a buffer that maintains a stable pH of about 6.0 (e.g., pH 6.0±0.3). In some embodiments, the formulation further comprises 10 mM histidine, and 70 mM arginine hydrochloride. In some embodiments. In some embodiments, the formulation further comprises either no polysorbate, or polysorbate 20 or polysorbate 80 in an amount of 0.01%, 0.025%, 0.05%, 0.075%, 0.1%, or 0.2% (all percentages w/v). In some embodiments, the formulation comprises 0.05% w/v polysorbate 20, and/or the formulation comprises 0.025% w/v or 0.075% w/v polysorbate 80.

In some embodiments a stable liquid pharmaceutical formulation comprises 150 mg/mL REGN1908-REGN1909 (75 mg/mL each antibody), 10 mM histidine, 70 mM arginine hydrochloride, and 0.15% (w/v) polysorbate at pH 6.0. In some embodiments, no cryoprotectant is added. In some embodiments, 5% sucrose, 3% proline, 5% trehalose, or 2.5% sorbitol is added (all percentages w/v).

In some embodiments, a stable liquid pharmaceutical formulation comprises two anti-Fel d1 antibodies in an aqueous buffered formulation comprising: (a) from 5 mM to 20 mM histidine buffer, (b) from 2.5% to 7.5% w/v sucrose, (c) from 60 mM to 130 mM arginine, (d) from 0.01% to 0.5% w/v polysorbate, and (e) from 50 to 200 mg total anti-Fel d1 antibody, at pH 6.0±0.3.

In some embodiments, a stable liquid formulation comprises: (a) 10 mM±2 mM histidine, (b) 5%±1% w/v sucrose, (c) 70 mM±14 mM arginine, (d) 0.15%±0.075% w/v polysorbate 80, and (e) 150 mg/mL±15 mg/mL REGN1908-REGN1909 (75 mg/mL REGN1908+75 mg/mL REGN1909), at pH 6.0±0.3.

### Characterization of the Pharmaceutical Formulations

In some embodiments, an anti-Fel D1 antibody formulation exhibits one or more of the following attributes:
A formulation that is stable after being subjected to stress, for example temperature cycles, extreme temperatures, agitation during transport, etc.
A formulation having a concentration of anti-Fel D1 antibody/antibodies that is sufficiently high to support delivery of a single dose of the antibody/antibodies to the patient (e.g., a dose of 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1000 mg).
A formulation having a viscosity that is sufficiently low to be administered by intravenous or subcutaneous injection, even at a high antibody concentration (e.g., an antibody concentration of 100-200 mg/mL, or 150-175 mg/mL).
A formulation that is at or near physiological osmolality to support administration by intravenous or subcutaneous injection, even at a high antibody concentration (e.g., an antibody concentration of 100-200 mg/mL, or 150-175 mg/mL).
A formulation that is compatible with and stable in Type 1 clear glass vial and standard serum stopper as packaging.

The pharmaceutical formulations of the present disclosure typically exhibit high levels of stability. The term “stable,” as used herein in reference to the pharmaceutical formulations, means that the antibodies within the pharmaceutical formulations retain an acceptable degree of chemical structure or biological function after storage under defined conditions. A formulation may be stable even though the antibody contained therein does not maintain 100% of its chemical structure or biological function after storage for a defined amount of time. Under certain circumstances, maintenance of about 90%, about 95%, about 96%, about 97%, about 98% or about 99% of an antibody's structure or function after storage for a defined amount of time may be regarded as “stable.”

In some embodiments, “stability” refers to physical stability. Physical stability of a formulation refers to properties such as color, appearance, pH, turbidity, and protein concentration. The presence of visible particulates in solution can be detected by visual inspection, while microscopy, light obscuration (HIAC), or micro-flow imaging (MFI) can be used to measure subvisible particulates. A solution passes visual inspection if it is clear to slightly opalescent, essentially free from visible particulates, and colorless to pale yellow. In addition, turbidity, measured by OD at 405 nm, can also be used to detect particulates in solution. An increase in OD at 405 nm may indicate the presence of particulates, an increase in opalescence, or color change of the test articles. In some embodiments, antibody protein concentration can be measured by a RP-UPLC assay and reported as percent protein recovery relative to the starting material. In the RP-UPLC assay, the antibody is eluted from the RP column as a single peak. The protein concentration is determined from the antibody total peak area by comparing it with a calibration curve generated using antibody standards. Percent of recovery is calculated based on the measured protein concentration relative to the starting protein concentration. In some embodiments, antibody protein concentration is measured by SoloVPE (Repligen Corp., Bridgewater, NJ).

In some embodiments, “stability” refers to chemical stability. Chemical stability refers to the formation of covalently modified forms (e.g., covalent aggregates, cleavage products, or charge variant forms) and non-covalently modified forms (e.g., non-covalent aggregates) of protein. Higher and lower molecular weight degradation products can be separated from native antibody by methods such as size exclusion ultra performance liquid chromatography (SE-UPLC) and microchip electrophoresis (MCE-SDS). The percentage of degraded antibody in the SE-UPLC and MCE-SDS methods is calculated from the ratio of the area of all non-native peaks to the total area of all antibody peaks. Charge variant forms of antibodies are resolved using CEX-UPLC and CEF. In the CEX-UPLC method, peaks with retention times earlier than that of the main peak are labeled as “acidic” peaks; the peaks with retention times later than that of the main peak are labeled as “basic” peaks. In the iCIEF method, peaks that are focused to a pI lower than that of the main peak are labeled “acidic” peaks, whereas those focused to a pI higher than that of the main peak are labeled “basic” peaks.

In some embodiments, stability is measured under stress conditions. For example, the formulation can be subjected to agitation stress (e.g., vortexing at 1000 rpm for 5 or 10 minutes, or using an orbital shaker at 250 rpm for 24 or 48 hours), accelerated stability testing (e.g., subjecting the formulation to elevated temperature, humidity, and/or pH conditions), or thermal stress (e.g., subjecting the formulation to multiple freeze-thaw cycles).

In some embodiments, stability is measured by determining the percentage of native antibody that remains in the formulation after storage for a defined amount of time at a defined temperature. The percentage of native antibody can be determined by, inter alia, size exclusion chromatography (e.g., SE-UPLC), such that native means non-aggregated and non-degraded. An “acceptable degree of stability,” as that phrase is used herein, means that at least 90% of the native form of the antibody can be detected in the formulation after storage for a defined amount of time at a given temperature. In certain embodiments, at least about 90%, 91%, 92%, 93%, 94%, 95%, 96%, 97%, 98%, 99% or 100% of the native form of the antibody can be detected in the formulation after storage for a defined amount of time at a defined temperature. The defined amount of time after which stability is measured can be at least 14 days, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, or more. The defined temperature at which the pharmaceutical formulation may be stored when assessing stability can be any temperature from about −80° C. to about 60° C., e.g., storage at about −80° C., about −30° C., about −20° C., about 0° C., about 4°-8° C., about 5° C., about 25° C., about 35° C., about 37° C., about 40° C., or about 45° C. For example, a pharmaceutical formulation may be deemed stable if after 28 days of storage at 40° C./75% humidity (RH), greater than about 95%, 96%, 97% or 98% of native antibody is detected by SE-UPLC. A pharmaceutical formulation may be deemed stable if after 12 months of storage at 5° C., greater than about 95%, 96%, 97% or 98% of native antibody is detected by SE-UPLC. A pharmaceutical formulation may also be deemed stable if after 3 months of storage at 25° C., greater than about 95%, 96%, 97% or 98% of native antibody is detected by SE-UPLC. A pharmaceutical formulation may also be deemed stable if after 28 days of storage at 45° C., greater than about 89%, 90%, 91%, 92%, 93%, 94%, 95% or 96% of native antibody is detected by SE-UPLC. A pharmaceutical formulation may also be deemed stable if after 12 months of storage at −20° C., greater than about 96%, 97%, or 98% of native antibody is detected by SE-UPLC. A pharmaceutical formulation may also be deemed stable if after 12 months of storage at −30° C., greater than about 96%, 97% or 98% of native antibody is detected by SE-UPLC. A pharmaceutical formulation may also be deemed stable if after 12 months of storage at −80° C., greater than about 96%, 97% or 98% of native antibody is detected by SE-UPLC.

In some embodiments, stability is measured by determining the percentage of antibody that forms in an aggregate within the formulation after storage for a defined amount of time at a defined temperature, wherein stability is inversely proportional to the percent aggregate that is formed. The percentage of aggregated antibody can be determined by, inter alia, size exclusion chromatography (e.g., SE-UPLC). An “acceptable degree of stability”, as that phrase is used herein, means that at most 5% of the antibody is in an aggregated form (also denoted as the high molecular weight (“HMW”) form) detected in the formulation after storage for a defined amount of time at a given temperature. In certain embodiments an acceptable degree of stability means that at most about 5%, 4%, 3%, 2%, 1%, 0.5%, or 0.1% of the antibody can be detected in an aggregate in the formulation after storage for a defined amount of time at a given temperature. The defined amount of time after which stability is measured can be at least 2 weeks, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, or more. The temperature at which the pharmaceutical formulation may be stored when assessing stability can be any temperature from about −80° C. to about 45° C., e.g., storage at about −80° C., about −30° C., about −20° C., about 0° C., about 4°-8° C., about 5° C., about 25° C., about 35° C., about 37° C., about 40° C., or about 45° C. For example, a pharmaceutical formulation may be deemed stable if after 12 months of storage at 5° C., less than about 2%, 1%, 0.5%, or 0.1% of the antibody is detected in an aggregated form.

In some embodiments, stability is measured by determining the percentage of antibody that migrates in a more acidic fraction during ion exchange (“acidic form”) than in the main fraction of antibody (“main charge form”), wherein stability is inversely proportional to the fraction of antibody in the acidic form. While not wishing to be bound by theory, deamidation of the antibody may cause the antibody to become more negatively charged and thus more acidic relative to the non-deamidated antibody (see, e.g., Robinson, PNAS, Apr. 16, 2002, 99:5283-5288). The percentage of “acidified” antibody can be determined by, inter alia, ion exchange chromatography (e.g., cation exchange ultra performance liquid chromatography [CEX-UPLC]). An “acceptable degree of stability,” as that phrase is used herein, means that at most 45% of the antibody is in a more acidic form detected in the formulation after storage for a defined amount of time at a defined temperature. In certain embodiments an acceptable degree of stability means that at most about 40%, 35%, 30%, 25%, 20%, 15%, 10%, 5%, 4%, 3%, 2%, 1%, 0.5%, or 0.1% of the antibody can be detected in an acidic form in the formulation after storage for a defined amount of time at a given temperature. In one embodiment, an acceptable degree of stability means that less than 30%, 25%, 20%, 15%, 10%, 5%, 4%, 3%, 2%, 1%, 0.5%, or 0.1% of the antibody can be detected in an acidic form in the formulation after storage for a defined amount of time at a given temperature. The defined amount of time after which stability is measured can be at least 2 weeks, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, or more. The temperature at which the pharmaceutical formulation may be stored when assessing stability can be any temperature from about −80° C. to about 45° C., e.g., storage at about −80° C., about −30° C., about −20° C., about 0° C., about 4°-8° C., about 5° C., about 25° C., or about 45° C. For example, a pharmaceutical formulation may be deemed stable if after three months of storage at −80° C., −30° C., or −20° C. less than about 30%, 29%, 28%, 27%, 26%, 25%, 24%, 23%, 22%, 21%, 20%, 19%, 18%, 17%, 16%, 15%, 14%, 13%, 12%, 10%, 9%, 8%, 7%, 6%, 5%, 4%, 3%, 2%, 1%, 0.5% or 0.1% of the antibody is in a more acidic form. A pharmaceutical formulation may also be deemed stable if after 12 months of storage at 5° C., less than about 32%, 31%, 30%, 29%, 28%, 27%, 26%, 25%, 24%, 23%, 22%, 21%, 20%, 19%, 18%, 17%, 16%, 15%, 14%, 13%, 12%, 10%, 9%, 8%, 7%, 6%, 5%, 4%, 3%, 2%, 1%, 0.5% or 0.1% of the antibody is in a more acidic form. A pharmaceutical formulation may also be deemed stable if after 3 months of storage at 25° C., less than about 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 10%, 9%, 8%, 7%, 6%, 5%, 4%, 3%, 2%, 1%, 0.5% or 0.1% of the antibody is in a more acidic form. A pharmaceutical formulation may also be deemed stable if after 28 days of storage at 45° C., less than about 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 10%, 9%, 8%, 7%, 6%, 5%, 4%, 3%, 2%, 1%, 0.5% or 0.1% of the antibody can be detected in a more acidic form.

Other methods may be used to assess the stability of the formulations of the present disclosure such as, e.g., differential scanning calorimetry (DSC) to determine thermal stability, controlled agitation to determine mechanical stability, and absorbance at about 350 nm or about 405 nm to determine solution turbidities. For example, a formulation of the present disclosure may be considered stable if, after 6 or more months of storage at about 5° C. to about 25° C., the change in OD₄₀₅ of the formulation is less than about 0.05 (e.g., 0.04, 0.03, 0.02, 0.01, or less) from the OD₄₀₅ of the formulation at time zero.

Measuring the binding affinity of the antibody to its target or the biological activity of the antibody may also be used to assess stability or potency. For example, a formulation of the present disclosure may be regarded as stable if, after storage at e.g., 5° C., 25° C., 45° C., etc. for a defined amount of time (e.g., 1 to 36 months), the anti-Fel d1 antibody or antibodies contained within the formulation binds to Fel d1 antigen with an affinity that is at least 90%, 95%, or more of the binding affinity of the antibody prior to said storage. In some embodiments, the potency of an antibody in a formulation is assessed relative to a reference standard (e.g., the antibody or a formulation containing the antibody prior to storage) by ELISA or by bioassay, with an acceptance criteria of the test article relative to the reference standard of 50-150% potency. Binding affinity may be determined by e.g., ELISA or surface plasmon resonance. Biological activity (i.e., the ability of the antibody to inhibit the activity of Fel D1) may be determined by a standard in vitro or in vivo assays (such as a passive cutaneous anaphylaxis assay, disclosed in WO 2013/166236, or a basophil activation test, disclosed in WO 2018/118713).

Additional methods for assessing the stability of an antibody in formulation are demonstrated in the Examples presented below.

The liquid pharmaceutical formulations of the present disclosure may, in certain embodiments, exhibit low to moderate levels of viscosity. “Viscosity” as used herein may be “kinematic viscosity” or “absolute viscosity.” “Kinematic viscosity” is a measure of the resistive flow of a fluid under the influence of gravity. When two fluids of equal volume are placed in identical capillary viscometers and allowed to flow by gravity, a viscous fluid takes longer than a less viscous fluid to flow through the capillary. For example, if one fluid takes 200 seconds to complete its flow and another fluid takes 400 seconds, the second fluid is twice as viscous as the first on a kinematic viscosity scale. “Absolute viscosity,” sometimes called dynamic or simple viscosity, is the product of kinematic viscosity and fluid density (Absolute Viscosity=Kinematic Viscosity×Density). The dimension of kinematic viscosity is L²/T where L is a length and T is a time. Commonly, kinematic viscosity is expressed in centistokes (cSt). The SI unit of kinematic viscosity is mm²/s, which is 1 cSt. Absolute viscosity is expressed in units of centipoise (cP). The SI unit of absolute viscosity is the milliPascal-second (mPa-s), where 1 cP=1 mPa-s.

As used herein, a low level of viscosity, in reference to a fluid formulation of the present disclosure, will exhibit an absolute viscosity of less than about 20 cPoise (cP). For example, a fluid formulation disclosed herein will be deemed to have “low viscosity,” if, when measured using standard viscosity measurement techniques, the formulation exhibits an absolute viscosity of about 20 cP, about 19 cP, about 18 cP, about 17 cP, about 16 cP, about 15 cP, about 14 cP, about 13 cP, about 12 cP, about 11 cP, about 10 cP, about 9 cP, about 8 cP, or less. As used herein, a moderate level of viscosity, in reference to a fluid formulation of the present disclosure, will exhibit an absolute viscosity of between about 35 cP and about 20 cP. For example, a fluid formulation disclosed herein will be deemed to have “moderate viscosity,” if when measured using standard viscosity measurement techniques, the formulation exhibits an absolute viscosity of about 34 cP, about 33 cP, about 32 cP, about 31 cP, about 30 cP, about 29 cP, about 28 cP, about 27 cP, about 26 cP, about 25 cP, about 24 cP, about 23 cP, about 22 cP, about 21 cP, or about 20 cP. In some embodiments, a formulation provided herein has a viscosity ≤30 cP, e.g., ≤25 cP. In some embodiments, a formulation provided herein has a viscosity ≤20 cP, e.g., ≤15 cP.

### Containers and Methods of Administration

The pharmaceutical formulations of the present disclosure may be contained within any container suitable for storage of medicines and other therapeutic compositions. For example, the pharmaceutical formulations may be contained within a sealed and sterilized plastic or glass container having a defined volume such as a vial, ampule, syringe, cartridge, bottle or IV bag. Different types of vials can be used to contain the formulations of the present disclosure including, e.g., clear and opaque (e.g., amber) glass or plastic vials. Likewise, any type of syringe can be used to contain and/or administer the pharmaceutical formulations of the present disclosure. In some embodiments, the pharmaceutical formulation is contained in a prefilled syringe. In some embodiments, the pharmaceutical formulation is contained in a prefilled staked needle syringe. In some embodiments, the pharmaceutical formulation is contained in a container selected from the group consisting of a glass vial, a syringe, a pre-filled syringe, a pen delivery device, and an autoinjector.

The pharmaceutical formulations of the present disclosure may be contained within “normal tungsten” syringes or “low tungsten” syringes. As will be appreciated by persons of ordinary skill in the art, the process of making glass syringes generally involves the use of a hot tungsten rod which functions to pierce the glass thereby creating a hole from which liquids can be drawn and expelled from the syringe. This process results in the deposition of trace amounts of tungsten on the interior surface of the syringe. Subsequent washing and other processing steps can be used to reduce the amount of tungsten in the syringe. As used herein, the term “normal tungsten” means that the syringe contains greater than 500 parts per billion (ppb) of tungsten. The term “low tungsten” means that the syringe contains less than 500 ppb of tungsten. For example, a low tungsten syringe, according to the present disclosure, can contain less than about 490, 480, 470, 460, 450, 440, 430, 420, 410, 390, 350, 300, 250, 200, 150, 100, 90, 80, 70, 60, 50, 40, 30, 20, 10 or fewer ppb of tungsten.

The rubber plungers used in syringes, and the rubber stoppers used to close the openings of vials, may be coated to prevent contamination of the medicinal contents of the syringe or vial and/or to preserve their stability. Thus, pharmaceutical formulations of the present disclosure, according to certain embodiments, may be contained within a syringe that comprises a coated plunger, or within a vial that is sealed with a coated rubber stopper. For example, the plunger or stopper may be coated with a fluorocarbon film. Examples of coated stoppers and/or plungers suitable for use with vials and syringes containing the pharmaceutical formulations of the present disclosure are mentioned in, e.g., U.S. Pat. Nos. 4,997,423; 5,908,686; 6,286,699; 6,645,635; and 7,226,554, the contents of which are incorporated by reference herein in their entireties. Particular exemplary coated rubber stoppers and plungers that can be used in the context of the present disclosure are commercially available under the tradename “FluroTec®,” available from West Pharmaceutical Services, Inc. (Lionville, PA). According to certain embodiments of the present disclosure, the pharmaceutical formulations may be contained within a low tungsten syringe that comprises a fluorocarbon-coated plunger. In some embodiments, the container is a syringe, such as an Ompi EZ-Fill™ syringe or a BD Neopak™ syringe. In some cases, the syringe is a 1 mL long glass syringe with a 1 mL West piston, a 27G thin wall needle and an FM30 needle shield or a BD260 needle shield. In some cases, the syringe is a 2.25 mL glass syringe (e.g., Nuova Ompi). In various embodiments, the syringe is a 0.5 mL, 0.6 mL, 0.7 mL, 0.8 mL, 0.9 mL, 1.0 mL, 1.1 mL, 1.2 mL, 1.3 mL, 1.4 mL, 1.5 mL, 1.6 mL, 1.7 mL, 1.8 mL, 1.9 mL, 2.0 mL, 2.1 mL, 2.2 mL, 2.3 mL, 2.4 mL, 2.5 mL, 2.6 mL, 2.7 mL, 2.8 mL, 2.9 mL, 3.0 mL, 3.5 mL, 4.0 mL, 4.5 mL, 5.0 mL, 5.5 mL, 6.0 mL, 6.5 mL, 7.0 mL, 7.5 mL, 8.0 mL, 8.5 mL, 9.0 mL, 9.5 mL, or 10 mL syringe (e.g., a glass syringe).

The pharmaceutical formulations can be administered to a patient by parenteral routes such as injection (e.g., subcutaneous, intravenous, intramuscular, intraperitoneal, etc.) or percutaneous, mucosal, nasal, pulmonary and/or oral administration. Numerous reusable pen and/or autoinjector delivery devices can be used to subcutaneously deliver the pharmaceutical formulations of the present disclosure. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany), to name only a few. Examples of disposable pen and/or autoinjector delivery devices having applications in subcutaneous delivery of a pharmaceutical composition of the present disclosure include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P.), and the HUMIRA™ Pen (Abbott Labs, Abbott Park, IL), to name only a few. In some cases, the pharmaceutical formulation is contained in a syringe specifically adapted for use with an autoinjector. Subcutaneous injections may be administered using a 20-30 gauge needle, or a 25-30 gauge needle. In some cases, subcutaneous injections may be administered using a 25-gauge needle. In some cases, subcutaneous injections may be administered using a 27-gauge needle. In some cases, subcutaneous injections may be administered using a 29-gauge needle.

Another type of delivery device can include a safety system. Such devices can be relatively inexpensive, and operate to manually or automatically extend a safety sleeve over a needle once injection is complete. Examples of safety systems can include the ERIS device by West Pharmaceutical, or the UltraSafe device by Becton Dickinson. In addition, the use of a large volume device (“LVD”), or bolus injector, to deliver the pharmaceutical formulations of the present disclosure is also contemplated herein. In some cases, the LVD or bolus injector may be configured to inject a medicament into a patient. For example, an LVD or bolus injector may be configured to deliver a “large” volume of medicament (typically about 2 mL to about 10 mL).

The pharmaceutical formulations of the present disclosure can also be contained in a unit dosage form. The term “unit dosage form,” as used herein, refers to a physically discrete unit suitable as a unitary dosage for the patient to be treated, each unit containing a predetermined quantity of active compound calculated to produce the desired therapeutic effect in association with the required pharmaceutical carrier, diluent, or excipient. In various embodiments, the unit dosage form is contained within a container as discussed herein. Actual dosage levels of the active ingredient (e.g., an anti-Fel d1 antibody) in the formulations of the present disclosure may be varied so as to obtain an amount of the active ingredient which is effective to achieve the desired therapeutic response for a particular patient, composition, and mode of administration, without adverse effect to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. The term “diluent” as used herein refers to a solution suitable for altering or achieving an exemplary or appropriate concentration or concentrations as described herein.

In various embodiments, the unit dosage form contains an amount of the active ingredient or ingredients (e.g., one or more anti-Fel d1 antibodies) intended for a single use. In various embodiments, the amount of the active ingredient(s) in the unit dosage form is from about 0.1 mg to about 5000 mg, from about 25 mg to about 1000 mg, from about 50 mg to about 500 mg, or from about 100 to 750 mg, or ranges or intervals thereof. For example, ranges of values using a combination of any of the above recited values (or values contained within the above recited ranges) as upper and/or lower limits are intended to be included. In a particular embodiment, the formulation often is supplied as a liquid in unit dosage form. In some embodiments, the unit dosage form contains about 25 mg, about 50 mg, about 75 mg, about 100 mg, about 125 mg, about 150 mg, about 175 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg of the active ingredient(s). In some embodiments, the unit dosage form contains two anti-Fel d1 antibodies and contains about 25 mg, about 50 mg, about 75 mg, about 100 mg, about 125 mg, about 150 mg, about 175 mg, about 200 mg, about 250 mg, about 300 mg, about 350 mg, or about 400 mg of each antibody. In some embodiments, a unit dosage form according to the present disclosure is suitable for subcutaneous or intravenous administration to a patient.

The present disclosure also includes methods of preparing a unit dosage form. In an exemplary embodiment, a method for preparing a pharmaceutical unit dosage form includes combining the formulation of any of foregoing embodiments in a suitable container (e.g., those containers discussed herein).

### Kits

Further provided are kits comprising one or more components that include, but are not limited to, a stable liquid pharmaceutical formulation comprising at least one antibody that specifically binds to Fel d1, alone or in association with one or more additional components including, but not limited to, a further therapeutic agent, as discussed herein. The stable liquid pharmaceutical formulation and/or the further therapeutic agent can be formulated as a single composition or separately in two or more compositions, e.g., with a pharmaceutically acceptable carrier, in a pharmaceutical composition.

Illustratively, the kit can comprise a stable liquid pharmaceutical formulation comprising: an antibody that specifically binds to Fel d1, wherein the antibody comprises an HCDR1 comprising the amino acid sequence of SEQ ID NO:4, an HCDR2 comprising the amino acid sequence of SEQ ID NO:6, an HCDR3 comprising the amino acid sequence of SEQ ID NO:8, an LCDR1 comprising the amino acid sequence of SEQ ID NO:12, an LCDR2 comprising the amino acid sequence AAS, and an LCDR3 comprising the amino acid sequence of SEQ ID NO:14.

In some embodiments, the kit can comprise a stable liquid pharmaceutical formulation comprising: an antibody that specifically binds to Fel d1, wherein the antibody comprises an HCDR1 comprising the amino acid sequence of SEQ ID NO:18, an HCDR2 comprising the amino acid sequence of SEQ ID NO:20, an HCDR3 comprising the amino acid sequence of SEQ ID NO:22, an LCDR1 comprising the amino acid sequence of SEQ ID NO:26, an LCDR2 comprising the amino acid sequence KAS, and an LCDR3 comprising the amino acid sequence of SEQ ID NO:28.

In some embodiments, the kit can comprise a stable liquid pharmaceutical formulation comprising:
a first antibody that specifically binds to Fel d1, wherein the first antibody comprises an HCVR comprising the amino acid sequence of SEQ ID NO:2 and an LCVR comprising the amino acid sequence of SEQ ID NO:10, wherein the concentration of the first antibody is up to about 100 mg/mL (e.g., 50 mg/mL±5 mg/mL or 75 mg/mL±10 mg/mL); and
a second antibody that specifically binds to Fel d1, wherein the second antibody comprises an HCVR comprising the amino acid sequence of SEQ ID NO:16 and an LCVR comprising the amino acid sequence of SEQ ID NO:24, wherein the concentration of the second antibody is up to about 100 mg/mL (e.g., 50 mg/mL±5 mg/mL or 75 mg/mL±10 mg/mL).

In one embodiment, the kit includes a stable liquid pharmaceutical formulation in one container (e.g., in a sterile glass or plastic vial). In one embodiment, the kit further includes an additional therapeutic agent in another container (e.g., in a sterile glass or plastic vial).

In another embodiment, the kit comprises a combination of the stable liquid pharmaceutical formulation in combination with one or more further therapeutic agents formulated together, optionally, in a pharmaceutical composition, in a single, common container.

If the kit includes the stable liquid pharmaceutical formulation for parenteral or subcutaneous administration to a subject, the kit can include a device (e.g., an injection device) for performing such administration. For example, the kit can include one or more hypodermic needles or other injection devices as discussed above containing the stable liquid pharmaceutical formulation.

The kit can include a package insert including information concerning the stable liquid pharmaceutical formulation and dosage forms in the kit. Generally, such information aids patients and physicians in using the enclosed pharmaceutical compositions and dosage forms effectively and safely. For example, the following information may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and patent information. In some embodiments, the kit comprises instructions for using the formulation in the treatment of a disease, disorder, or condition associated with Fel d1 expression or activity, e.g., as disclosed herein.

### Therapeutic Uses

The pharmaceutical formulations of the present disclosure are useful, inter alia, for the treatment, prevention, and/or amelioration of any disease, disorder, or condition associated with Fel d1 expression or activity.

The therapeutic methods of the present disclosure comprise administering to a subject any formulation comprising one or more anti-Fel d1 antibodies as disclosed herein. The subject to which the pharmaceutical formulation is administered can be, e.g., any human or non-human animal that is in need of such treatment, prevention and/or amelioration. The present disclosure further includes the use of any of the pharmaceutical formulations disclosed herein in the manufacture of a medicament for the treatment, prevention and/or amelioration of any disease or disorder associated with Fel d1 expression or activity.

In some embodiments, the pharmaceutical formulations of the present disclosure are used for treating, preventing, or ameliorating one or more symptoms associated with exposure to the cat allergen Fel d1. In some embodiments, the pharmaceutical formulations of the present disclosure are used for treating cat-allergic patients, e.g., patients suffering from allergies to cat dander or patients who demonstrate sensitivity to the Fel d1 allergen. In some embodiments, the pharmaceutical formulations of the present disclosure are used to prevent a heightened response to Fel d1 upon secondary exposure, or the accompanying symptoms associated with the allergy, or to lessen the severity and/or the duration of the allergic response associated with a primary exposure to a cat harboring the Fel d1 allergen or with the recurrence of the symptoms upon secondary exposure. In some embodiments, the pharmaceutical formulations of the present disclosure are used for treating or preventing cat allergen-triggered asthma.

In some embodiments, a patient to be treated with a pharmaceutical formulation as disclosed herein is a sensitized to Fel d1 antigen or cat dander. In some embodiments, the patient is a cat-allergic patient. In some embodiments, the patient is a cat-allergic patient with conjunctivitis and rhinitis. In some embodiments, the patient is a cat-allergic patient with rhinitis with or without conjunctivitis symptoms. In some embodiments, the patient is a cat-allergic patient with rhinitis with or without conjunctivitis symptoms and with or without asthma. In some embodiments, the patient is a cat-allergic patient with asthma (e.g., mild asthma). In some embodiments, the patient is ≥12 years of age. In some embodiments, the patient is ≥18 years of age.

In some embodiments, treatment with a pharmaceutical formulation as disclosed herein results in a lessening in severity and/or duration of at least one symptom or complication associated with the allergic reaction against the Fel d1 allergen. In some embodiments, the symptom or complication associated with the allergic reaction is sneezing, rhinorrhea, nasal itching, or nasal congestion. In some embodiments, treatment results in a reduction in allergic rhinitis, allergic conjunctivitis, rhinoconjunctivitis, allergic asthma, asthma exacerbations, or an anaphylactic response following exposure of the patient to an allergen.

In some embodiments, treatment with a pharmaceutical formulation as disclosed herein results in one or more of the following:
(a) reduction in daily total nasal symptom score (TNSS);
(b) reduction in daily ocular symptom score (TOSS);
(c) reduction in daily total symptom score (TSS), calculated as the sum of TNSS and TOSS;
(d) reduction in daily medication score (DMS);
(e) reduction in daily combined symptom and medication score (CSMS), calculated as the daily TSS plus DMS; and/or
(f) reduction in mean wheal diameter of a cat skin prick test (SPT).

In some embodiments, the pharmaceutical formulations of the present disclosure are used in combination with a second therapeutic agent. For example, in some embodiments, the pharmaceutical formulation is used in combination with one or more other therapeutic moieties or modalities known in the art for treating, preventing, or reducing the severity of allergies, such as, but not limited to, treatment with antihistamines, corticosteroids, epinephrine, anti-IgE antibodies (e.g., omalizumab), or allergen-specific immunotherapy (e.g., SCIT or SLIT).

A summary of the sequences and the corresponding SEQ ID NOs referenced herein is shown in Table 1, below.

TABLE 1
Informal Sequence Listing

| SEQ | | |
|---|---|---|
| ID | | |
| NO | Sequence | Description |
| 1 | gaggtgcagc tggtggagtc tgggggaggc ctggtcaagc ctggggggtc cctgagactc | REGN1908 HCVR |
| | tcctgtgcag cctctggatt caccttcaga aactataaca taaactgggt ccgccaggct | nucleic acid sequence |
| | ccagggaagg ggctggagtg ggtctcactc atcagtggta gtagtagtta catatattac | |
| | gcagactcag tgaagggccg attcaccgtc tccagagaca acgccaagaa ctcactgtat | |
| | ctgcaaatga acagcctgag agccgaggac acggctgtgt attactgtgc gaggcggaca | |
| | ttaagctact acgttatgga cgtctggggc caagggacca cggtcaccgt ctcctca | |
| 2 | EVQLVESGGGLVKPGGSLRLSCAASGFTFRNYNINWVRQAPGKGLEWVSLISGSSSYIYYADSVK | REGN1908 HCVR |
| | GRFTVSRDNAKNSLYLQMNSLRAEDTAVYYCARRTLSYYVMDVWGQGTTVTVSS | amino acid sequence |
| 3 | ggattcacct tcagaaacta taac | REGN1908 HCDR1 |
| | | nucleic acid sequence |
| 4 | GFTFRNYN | REGN1908 HCDR1 |
| | | amino acid sequence |
| 5 | atcagtggta gtagtagtta cata | REGN1908 HCDR2 |
| | | nucleic acid sequence |
| 6 | ISGSSSYI | REGN1908 HCDR2 |
| | | amino acid sequence |
| 7 | gcgaggcgga cattaagcta ctacgttatg gacgtc | REGN1908 HCDR2 |
| | | nucleic acid sequence |
| 8 | ARRTLSYYVMDV | REGN1908 HCDR3 |
| | | amino acid sequence |
| 9 | gacatccagg tgacccagtc tccatccccc ctgtctgcat ctgtaggaga cagagtcacc | REGN1908 LCVR |
| | atcacttgcc gggcgagtca gggcattagc aattatttag cctggtatca gcagaaacca | nucleic acid sequence |
| | gggagagttc ctcagctcct gatctatgct gcatccactt tgcaatcagg ggtcccatct | |
| | cggttcagtg gcagtggatc tgggacagat ttcactctca ccatcagcag cctgcagcct | |
| | gaagatgttg caacttatta ctgtcaaaag tataacagtg ccccgtacac ttttggccag | |
| | gggaccaagc tggagatcaa a | |
| 10 | DIQVTQSPSPLSASVGDRVTITCRASQGISNYLAWYQQKPGRVPQLLIYAASTLQSGVPSRFSGS | REGN1908 LCVR |
| | GSGTDFTLTISSLQPEDVATYYCQKYNSAPYTFGQGTKLEIK | amino acid sequence |
| 11 | cagggcatta gcaattat | REGN1908 LCDR1 |
| | | nucleic acid sequence |
| 12 | QGISNY | REGN1908 LCDR1 |
| | | amino acid sequence |
| | gctgcatcc | REGN1908 LCDR2 |
| | | nucleic acid sequence |
| | AAS | REGN1908 LCDR2 |
| | | amino acid sequence |
| 13 | caaaagtata acagtgcccc gtacact | REGN1908 LCDR3 |
| | | nucleic acid sequence |
| 14 | QKYNSAPYT | REGN1908 LCDR3 |
| | | amino acid sequence |
| 15 | gaggtgcagc tggtggagtc tgggggaggc ttggtacagc ctggggggtc cctgagactc | REGN1909 HCVR |
| | tcctgtgcag cctctggatt cacctttagc agttatgcca tgagctgggt ccgccaggct | nucleic acid sequence |
| | ccagggaagg ggctggagtg ggtctcagct attagtggtc gtggttataa cgcagactac | |
| | gcagactccg tgaagggccg gttcaccatc tccagggaca attccaagaa cacgctgtat | |
| | ctgcaaatga acagcctgag agccgaagac acggccgtat attactgtgc gaaattggaa | |
| | tactttgact actggggcca gggaaccctg gtcactgtct cctca | |
| 16 | EVQLVESGGGLVQPGGSLRLSCAASGFTFSSYAMSWVRQAPGKGLEWVSAISGRGYNADYADSVK | REGN1909 HCVR |
| | GRFTISRDNSKNTLYLQMNSLRAEDTAVYYCAKLEYFDYWGQGTLVTVSS | amino acid sequence |
| 17 | ggattcacct ttagcagtta tgcc | REGN1909 HCDR1 |
| | | nucleic acid sequence |
| 18 | GFTFSSYA | REGN1909 HCDR1 |
| | | amino acid sequence |
| 19 | attagtggtc gtggttataa cgca | REGN1909 HCDR2 |
| | | nucleic acid sequence |
| 20 | ISGRGYNA | REGN1909 HCDR2 |
| | | amino acid sequence |
| 21 | gcgaaattgg aatactttga ctac | REGN1909 HCDR2 |
| | | nucleic acid sequence |
| 22 | AKLEYFDY | REGN1909 HCDR3 |
| | | amino acid sequence |
| 23 | gacatccaga tgacccagtc tccttccacc ctgtctgcat ctgtaggaga cagagtcacc | REGN1909 LCVR |
| | atcacttgcc gggccagtca gagtattagt agctggttgg cctggtatca gcagaaacca | nucleic acid sequence |
| | gggaaagccc ctaagctcct gatctataag gcgtctagtt tagaaagtgg ggtcccatca | |
| | aggttcagcg gcagtggatc tgggacagat ttcactctca ccatcagcag cctgaggcct | |
| | gaagattttg caacttatta ctgccaacag tataatagtt accctctgac tttcggcgga | |
| | gggaccaagg tggagatcaa a | |
| 24 | DIQMTQSPSTLSASVGDRVTITCRASQSISSWLAWYQQKPGKAPKLLIYKASSLESGVPSRFSGS | REGN1909 LCVR |
| | GSGTDFTLTISSLRPEDFATYYCQQYNSYPLTFGGGTKVEIK | amino acid sequence |
| 25 | cagagtatta gtagctgg | REGN1909 LCDR1 |
| | | nucleic acid sequence |
| 26 | QSISSW | REGN1909 LCDR1 |
| | | amino acid sequence |
| | aaggcgtct | REGN1909 LCDR2 |
| | | nucleic acid sequence |
| | KAS | REGN1909 LCDR2 |
| | | amino acid sequence |
| 27 | caacagtata atagttaccc tctgact | REGN1909 LCDR3 |
| | | nucleic acid sequence |
| 28 | QQYNSYPLT | REGN1909 LCDR3 |
| | | amino acid sequence |
| 29 | EVQLVESGGGLVKPGGSLRLSCAASGFTFRNYNINWVRQAPGKGLEWVSLISGSSSYIYYADSVK | REGN1908 heavy |
| | GRFTVSRDNAKNSLYLQMNSLRAEDTAVYYCARRTLSYYVMDVWGQGTTVTVSSASTKGPSVFPL | chain amino acid |
| | APCSRSTSESTAALGCLVKDYFPEPVTVSWNSGALTSGVHTFPAVLQSSGLYSLSSVVTVPSSSL | sequence |
| | GTKTYTCNVDHKPSNTKVDKRVESKYGPPCPPCPAPEFLGGPSVFLFPPKPKDTLMISRTPEVTC | |
| | VVVDVSQEDPEVQFNWYVDGVEVHNAKTKPREEQFNSTYRVVSVLTVLHQDWLNGKEYKCKVSNK | |
| | GLPSSIEKTISKAKGQPREPQVYTLPPSQEEMTKNQVSLTCLVKGFYPSDIAVEWESNGQPENNY | |
| | KTTPPVLDSDGSFFLYSRLTVDKSRWQEGNVFSCSVMHEALHNHYTQKSLSLSLGK | |
| 30 | DIQVTQSPSPLSASVGDRVTITCRASQGISNYLAWYQQKPGRVPQLLIYAASTLQSGVPSRFSGS | REGN1908 light chain |
| | GSGTDFTLTISSLQPEDVATYYCQKYNSAPYTFGQGTKLEIKRTVAAPSVFIFPPSDEQLKSGTA | amino acid sequence |
| | SVVCLLNNFYPREAKVQWKVDNALQSGNSQESVTEQDSKDSTYSLSSTLTLSKADYEKHKVYACE | |
| | VTHQGLSSPVTKSFNRGEC | |
| 31 | EVQLVESGGGLVQPGGSLRLSCAASGFTFSSYAMSWVRQAPGKGLEWVSAISGRGYNADYADSVK | REGN1909 heavy |
| | GRFTISRDNSKNTLYLQMNSLRAEDTAVYYCAKLEYFDYWGQGTLVTVSSASTKGPSVFPLAPCS | chain amino acid |
| | RSTSESTAALGCLVKDYFPEPVTVSWNSGALTSGVHTFPAVLQSSGLYSLSSVVTVPSSSLGTKT | sequence |
| | YTCNVDHKPSNTKVDKRVESKYGPPCPPCPAPEFLGGPSVFLFPPKPKDTLMISRTPEVTCVVVD | |
| | VSQEDPEVQFNWYVDGVEVHNAKTKPREEQFNSTYRVVSVLTVLHQDWLNGKEYKCKVSNKGLPS | |
| | SIEKTISKAKGQPREPQVYTLPPSQEEMTKNQVSLTCLVKGFYPSDIAVEWESNGQPENNYKTTP | |
| | PVLDSDGSFFLYSRLTVDKSRWQEGNVFSCSVMHEALHNHYTQKSLSLSLGK | |
| 32 | DIQMTQSPSTLSASVGDRVTITCRASQSISSWLAWYQQKPGKAPKLLIYKASSLESGVPSRFSGS | REGN1909 light chain |
| | GSGTDFTLTISSLRPEDFATYYCQQYNSYPLTFGGGTKVEIKRTVAAPSVFIFPPSDEQLKSGTA | amino acid sequence |
| | SVVCLLNNFYPREAKVQWKVDNALQSGNSQESVTEQDSKDSTYSLSSTLTLSKADYEKHKVYACE | |
| | VTHQGLSSPVTKSFNRGEC | |

### EXAMPLES

The following examples are put forth so as to provide those of ordinary skill in the art with a complete disclosure and description of how to make and use the methods and compositions of the disclosure, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Antibodies to Fel d1

Antibodies specific for Fel d1 were generated as described in U.S. Pat. No. 9,079,948, incorporated by reference herein. Exemplary fully human anti-Fel d1 antibodies (i.e., antibodies possessing human variable domains and human constant domains) that were generated include H4H1232N (REGN1908) and H4H2636P (REGN1909). The nucleic acid and amino acid sequences of the CDRs, heavy chain variable regions, and light chain variable regions of these antibodies are set forth in Table 1. Tables 2-3 below sets forth the amino acid sequence identifiers (Table 2) and nucleic acid sequence identifiers (Table 3) of these antibodies.

TABLE 2
Amino Acid Sequence Identifiers

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| REGN1908 | 2 | 4 | 6 | 8 | 10 | 12 | — | 14 |
| REGN1909 | 16 | 18 | 20 | 22 | 24 | 26 | — | 28 |

TABLE 3
Nucleic Acid Sequence Identifiers

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| REGN1908 | 1 | 3 | 5 | 7 | 9 | 11 | — | 13 |
| REGN1909 | 15 | 17 | 19 | 21 | 23 | 25 | — | 27 |

### Example 2: Identification of Suitable Buffer, pH, and Excipients for Individual Anti-Fel d1 Antibodies

Initial formulation development activities involved screening for buffer and pH conditions that demonstrated a good balance of physical and chemical stability for each of the anti-Fel d1 antibodies REGN1908 and REGN1909. Anti-Fel D1 antibody (REGN1908 or REGN1909) at a concentration of 5 mg/mL was incubated in phosphate buffer at pH 6.0, 6.5, 7.0, 7.5, or 8.0, in histidine buffer at pH 5.5, 6.0, or 6.5, or acetate buffer at pH 4.5, 5.0, or 5.5. The formulations were subjected to agitation assay and evaluated for the formation of aggregates and charge variant species. 10 mM histidine was found to impart the best stability to both REGN1908 and REGN1909. In the agitation assay, REGN1908 formulations ≥pH 6.0 and REGN1909 formulations >pH 6.0 and <pH 5.5 failed the agitation studies. REGN1908 was determined to be the most stable with respect to aggregate formation at pH range 5.5-6.0 and with respect to charge variants at pH 5.0-6.0. REGN1909 was determined to be the most stable with respect to aggregate formation at pH range 6.0-6.5 and with respect to charge variants at pH 5.5. Thus, it was determined that a pH of 5.8 provided a good balance of physical and chemical stability for both molecules.

Formulations containing REGN1908 or REGN1909 at a concentration of 20 mg/mL in a 10 mM histidine buffer at pH 6.0 were assessed for thermal stability in the absence or presence of a cryoprotectant. It was determined that both antibodies were stable in formulations containing 5% (w/v) sucrose as the cryoprotectant, but an increase in the percentage of high molecular weight species was observed in control formulations lacking the sucrose cryoprotectant.

Formulations containing REGN1908 or REGN1909 were also screened for the effects of a surfactant on agitation stability and thermal stability. Agitation stress was applied to all formulations via vortexing at 1000 rpm for 120 minutes. Thermal stability was evaluated by incubating all formulations at an accelerated condition of 45° C. for 28 days. Polysorbate 20 (0.1% w/v), polysorbate 80 (0.1% w/v), PEG 3350 (0.5% w/v), and PEG 3350 (1.5% w/v) were all tested in formulations containing REGN1908 or REGN1909 at a concentration of 20 mg/mL, 5% (w/v) sucrose, and 10 mM histidine buffer at pH 6.0. All of the surfactants tested conferred agitation protection. In a thermal stability assay, it was found that REGN1908 had lower thermal stability with polysorbate 20 as compared with polysorbate 80 (thermal stability rank: 0.1% PS80>0.5% PEG>1.5% PEG>0.1% PS20). REGN1909 was thermally stable with all tested surfactants.

Based on these initial formulation development activities, it was determined that a formulation comprising 10 mM histidine, 5% (w/v) sucrose, and 0.1% (w/v) polysorbate 80 at pH 5.8 was suitable for formulating REGN1908 or REGN1909.

### Example 3: Formulation of Anti-Fel d1 Antibodies at High Concentration

Subsequent formulation development activities included formulating the anti-Fel d1 antibodies individually at higher concentrations, which is desirable for minimizing the volume of drug that is delivered to a patient. However, it is known in the art that as the concentration of antibody is increased, the viscosity of the formulation also increases. Higher viscosity in turn can lead to challenges in manufacturing and in patient administration.

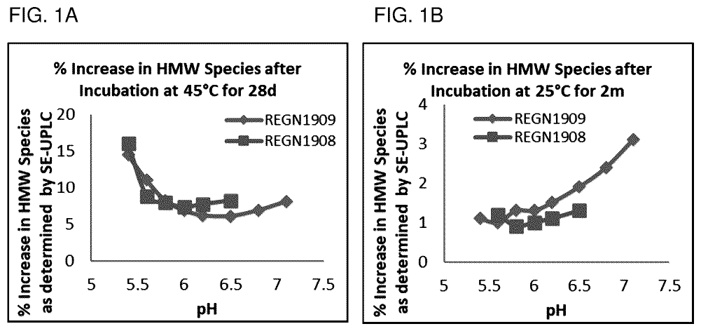

REGN1908 at a concentration of 150 mg/mL was formulated with 10 mM histidine at different pHs ranging from 5.4 to 6.5. The formulations were evaluated by SE-UPLC for the formation of aggregates under two different conditions: after incubation at 45° C. for 28 days and after incubation at 25° C. for 2 months. As shown in FIG. 1A, unfavorable levels of HMW aggregates were seen with the pH 5.4 formulation after incubation at 45° C. for 28 days. After incubation at 45° C. for 28 days, the lowest levels of HMW species were observed for the formulation at pH 6.0 (FIG. 1A), while after incubation at 25° C. for 2 months, the lowest levels of HMW species were observed for the formulations at pH 5.8, 6.0, and 6.2 (FIG. 1B).

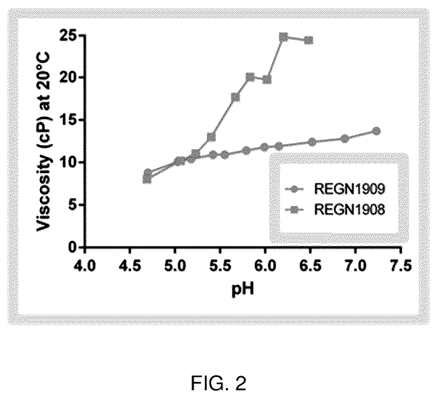

The REGN1908 formulations were also evaluated for viscosity at 20° C. As shown in FIG. 2, REGN1908 at 150 mg/mL and formulated at pH 5.4 had the lowest viscosity, while formulations at pH 6.2 or 6.5 had a viscosity >20 cP.

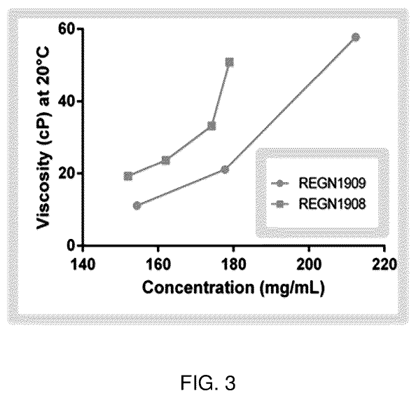

As shown in Table 4 below, formulating 150 mg/mL REGN1908 at a pH of 6.0 struck the best balance of acceptable viscosity and low HMW aggregate formation. Formulations at pH 6.0 were then tested to determine the effects of further increasing REGN1908 concentration on viscosity. As shown in FIG. 3, it was found that while 150 mg/mL REGN1908 exhibited a low viscosity of 19.7 cP, higher concentrations resulted in formulations having a viscosity >20 cP.

TABLE 4
Viscosity and Aggregation of REGN1908
High Concentration Formulations

| | | HMW Aggregation | HMW Aggregation |
|---|---|---|---|
| pH | Viscosity | 45° C., 28 days | 25° C., 2 months |
| 5.4 | + | − | NA |
| 5.6 | 0 | 0 | 0 |
| 5.8 | 0 | 0 | + |
| 6.0 | 0 | + | + |
| 6.2 | − | 0 | + |
| 6.5 | − | 0 | 0 |

+ favorable;
0 neutral;
− unfavorable;
NA not available

REGN1909 at a concentration of 150 mg/mL was formulated with 10 mM histidine at different pHs ranging from 5.4 to 6.8. The formulations were evaluated by SE-UPLC for the formation of aggregates under two different conditions: after incubation at 45° C. for 28 days and after incubation at 25° C. for 2 months. As shown in FIG. 1A, unfavorable levels of HMW aggregates were seen with the pH 5.4 formulation after incubation at 45° C. for 28 days. After incubation at 45° C. for 28 days, the lowest levels of HMW species were observed for the formulation at pH 6.2 and 6.5 (FIG. 1A). After incubation at 25° C. for 2 months, the lowest levels of HMW species were observed for the formulations at pH 5.4, 5.6, 5.8, and 6.0 (FIG. 1B).

The REGN1909 formulations at different pHs were evaluated for viscosity at 20° C. As shown in FIG. 2, REGN1908 at 150 mg/mL and formulated at pH 5.4 had the lowest viscosity, although all tested formulations exhibited a viscosity ≤20 cP.

As shown in Table 5 below, formulating 150 mg/mL REGN1909 at a pH of 5.6 to 6.0 struck the best balance of acceptable viscosity and low HMW aggregate formation. Formulations at pH 6.0 were then tested to determine the effects of further increasing REGN1909 concentration on viscosity. As shown in FIG. 3, it was found that 150 mg/mL REGN1909 exhibited a viscosity of 11.8 cP. Higher concentrations up to about 180 mg/mL still exhibited an acceptable viscosity.

TABLE 5
Viscosity and Aggregation of REGN1909
High Concentration Formulations

| | | HMW Aggregation | HMW Aggregation |
|---|---|---|---|
| pH | Viscosity | 45° C., 28 days | 25° C., 2 months |
| 5.4 | + | − | + |
| 5.6 | + | 0 | + |
| 5.8 | + | 0 | + |
| 6.0 | + | 0 | + |
| 6.2 | + | + | 0 |
| 6.5 | + | + | 0 |
| 6.8 | + | 0 | 0 |

+ favorable;
0 neutral;
− unfavorable

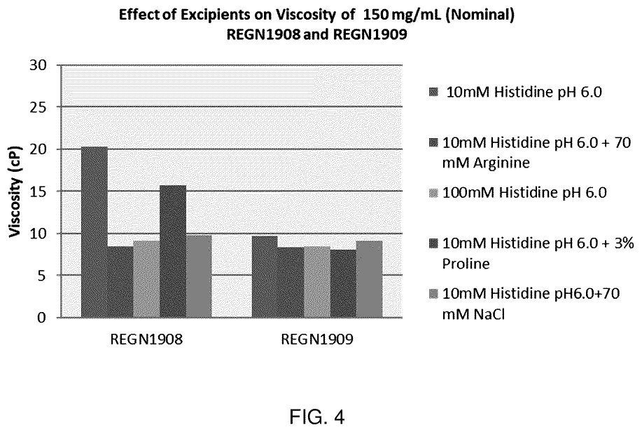

The effects of four different viscosity reducers on high-concentration formulations of REGN1908 or REGN1909 were also tested. REGN1908 or REGN1909 at 150 mg/mL was formulated at pH 6.0 and the following excipients: (i) 10 mM histidine (i.e., no viscosity reducer); (ii) 10 mM histidine+70 mM arginine-HCl; (iii) 100 mM histidine; (iv) 10 mM histidine+3% proline; or (v) 10 mM histidine+70 mM sodium chloride. The formulations were evaluated for viscosity at 20° C. As shown in FIG. 4 and in Table 6 below, 70 mM arginine was the most effective in reducing viscosity of REGN1908 (the more viscous of the two antibodies). For REGN1908, a viscosity <10 cP was obtained using 70 mM arginine, 100 mM histidine, or 70 mM sodium chloride as the viscosity reducer. For REGN1909 each of the viscosity reducers tested resulted in a formulation having a viscosity <10 cP.

TABLE 6
Effects of Viscosity Reducers

| | % cP reduction | % cP reduction |
|---|---|---|
| Formulation | for REGN1908 | for REGN1909 |
| 10 mM histidine, no reducer | 0% | 0% |
| 100 mM histidine | 55% | 13% |
| 10 mM histidine, 70 mM arginine | 60% | 15% |
| 10 mM histidine, 3% proline | 22.5% | 20% |
| 10 mM histidine, 70 mM NaCl | 52.5% | 4% |

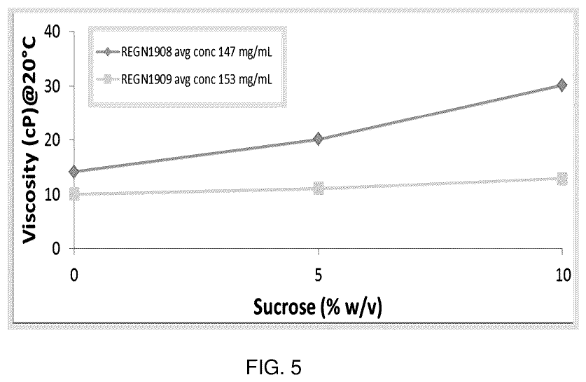

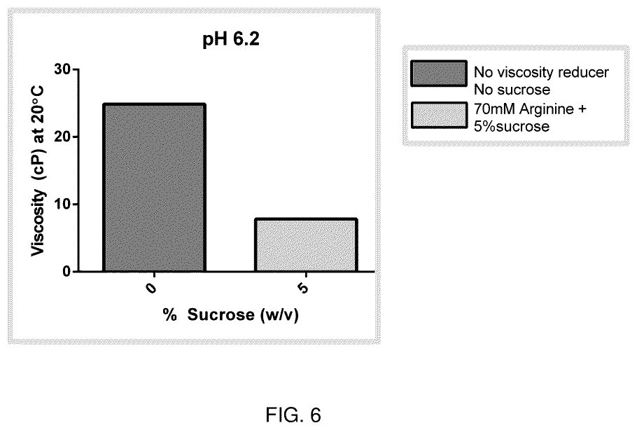

The effects of cryoprotectant on viscosity were tested for REGN1908 and REGN1909. REGN1908 or REGN1909 was formulated at a target concentration of 150 mg/mL with 10 mM histidine at pH 6.0. As shown in FIG. 5, the viscosity of REGN1909 was essentially unaffected by the addition of the cryoprotectant sucrose at 5% w/v or 10% w/v. However, the viscosity of REGN1908 was approximately 20 cP in the presence of 5% sucrose and increased to 30 cP in the presence of 10% sucrose. Further testing was performed with REGN1908 to determine the effects on viscosity of including 70 mM arginine-HCl in the presence of sucrose. As shown in FIG. 6, it was determined that 70 mM arginine-HCl was able to reduce the viscosity of a REGN1908 formulation at a concentration of ˜140 mg/mL with 5% sucrose w/v; the formulation with 70 mM arginine-HCl and 5% sucrose had a viscosity of about 8 cP at 20° C., versus a viscosity of about 24 cP in the absence of arginine and sucrose (FIG. 6) or a viscosity of about 20 cP in the presence of 5% sucrose (FIG. 5).

Table 7 below shows seven formulations each for REGN1908 and REGN1909 that were evaluated on stability at a target protein concentration of 175 mg/mL; this higher antibody concentration was chosen in case of potential need for high doses of the antibody or antibodies in clinical trials. As shown in Table 7 below, in the absence of a viscosity reducer or sucrose the REGN1908 formulation had a viscosity of 34.8 cP. Formulating REGN1908 with 10 mM histidine and 70 mM arginine, without sucrose or with 5% sucrose, resulted in formulations having the lowest viscosity (13.7 cP and 18.0 cP, respectively). Formulating REGN1908 with 80 mM histidine without sucrose resulted in a formulation having a viscosity of 15.8 cP; the addition of 5% or 10% sucrose increased the viscosity to 20.5 cP or 23.3 cP, respectively. For the REGN1909 formulations, all of the tested formulations had a viscosity ≤20 cP.

TABLE 7
Viscosity of 175 mg/mL Formulations
of REGN1908 or REGN1909

| | | | | REGN1908 | REGN1909 |
|---|---|---|---|---|---|
| | Histi- | | | Viscosity | Viscosity |
| Base | dine | Arginine | Sucrose | (cP) at | (cP) at |
| Formulation | (mM) | (mM) | (% w/v) | 20° C. | 20° C. |
| 175 mg/mL | 10 | — | — | 34.8 | 19.0 |
| REGN1908 or | 80 | — | — | 15.8 | 11.1 |
| REGN1909 | 80 | — | 5 | 20.5 | 15.8 |
| pH 6.0, 0.1% | 80 | — | 10 | 23.3 | 16.9 |
| polysorbate | 10 | 70 | — | 13.7 | 13.9 |
| 80 | 10 | 70 | 5 | 18.0 | 15.8* |
| | 10 | 70 | 10 | 21.1 | 14.9 |

*The measured concentration of REGN1909 for this sample was 183 mg/mL. For the other samples, the concentration was ~175 mg/mL.

### Example 4: Exemplary Single Antibody Formulations

In certain embodiments, anti-Fel d1 antibodies (e.g., REGN1908 and REGN1909) are individually formulated in an aqueous buffered formulation comprising: (a) from 5 mM to 20 mM histidine buffer, (b) from 60 mM to 130 mM arginine, and (c) from 50 to 200 mg anti-Fel d1 antibody, at pH 6.0±0.3. In some embodiments, the formulation further comprises from 2.5% to 7.5% w/v sucrose and/or from 0.01% to 0.5% w/v polysorbate.

In certain embodiments, anti-Fel d1 antibodies (e.g., REGN1908 and REGN1909) are individually formulated in an aqueous buffered formulation comprising: (a) from 5 mM to 20 mM histidine buffer, (b) from 2.5% to 7.5% w/v sucrose, (c) from 60 mM to 130 mM arginine, and (d) from 50 to 200 mg anti-Fel d1 antibody, at pH 6.0±0.3. In some embodiments, the formulation further comprises from 0.01% to 0.5% w/v polysorbate.

In one exemplary embodiment, an anti-Fel d1 antibody formulation comprises: (a) 10 mM±2 mM histidine, (b) 5% 1% w/v sucrose, (c) 70 mM±14 mM arginine, and (d) 190 mg/mL±10 mg/mL REGN1908, at pH 6.0±0.3.

In one exemplary embodiment, an anti-Fel d1 antibody formulation comprises: (a) 10 mM±2 mM histidine, (b) 5%±1% w/v sucrose, (c) 70 mM±14 mM arginine, (d) 0.15%±0.075% w/v polysorbate 80, and (e) 150 mg/mL±15 mg/mL REGN1908, at pH 6.0±0.3.

In one exemplary embodiment, an anti-Fel d1 antibody formulation comprises: (a) 10 mM±2 mM histidine, (b) 5%±1% w/v sucrose, (c) 70 mM±14 mM arginine, and (d) 190 mg/mL±10 mg/mL REGN1909, at pH 6.0±0.3.

In one exemplary embodiment, an anti-Fel d1 antibody formulation comprises: (a) 10 mM±2 mM histidine, (b) 5%±1% w/v sucrose, (c) 70 mM±14 mM arginine, (d) 0.15%±0.075% w/v polysorbate 80, and (e) 150 mg/mL±15 mg/mL REGN1909, at pH 6.0±0.3.

### Example 5: Stability Studies for REGN1908 Formulations

REGN1908 was formulated at a concentration of ˜150 mg/mL or ˜190 mg/mL for various long-term stability studies. “REGN1908 Formulation #1” contained 191.2 mg/ml REGN1908, 10 mM histidine, 70 mM arginine-HCl, and 5% (w/v) sucrose, at pH 6.0. “REGN1908 Formulation #2” contained 155.7 mg/ml REGN1908, 10 mM histidine, 70 mM arginine-HCl, 5% (w/v) sucrose, and 0.15% (w/v) super refined polysorbate 80, at pH 6.0. Stability studies on REGN1908 Formulation #1 and #2 were carried out in 5 mL polycarbonate vials.

### REGN1908 Formulation #1

For stability studies of REGN1908 Formulation #1, long term storage conditions were −80° C. or −30° C., for 0, 1, 3, 6, 9, 12, 18, and 24 months. Accelerated conditions were −20° C. for 0, 1, 3, and 6 months; 5° C. for 0, 0.5, 1, 3, and 6 months; or 25° C./60% RH for 0, 0.5, 1, 3, and 6 months. Thermal stress conditions were 40° C./75% RH, for 0, 0.25, 0.5, 1, and 3 months. Stability under physical stresses including agitation and freeze/thaw was also evaluated. For the agitation assays, samples were vortexed at 1000 RPM for 0, 5, and 10 minutes. For freeze/thaw assays, samples were frozen at −30° C. and thawed at room temperature for 0, 2, and 4 cycles.

12 months of storage stability data are available for REGN1908 Formulation #1. No appreciable changes in the physical or chemical stability of REGN1908 Formulation #1 were detected when stored at −80° C. and −30° C. for up to 24 months (Table 8 and Table 9). All attributes examined met quality target under storage conditions. These results indicate that REGN1908 Formulation #1 is stable for at least 24 months when frozen at either −30° C. or −80° C. storage condition.

The research stability studies indicated that the REGN1908 Formulation #1 was physically and chemically stable when incubated at −20° C. for up to 6 months, 5° C. for up to 6 months, and at 25° C./60% RH for up to 0.5 month (Table 10) with no meaningful changes observed in any monitored stability attributes. At 25° C./60% RH, 6 months of storage, a 1.1% increase in HMW species was observed by SE-UPLC and an increase of 8.8% in Region 1 (acidic variants) with no meaningful changes to Region 3 (basic variants) was observed by iCIEF.

Following incubation at 40° C./75% RH for 3 months, a 14.7% increase in HMW species was observed by SE-UPLC. An increase of 35.8% in Region 1 (acidic charge variants) was observed by iCIEF (Table 10). A concomitant decrease of 31.8% in Region 2 (main peak), with no meaningful change to Region 3 (basic charge variants) was observed by iCIEF.

REGN1908 Formulation #1 was chemically stable when vortexed for either 5 or 10 minutes (Table 11). However, after 5 and 10 minutes of an extreme stress condition of vortex agitation, the formulation experienced a 0.3% and 0.5% increase in HMW species respectively. REGN1908 Formulation #1 was physically and chemically stable when subjected to 4 freezing and thawing cycles (Table 11). No appreciable change in the physical or chemical stability was detected in any of the monitored attributes.

TABLE 8
Stability of REGN1908 Formulation #1 at −80° C.

| | | Length of storage (months) | | |
|---|---|---|---|---|
| Assay | | 0 | 12 | 24 |
| Visual appearance (pass as clear, | | Pass | Pass | Pass |
| colorless to pale yellow, essentially free | | | | |
| from visible particles) | | | | |
| pH | | 6.0 | 6.0 | 6.0 |
| Total protein | | 197 | 199.7 | 203.1 |
| (mg/mL) | | | | |
| Purity by SE- | % main | 98.5 | 98.4 | 98.5 |
| UPLC | % LMW species | 0.0 | 0.1 | 0.0 |
| | % HMW species | 1.5 | 1.6 | 1.5 |
| Charge variant | % acidic (Region 1) | 32.8 | 35.5 | 35.0 |
| analysis by iCIEF | % main (Region 2) | 54.1 | 51.0 | 52.2 |
| | % basic (Region 3) | 13.2 | 13.5 | 12.8 |
| Particulate matter | ≥2 to <10 μm | 576 | 1118 | 570 |
| (MFI) - unfiltered | (particle/mL) | | | |
| | ≥10 μm | 25 | 40 | 50 |
| | (particle/mL) | | | |
| | ≥25 μm | 2 | 0 | 3 |
| | (particle/mL) | | | |

NP = not performed

TABLE 9
Stability of REGN1908 Formulation #1 at −30° C.

| | | Length of storage (months) | | |
|---|---|---|---|---|
| Assay | | 0 | 12 | 24 |
| Visual appearance (pass as clear, | | Pass | Pass | Pass |
| colorless to pale yellow, essentially free | | | | |
| from visible particles) | | | | |
| pH | | 6.0 | 6.0 | 6.1 |
| Total protein | | 197 | 201.5 | 205.0 |
| (mg/mL) | | | | |
| Purity by SE- | % main | 98.5 | 98.4 | 98.5 |
| UPLC | % LMW species | 0.0 | 0.1 | 0.0 |
| | % HMW species | 1.5 | 1.6 | 1.5 |
| Charge variant | % acidic (Region 1) | 32.8 | 35.3 | 35.2 |
| analysis by iCIEF | % main (Region 2) | 54.1 | 50.9 | 51.6 |
| | % basic (Region 3) | 13.2 | 13.8 | 13.2 |
| Particulate matter | ≥2 to <10 μm | 576 | 2198 | 609 |
| (MFI) - unfiltered | (particle/mL) | | | |
| | ≥10 μm | 25 | 50 | 23 |
| | (particle/mL) | | | |
| | ≥25 μm | 2 | 6 | 3 |
| | (particle/mL) | | | |

NP = not performed

TABLE 10
Stability of REGN1908 Formulation #1 Incubated at −20° C., 5° C., 25° C./60% RH, and 40° C./75% RH

| | | Storage Condition | | | | | |
|---|---|---|---|---|---|---|---|
| | | | −20° C. | 5° C. | 25° C./60% RH | 40° C./75% RH | |
| | | No | storage | storage | storage | storage | |
| | | storage | (months) | (months) | (months) | (months) | |
| | | Assay | | | | | |
| | | 0 | 6 | 6 | 6 | 1 | 3 |
| Visual appearance (pass as clear, | | Pass | Pass | Pass | Pass | Pass | Fail |
| colorless to pale yellow, essentially | | | | | | | |
| free from visible particles) | | | | | | | |
| pH | | 6.0 | 6.0 | 6.0 | 6.0 | 6.0 | 6.1 |
| Total protein (mg/mL) | | 197 | 199.4 | 202.6 | 217 | 205 | 211.6 |
| Purity by SE-UPLC | % main | 98.5 | 98.4 | 98.0 | 97.4 | 95.5 | 83.5 |
| | % LMW species | 0.0 | 0.0 | 0.0 | 0.0 | 0.1 | 0.3 |
| | % HMW species | 1.5 | 1.6 | 2.0 | 2.6 | 4.4 | 16.2 |
| Charge variant | % acidic | 32.8 | 35.7 | 35.2 | 41.6 | 46.2 | 68.6 |
| analysis by iCIEF | (Region 1) | | | | | | |
| | % main | 54.1 | 50.8 | 51.9 | 44.3 | 40.5 | 22.3 |
| | (Region 2) | | | | | | |
| | % basic | 13.2 | 13.5 | 12.9 | 14.1 | 13.3 | 9.1 |
| | (Region 3) | | | | | | |
| Particulate | ≥2 to <10 μm | 576 | 4058 | 5740 | 35975 | NP | 128750 |
| matter (MFI) - | (particle/mL) | | | | | | |
| unfiltered | ≥10 μm | 25 | 288 | 440 | 1647 | NP | 10921 |
| | (particle/mL) | | | | | | |
| | ≥25 μm | 2 | 46 | 71 | 54 | NP | 811 |
| | (particle/mL) | | | | | | |

NP = not performed

TABLE 11
Stability of REGN1908 Formulation #1 - Effect of Agitation and Freeze/Thaw

| | | Storage Condition | | | | |
|---|---|---|---|---|---|---|
| | | No | Agitation | | Freeze/Thaw | |
| | | Stress | (minutes) | | (cycles) | |
| | | Assay | | | | |
| | | 0 | 5 | 10 | 2 | 4 |
| Visual appearance (pass as clear, | | Pass | Pass | Pass | Pass | Pass |
| colorless to pale yellow, essentially | | | | | | |
| free from visible particles) | | | | | | |
| pH | | 6.0 | 6.0 | 6.0 | 6.0 | 6.0 |
| Total protein (mg/mL) | | 197 | 191.9 | 198.1 | 202.3 | 197 |
| Purity by SE-UPLC | % main | 98.5 | 98.2 | 98.0 | 98.5 | 98.5 |
| | % LMW species | 0.0 | 0.0 | 0.0 | 0.0 | 0.0 |
| | % HMW species | 1.5 | 1.8 | 2.0 | 1.5 | 1.5 |
| Charge variant | % acidic (Region 1) | 32.8 | 35.6 | 35.8 | 34.2 | 32.0 |
| analysis by iCIEF | % main (Region 2) | 54.1 | 51.0 | 50.7 | 52.5 | 54.4 |
| | % basic (Region 3) | 13.2 | 13.5 | 13.5 | 13.3 | 13.7 |
| Particulate matter | ≥2 to <10 μm | 576 | NP | 572 | NP | 2697 |
| (MFI) - unfiltered | (particle/mL) | | | | | |
| | ≥10 μm | 25 | NP | 50 | NP | 163 |
| | (particle/mL) | | | | | |
| | ≥25 μm | 2 | NP | 10 | NP | 17 |
| | (particle/mL) | | | | | |

NP = not performed

### REGN1908 Formulation #2

For stability studies of REGN1908 Formulation #2, long term storage conditions were −80° C. or −30° C., for 0, 1, 3, 6, 9, 12, 18, and 24 months. Accelerated conditions were −20° C. for 0, 1, 3, and 6 months; 5° C. for 0, 0.5, 1, 3, and 6 months; or 25° C./60% RH for 0, 0.5, 1, 3, and 6 months. Thermal stress conditions were 40° C./75% RH, for 0, 0.25, 0.5, 1, and 3 months. Stability under physical stresses including agitation and freeze/thaw was also evaluated. For the agitation assays, samples were vortexed at 1000 RPM for 0, 30, and 60 minutes. For freeze/thaw assays, samples were frozen at −30° C. and thawed at room temperature for 0, 2, and 4 cycles.

24 months of storage stability data are available for REGN1908 Formulation #2. No appreciable changes in the physical or chemical stability of REGN1908 Formulation #2 were detected when stored at −80° C. and −30° C. for up to 24 months (Table 12 and Table 13). All attributes examined met quality target under storage conditions. These results indicate that REGN1908 Formulation #2 is stable for at least 24 months when frozen at storage conditions.

The research stability studies indicated that the REGN1908 Formulation #2 was physically and chemically stable when incubated at −20° C. for up to 6 months, 5° C. for up to 6 months, and at 25° C./60% RH for up to 0.5 month (Table 14) with no meaningful changes observed in any monitored stability attributes. At 25° C./60% RH at 6 months of storage a 0.8% increase in HMW species was observed by SE-UPLC and an increase of 7.6% Region 1 (acidic variants) with no meaningful changes to Region 3 (basic variants) observed by iCIEF.

Following incubation at 40° C./75% RH for 3 months, a 13.1% increase in HMW species was observed by SE-UPLC. An increase of 34.8% in Region 1 (acidic charge variants) was observed by iCIEF (Table 14). A concomitant decrease of 31.1% in Region 2 (main peak), with no meaningful change to Region 3 (basic charge variants) was observed by iCIEF.

REGN1908 Formulation #2 was physically and chemically stable when vortexed for either 60 or 120 minutes (Table 15). No appreciable change in the physical or chemical stability was detected in any of the monitored attributes. REGN1908 Formulation #2 was physically and chemically stable when subjected to up to 4 freezing and thawing cycles (Table 15). No appreciable change in the physical or chemical stability was detected in any of the monitored attributes.

TABLE 12
Stability of REGN1908 Formulation #2 at −80° C.

| | | Length of storage (months) | | |
|---|---|---|---|---|
| Assay | | 0 | 12 | 24 |
| Visual appearance (pass as clear, | | Pass | Pass | Pass |
| colorless to pale yellow, essentially free | | | | |
| from visible particles) | | | | |
| pH | | 6.0 | 6.04 | 6.0 |
| Total protein | | 154.6 | 158 | 153.6 |
| (mg/mL) | | | | |
| Purity by SE-UPLC | % main | 98.5 | 98.6 | 98.6 |
| | % LMW species | 0.0 | 0.0 | 0.0 |
| | % HMW species | 1.5 | 1.4 | 1.4 |
| Charge variant | % acidic (Region 1) | 33.5 | 34.0 | 34.2 |
| analysis by iCIEF | % main (Region 2) | 53.3 | 52.9 | 52.6 |
| | % basic (Region 3) | 13.2 | 13.1 | 13.2 |
| Particulate matter | ≥2 to <10 μm | 177 | 179 | 186 |
| (MFI) - unfiltered | (particle/mL) | | | |
| | ≥10 μm | 21 | 19 | 11 |
| | (particle/mL) | | | |
| | ≥25 μm | 0 | 2 | 0 |
| | (particle/mL) | | | |

TABLE 13
Stability of REGN1908 Formulation #2 at −30° C.

| | | Length of storage (months) | | |
|---|---|---|---|---|
| Assay | | 0 | 12 | 24 |
| Visual appearance (pass as clear, | | Pass | Pass | Pass |
| colorless to pale yellow, essentially free | | | | |
| from visible particles) | | | | |
| pH | | 6.0 | 6.0 | 6.0 |
| Total protein | | 154.6 | 160.9 | 154.9 |
| (mg/mL) | | | | |
| Purity by SE-UPLC | % main | 98.5 | 98.5 | 98.6 |
| | % LMW species | 0.0 | 0.0 | 0.0 |
| | % HMW species | 1.5 | 1.4 | 1.4 |
| Charge variant | % acidic (Region 1) | 33.5 | 35.6 | 34.7 |
| analysis by iCIEF | % main (Region 2) | 53.3 | 51.1 | 52.1 |
| | % basic (Region 3) | 13.2 | 13.3 | 13.2 |
| Particulate matter | ≥2 to <10 μm | 177 | 313 | 82 |
| (MFI) - unfiltered | (particle/mL) | | | |
| | ≥10 μm | 21 | 23 | 9 |
| | (particle/mL) | | | |
| | ≥25 μm | 0 | 4 | 5 |
| | (particle/mL) | | | |

TABLE 14
Stability of REGN1908 Formulation #2 Incubated at −20° C., 5° C., 25° C./60% RH, and 40° C./75% RH

| | | Storage Condition | | | | | |
|---|---|---|---|---|---|---|---|
| | | | −20° C. | 5° C. | 25° C./60% RH | 40° C./75% RH | |
| | | No | storage | storage | storage | storage | |
| | | storage | (months) | (months) | (months) | (months) | |
| | | Assay | | | | | |
| | | 0 | 6 | 6 | 6 | 1 | 3 |
| Visual appearance (pass as clear, | | Pass | Pass | Pass | Pass | Pass | Pass |
| colorless to pale yellow, essentially | | | | | | | |
| free from visible particles) | | | | | | | |
| pH | | 6.0 | 6.0 | 6.1 | 6.0 | 6.0 | 6.1 |
| Total protein (mg/mL) | | 154.6 | 155.7 | 160.8 | 172.8 | 160.2 | 160.8 |
| Purity by SE-UPLC | % main | 98.5 | 98.5 | 98.2 | 97.7 | 96.0 | 85.1 |
| | % LMW species | 0.0 | 0.0 | 0.0 | 0.0 | 0.1 | 0.3 |
| | % HMW species | 1.5 | 1.5 | 1.8 | 2.3 | 4.0 | 14.6 |
| Charge variant | % acidic | 33.5 | 35.1 | 34.7 | 41.1 | 47.4 | 68.3 |
| analysis by iCIEF | (Region 1) | | | | | | |
| | % main | 53.3 | 51.8 | 52.2 | 45.2 | 39.6 | 22.2 |
| | (Region 2) | | | | | | |
| | % basic | 13.2 | 13.1 | 13.2 | 13.8 | 13.1 | 9.6 |
| | (Region 3) | | | | | | |
| Particulate | ≥2 to <10 μm | 177 | 1303 | 8720 | 9059 | NP | 4510 |
| matter (MFI) - | (particle/mL) | | | | | | |
| unfiltered | ≥10 μm | 21 | 100 | 1022 | 1045 | NP | 442 |
| | (particle/mL) | | | | | | |
| | ≥25 μm | 0 | 19 | 186 | 227 | NP | 100 |
| | (particle/mL) | | | | | | |

NP = not performed

TABLE 15
Stability of REGN1908 Formulation #2 - Effect of Agitation and Freeze/Thaw

| | | Storage Condition | | | | |
|---|---|---|---|---|---|---|
| | | No | Agitation | | Freeze/Thaw | |
| | | Stress | (minutes) | | (cycles) | |
| | | Assay | | | | |
| | | 0 | 60 | 120 | 2 | 4 |
| Visual appearance (pass as clear, | | Pass | Pass | Pass | Pass | Pass |
| colorless to pale yellow, essentially | | | | | | |
| free from visible particles) | | | | | | |
| pH | | 6.0 | 6.0 | 6.0 | 6.0 | 6.0 |
| Total protein (mg/mL) | | 154.6 | 152.3 | 152.5 | 153.3 | 153.5 |
| Purity by SE-UPLC | % main | 98.5 | 98.6 | 98.6 | 98.6 | 98.6 |
| | % LMW species | 0.0 | 0.0 | 0.0 | 0.0 | 0.0 |
| | % HMW species | 1.5 | 1.5 | 1.4 | 1.4 | 1.4 |
| Charge variant | % acidic (Region 1) | 33.5 | 33.5 | 33.0 | 35.1 | 35.6 |
| analysis by iCIEF | % main (Region 2) | 53.3 | 53.3 | 53.6 | 51.9 | 50.9 |
| | % basic (Region 3) | 13.2 | 13.2 | 13.3 | 13.0 | 13.6 |
| Particulate | ≥2 to <10 μm | 177 | NP | 288 | NP | 5105 |
| matter (MFI) | (particle/mL) | | | | | |
| | ≥10 μm | 21 | NP | 21 | NP | 540 |
| | (particle/mL) | | | | | |
| | ≥25 μm | 0 | NP | 2 | NP | 75 |
| | (particle/mL) | | | | | |

NP = not performed

### Example 6: Stability Studies for REGN1909 Formulations

REGN1909 was formulated at a concentration of ˜150 mg/mL or ˜190 mg/mL for various long-term stability studies. “REGN1909 Formulation #1” contained 189.4 mg/ml REGN1909, 10 mM histidine, 70 mM arginine-HCl, and 5% (w/v) sucrose, at pH 6.0. “REGN1909 Formulation #2” contained 152.6 mg/ml REGN1909, 10 mM histidine, 70 mM arginine-HCl, 5% (w/v) sucrose, and 0.15% (w/v) super refined polysorbate 80, at pH 6.0. Stability studies on REGN1909 Formulation #1 and #2 were carried out in 5 mL polycarbonate vials.

### REGN1909 Formulation #1

For stability studies of REGN1909 Formulation #1, long term storage conditions were −80° C. or −30° C., for 0, 1, 3, 6, 9, 12, 18, and 24 months. Accelerated conditions were −20° C. for 0, 1, 3, and 6 months; 5° C. for 0, 0.5, 1, 3, and 6 months; or 25° C./60% RH for 0, 0.5, 1, 3, and 6 months. Thermal stress conditions were 40° C./75% RH, for 0, 0.25, 0.5, 1, and 3 months. Stability under physical stresses including agitation and freeze/thaw was also evaluated. For the agitation assays, samples were vortexed at 1000 RPM for 0, 5, and 10 minutes. For freeze/thaw assays, samples were frozen at −30° C. and thawed at room temperature for 0, 2, and 4 cycles.

24 months of storage stability data are available for REGN1909 Formulation #1. No appreciable changes in the physical or chemical stability of REGN1909 Formulation #1 were detected when stored at −80° C. and −30° C. for up to 24 months (Table 16 and Table 17). All attributes examined met quality target under storage conditions. These results indicate that REGN1909 Formulation #1 is stable for at least 24 months when frozen at either −30° C. or −80° C. storage condition.

The research stability studies indicated that the REGN1909 Formulation #1 was physically and chemically stable when incubated at −20° C. for up to 6 months, 5° C. for up to 6 months, and at 25° C./60% RH for up to 0.5 month (Table 18) with no meaningful changes observed in any monitored stability attributes. At 25° C./60% RH, 6 months of storage, a 1.8% increase in HMW species was observed by SE-UPLC and an increase of 8.8% in Region 1 (acidic variants) with no meaningful changes to Region 3 (basic variants) was observed by iCIEF.

Following incubation at 40° C./75% RH for 3 months, a 6.4% increase in HMW species was observed by SE-UPLC. An increase of 28.4% in Region 1 (acidic charge variants) was observed by iCIEF (Table 18). A concomitant decrease of 28.8% in Region 2 (main peak), with no meaningful change to Region 3 (basic charge variants) was observed by iCIEF.

REGN1909 Formulation #1 was physically and chemically stable when vortexed for 5 minutes (Table 19). However, after 10 minutes of an extreme stress of vortex agitation, REGN1909 Formulation #1 was chemically stable but not physically stable since there was a 0.3% increase in HMW species. REGN1909 Formulation #1 was physically and chemically stable when subjected to 4 freezing and thawing cycles (Table 19). No appreciable change in the physical or chemical stability was detected in any of the monitored attributes.

TABLE 16
Stability of REGN1909 Formulation #1 at −80° C.

| | | Length of storage (months) | | |
|---|---|---|---|---|
| Assay | | 0 | 12 | 24 |
| Visual appearance (pass as clear, | | Pass | Pass | Pass |
| colorless to pale yellow, essentially free | | | | |
| from visible particles) | | | | |
| pH | | 6.0 | 6.0 | 5.9 |
| Total protein | | 196.0 | 200.3 | 195.8 |
| (mg/mL) | | | | |
| Purity by SE-UPLC | % main | 98.5 | 98.6 | 98.6 |
| | % LMW species | 0.0 | 0.1 | 0.0 |
| | % HMW species | 1.5 | 1.4 | 1.4 |
| Charge variant | % acidic (Region 1) | 38.5 | 40.1 | 40.7 |
| analysis by iCIEF | % main (Region 2) | 53.6 | 52.9 | 52.4 |
| | % basic (Region 3) | 7.9 | 7.0 | 6.9 |
| Particulate matter | ≥2 to <10 μm | 964 | 1560 | 201 |
| (MFI) - unfiltered | (particle/mL) | | | |
| | ≥10 μm | 31 | 77 | 3 |
| | (particle/mL) | | | |
| | ≥25 μm | 4 | 15 | 0 |
| | (particle/mL) | | | |

TABLE 17
Stability of REGN1909 Formulation #1 at −30° C.

| | | Length of storage (months) | | |
|---|---|---|---|---|
| Assay | | 0 | 12 | 24 |
| Visual appearance (pass as clear, | | Pass | Pass | Pass |
| colorless to pale yellow, essentially free | | | | |
| from visible particles) | | | | |
| pH | | 6.0 | 6.0 | 5.9 |
| Total protein | | 196.0 | 206.1 | 199.1 |
| (mg/mL) | | | | |
| Purity by SE-UPLC | % main | 98.5 | 98.6 | 98.4 |
| | % LMW species | 0.0 | 0.0 | 0.0 |
| | % HMW species | 1.5 | 1.4 | 1.6 |
| Charge variant | % acidic (Region 1) | 38.5 | 39.8 | 41.5 |
| analysis by iCIEF | % main (Region 2) | 53.6 | 53.0 | 51.5 |
| | % basic (Region 3) | 7.9 | 7.2 | 7.1 |
| Particulate matter | ≥2 to <10 μm | 964 | 5403 | 159 |
| (MFI) - unfiltered | (particle/mL) | | | |
| | ≥10 μm | 31 | 177 | 11 |
| | (particle/mL) | | | |
| | ≥25 μm | 4 | 29 | 7 |
| | (particle/mL) | | | |

TABLE 18
Stability of REGN1909 Formulation #1 Incubated at −20° C., 5° C., 25° C./60% RH, and 40° C./75% RH

| | | Storage Condition | | | | | |
|---|---|---|---|---|---|---|---|
| | | | −20° C. | 5° C. | 25° C./60% RH | 40° C./75% RH | |
| | | No | storage | storage | storage | storage | |
| | | storage | (months) | (months) | (months) | (months) | |
| | | Assay | | | | | |
| | | 0 | 6 | 6 | 6 | 1 | 3 |
| Visual appearance (pass as clear, | | Pass | Pass | Pass | Pass | Pass | Pass |
| colorless to pale yellow, essentially | | | | | | | |
| free from visible particles) | | | | | | | |
| pH | | 6.0 | 6.0 | 6.0 | 5.9 | 6.0 | 6.0 |
| Total protein (mg/mL) | | 196.0 | 198.3 | 197.4 | 219.3 | 189.4 | 201.8 |
| Purity by SE-UPLC | % main | 98.5 | 98.6 | 98.1 | 96.6 | 95.8 | 91.9 |
| | % LMW species | 0.0 | 0.0 | 0.0 | 0.1 | 0.1 | 0.3 |
| | % HMW species | 1.5 | 1.4 | 2.0 | 3.3 | 4.2 | 7.9 |
| Charge variant | % acidic | 38.5 | 39.4 | 38.6 | 47.3 | 51.9 | 66.9 |
| analysis by iCIEF | (Region 1) | | | | | | |
| | % main | 53.6 | 53.7 | 53.3 | 43.2 | 38.9 | 24.8 |
| | (Region 2) | | | | | | |
| | % basic | 7.9 | 6.9 | 8.2 | 9.5 | 9.2 | 8.3 |
| | (Region 3) | | | | | | |
| Particulate | ≥2 to <10 μm | 964 | 785 | 267 | 41100 | NP | 12350 |
| matter (MFI) - | (particle/mL) | | | | | | |
| unfiltered | ≥10 μm | 31 | 54 | 29 | 9062 | NP | 809 |
| | (particle/mL) | | | | | | |
| | ≥25 μm | 4 | 6 | 2 | 23 | NP | 13 |
| | (particle/mL) | | | | | | |

NP = not performed

TABLE 19
Stability of REGN1909 Formulation #1 - Effect of Agitation and Freeze/Thaw

| | | Storage Condition | | | | |
|---|---|---|---|---|---|---|
| | | No | Agitation | | Freeze/Thaw | |
| | | Stress | (minutes) | | (cycles) | |
| | | Assay | | | | |
| | | 0 | 5 | 10 | 2 | 4 |
| Visual appearance (pass as clear, | | Pass | Pass | Pass | Pass | Pass |
| colorless to pale yellow, essentially | | | | | | |
| free from visible particles) | | | | | | |
| pH | | 6.0 | 6.1 | 6.1 | 6.0 | 6.1 |
| Total protein (mg/mL) | | 196.0 | 198.4 | 188.1 | 191.1 | 194.7 |
| Purity by SE-UPLC | % main | 98.5 | 98.4 | 98.2 | 98.6 | 98.6 |
| | % LMW species | 0.0 | 0.0 | 0.0 | 0.0 | 0.0 |
| | % HMW species | 1.5 | 1.6 | 1.8 | 1.4 | 1.4 |
| Charge variant | % acidic (Region 1) | 38.5 | 36.8 | 39.1 | 38.7 | 38.8 |
| analysis by iCIEF | % main (Region 2) | 53.6 | 56.0 | 52.7 | 54.4 | 54.1 |
| | % basic (Region 3) | 7.9 | 7.1 | 8.2 | 6.9 | 71 |
| Particulate | ≥2 to <10 μm | 964 | NP | 1558 | NP | 1243 |
| matter (MFI) | (particle/mL) | | | | | |
| | ≥10 μm | 31 | NP | 60 | NP | 61 |
| | (particle/mL) | | | | | |
| | ≥25 μm | 4 | NP | 4 | NP | 6 |
| | (particle/mL) | | | | | |

NP = not performed

### REGN1909 Formulation #2

For stability studies of REGN1909 Formulation #2, long term storage conditions were −80° C. or −30° C., for 0, 1, 3, 6, 9, 12, 18, and 24 months. Accelerated conditions were −20° C. for 0, 1, 3, and 6 months; 5° C. for 0, 0.5, 1, 3, and 6 months; or 25° C./60% RH for 0, 0.5, 1, 3, and 6 months. Thermal stress conditions were 40° C./75% RH, for 0, 0.25, 0.5, 1, and 3 months. Stability under physical stresses including agitation and freeze/thaw was also evaluated. For the agitation assays, samples were vortexed at 1000 RPM for 0, 30, and 60 minutes. For freeze/thaw assays, samples were frozen at −30° C. and thawed at room temperature for 0, 2, and 4 cycles.

24 months of storage stability data are available for REGN1909 Formulation #2. No appreciable changes in the physical or chemical stability of REGN1909 Formulation #2 were detected when stored at 80° C. and −30° C. for up to 24 months (Table 20 and Table 21). These results indicate that REGN1909 Formulation #2 is stable for at least 24 months when stored frozen at either −30° C. or −80° C. storage condition.

Research stability studies indicated that the REGN1909 Formulation #2 was physically and chemically stable when incubated at −20° C. for up to 6 months, 5° C. for up to 6 months, and at 25° C./60% RH for up to 0.5 month (Table 22) with no meaningful changes observed in any monitored stability attributes. At 25° C./60% RH at 6 months of storage a 1.5% increase in HMW species was observed by SE-UPLC and an increase of 11.0% Region 1 (acidic variants) with no meaningful changes to Region 3 (basic variants) was observed by iCIEF.

Following incubation at 40° C./75% RH for 3 months, a 5.4% increase in HMW species was observed by SE-UPLC. An increase of 31.3% in Region 1 (acidic charge variants) was observed by iCIEF (Table 22). A concomitant decrease of 29.6% in Region 2 (main peak), with no meaningful change to Region 3 (basic charge variants) was observed by iCIEF.

REGN1909 Formulation #2 was physically and chemically stable when vortexed for either 60 or 120 minutes (Table 23). Higher than normal sub-visible particulates were observed after 120 minutes of vortexing. However, no increase in % HMW was observed. A more extensive agitation of Formulation #2 was performed using an orbital shaker for 24 and 48 hours. After 48 hours on the orbital shaker, a 0.1% increase in HMW species was observed with low sub-visible particulates in the 10 and 25 μm bin ranges (255 and 21 particles/mL respectively). No appreciable change in the physical or chemical stability was detected in any of the other monitored attributes. REGN1909 Formulation #2 was physically and chemically stable when subjected to up to 4 freezing and thawing cycles (Table 23). No appreciable change in the physical or chemical stability was detected in any of the monitored attributes.

TABLE 20
Stability of REGN1909 Formulation #2 at −80° C.

| | | Length of storage (months) | | |
|---|---|---|---|---|
| Assay | | 0 | 12 | 24 |
| Visual appearance (pass as clear, | | Pass | Pass | Pass |
| colorless to pale yellow, essentially free | | | | |
| from visible particles) | | | | |
| pH | | 6.0 | 6.0 | 5.9 |
| Total protein | | 152.6 | 148.4 | 152.1 |
| (mg/mL) | | | | |
| Purity by SE-UPLC | % main | 98.7 | 98.7 | 98.6 |
| | % LMW species | 0.0 | 0.0 | 0.1 |
| | % HMW species | 1.3 | 1.3 | 1.3 |
| Charge variant | % acidic (Region 1) | 35.9 | 41.3 | 41.0 |
| analysis by iCIEF | % main (Region 2) | 56.0 | 51.1 | 51.8 |
| | % basic (Region 3) | 8.0 | 7.6 | 7.2 |
| Particulate matter | ≥2 to <10 μm | 11 | 819 | 73 |
| (MFI) - unfiltered | (particle/mL) | | | |
| | ≥10 μm | 4 | 54 | 3 |
| | (particle/mL) | | | |
| | ≥25 μm | 0 | 4 | 0 |
| | (particle/mL) | | | |

TABLE 21
Stability of REGN1909 Formulation #2 at −30° C.

| | | Length of storage (months) | | |
|---|---|---|---|---|
| Assay | | 0 | 12 | 24 |
| Visual appearance (pass as clear, | | Pass | Pass | Pass |
| colorless to pale yellow, essentially free | | | | |
| from visible particles) | | | | |
| pH | | 6.0 | 6.0 | 5.9 |
| Total protein | | 152.6 | 150.9 | 148.7 |
| (mg/mL) | | | | |
| Purity by SE-UPLC | % main | 98.7 | 98.7 | 98.6 |
| | % LMW species | 0.0 | 0.0 | 0.1 |
| | % HMW species | 1.3 | 1.3 | 1.3 |
| Charge variant | % acidic (Region 1) | 35.9 | 40.0 | 40.3 |
| analysis by iCIEF | % main (Region 2) | 56.0 | 52.7 | 52.6 |
| | % basic (Region 3) | 8.0 | 7.3 | 7.1 |
| Particulate matter | ≥2 to <10 μm | 11 | 190 | 234 |
| (MFI) - unfiltered | (particle/mL) | | | |
| | ≥10 μm | 4 | 19 | 19 |
| | (particle/mL) | | | |
| | ≥25 μm | 0 | 6 | 3 |
| | (particle/mL) | | | |

TABLE 22
Stability of REGN1909 Formulation #2 Incubated at −20° C., 5° C., 25° C./60% RH, and 40° C./75% RH

| | | Storage Condition | | | | | |
|---|---|---|---|---|---|---|---|
| | | | −20° C. | 5° C. | 25° C./60% RH | 40° C./75% RH | |
| | | No | storage | storage | storage | storage | |
| | | storage | (months) | (months) | (months) | (months) | |
| | | Assay | | | | | |
| | | 0 | 6 | 6 | 6 | 1 | 3 |
| Visual appearance (pass as clear, | | Pass | Pass | Pass | Pass | Pass | Pass |
| colorless to pale yellow, essentially | | | | | | | |
| free from visible particles) | | | | | | | |
| pH | | 6.0 | 6.0 | 6.0 | 6.0 | 6.0 | 6.0 |
| Total protein (mg/mL) | | 152.6 | 151.5 | 151.6 | 169.0 | 150.6 | 156.4 |
| Purity by SE-UPLC | % main | 98.7 | 98.7 | 98.3 | 97.1 | 96.4 | 92.9 |
| | % LMW species | 0.0 | 0.0 | 0.0 | 0.1 | 0.1 | 0.4 |
| | % HMW species | 1.3 | 1.3 | 1.8 | 2.8 | 3.5 | 6.7 |
| Charge variant | % acidic | 35.9 | 40.2 | 40.0 | 46.9 | 49.3 | 67.2 |
| analysis by iCIEF | (Region 1) | | | | | | |
| | % main | 56.0 | 51.9 | 52.3 | 44.3 | 41.9 | 26.4 |
| | (Region 2) | | | | | | |
| | % basic | 8.0 | 7.8 | 7.7 | 8.7 | 8.8 | 6.4 |
| | (Region 3 | | | | | | |
| Particulate | ≥2 to <10 μm | 11 | 951 | 3095 | 1500 | NP | 361 |
| matter (MFI) - | (particle/mL) | | | | | | |
| unfiltered | ≥10 μm | 4 | 86 | 378 | 161 | NP | 54 |
| | (particle/mL) | | | | | | |
| | ≥25 μm | 0 | 13 | 46 | 29 | NP | 15 |
| | (particle/mL) | | | | | | |

NP = not performed

TABLE 23
Stability of REGN1909 Formulation #2 - Effect of Agitation and Freeze/Thaw

| | | Storage Condition | | | | |
|---|---|---|---|---|---|---|
| | | No | Agitation | | Freeze/Thaw | |
| | | Stress | (minutes) | | (cycles) | |
| | | Assay | | | | |
| | | 0 | 60 | 120 | 2 | 4 |
| Visual appearance (pass as clear, | | Pass | Pass | Pass | Pass | Pass |
| colorless to pale yellow, essentially | | | | | | |
| free from visible particles) | | | | | | |
| pH | | 6.0 | 5.9 | 5.9 | 6.0 | 6.0 |
| Total protein (mg/mL) | | 152.6 | 149.8 | 153.0 | 148.2 | 151.3 |
| Purity by SE-UPLC | % main | 98.7 | 98.7 | 98.7 | 98.7 | 98.7 |
| | % LMW species | 0.0 | 0.0 | 0.0 | 0.0 | 0.0 |
| | % HMW species | 1.3 | 1.3 | 1.3 | 1.3 | 1.3 |
| Charge variant | % acidic (Region 1) | 35.9 | 35.6 | 36.1 | 36.3 | 36.4 |
| analysis by iCIEF | % main (Region 2) | 56.0 | 56.7 | 56.4 | 56.6 | 56.0 |
| | % basic (Region 3) | 8.0 | 7.7 | 7.4 | 7.2 | 7.6 |
| Particulate | ≥2 to <10 μm | 11 | NP | 6997 | NP | 1104 |
| matter (MFI) | (particle/mL) | | | | | |
| | ≥10 μm | 4 | NP | 730 | NP | 129 |
| | (particle/mL) | | | | | |
| | ≥25 μm | 0 | NP | 111 | NP | 15 |
| | (particle/mL) | | | | | |

NP = not performed

### Example 7: Co-Formulation of Anti-Fel d1 Antibodies

In circumstances in which multiple antibodies are to be administered concurrently to a patient, it can be beneficial to co-formulate the antibodies together in a single formulation (e.g., for ease of administration to the patient and enhanced patient compliance). Development activities were initiated for the co-formulation of a fixed dose combination of REGN1908 and REGN1909. For co-formulating REGN1908 and REGN1909, histidine buffer at a pH of 6.0 was selected; as shown in Tables 4 and 5 above, pH 6.0 was the optimal pH for achieving both acceptable viscosity and low or low/intermediate aggregation stability of both REGN1908 and REGN1909.

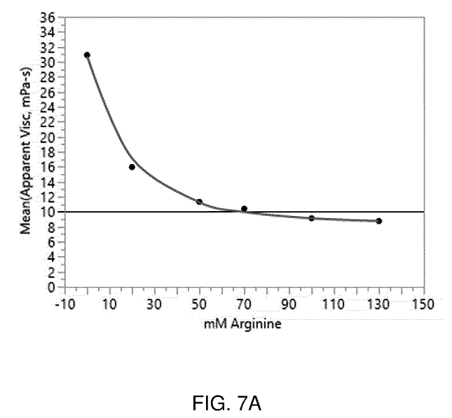

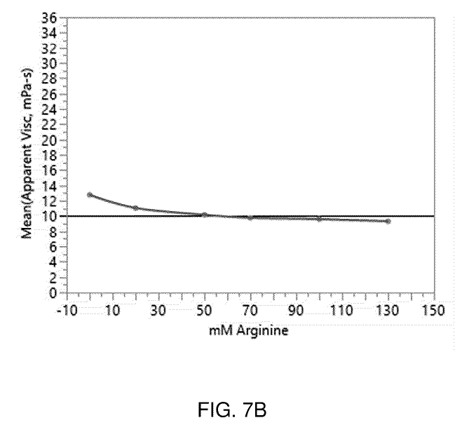

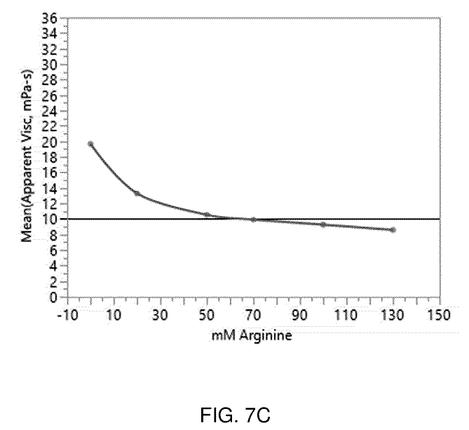

As disclosed in Example 3 above, it was found that REGN1908 was more viscous than REGN1909 in otherwise identical formulations. Formulations comprising both REGN1908 and REGN1909 were tested to determine whether co-formulation of REGN1908 and REGN1909 increased the formulation viscosity. Formulations containing a target total antibody concentration of 150 mg/mL (about 75 mg/mL REGN1908 and about 75 mg/mL REGN1909) were formulated at pH 6.1 with 10 mM histidine and 5% sucrose, and either no arginine or 20, 50, 70, 100, or 130 mM arginine. Co-formulations having at least 50 mM arginine had a similar viscosity as corresponding separately formulated REGN1908 or REGN1909 (FIG. 7A-7C). Significant differences in viscosity were observed in formulations without arginine or with 20 mM arginine (FIG. 7A-7C).

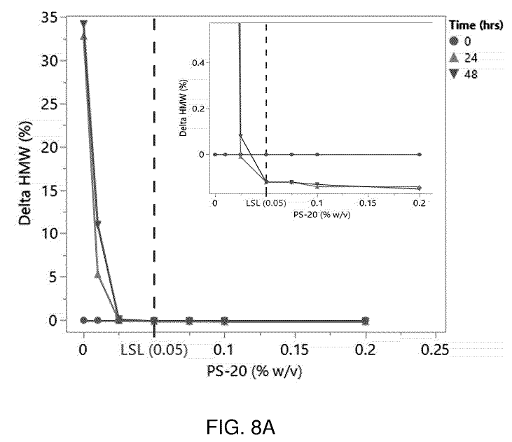

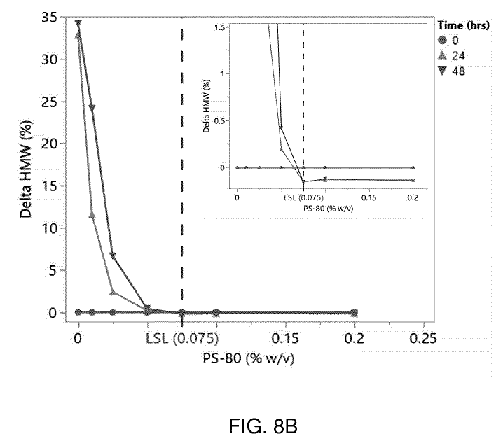

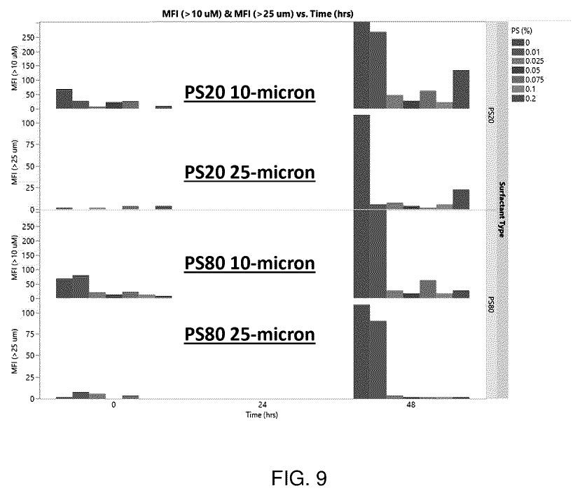

REGN1908 and REGN1909 were co-formulated in the absence or presence of a polysorbate surfactant to evaluate effects on stability. Starting with a base formulation containing a target total antibody concentration of 150 mg/mL REGN1908-REGN1909 (about 75 mg/mL each antibody), 10 mM histidine, and 70 mM arginine hydrochloride at pH 6.0, either no polysorbate was added, or polysorbate 20 or polysorbate 80 was added at an amount of 0.01%, 0.025%, 0.05%, 0.075%, 0.1%, or 0.2% (all percentages w/v). The formulations were subjected to agitation stress induced by orbital shaking at 250 rpm for 24 or 48 hours. As shown in FIG. 8, formulations containing 0.05% w/v polysorbate 20 were protected from aggregation formation (FIG. 8A), while formulations containing 0.075% w/v polysorbate 80 were protected from aggregation formation (FIG. 8B). Additionally, the presence of subvisible particles was determined using micro fluid imaging (MFI) at >10 micron and >25 micron for 48-hours samples. As shown in FIG. 9, both polysorbate 20 and polysorbate 80 were effective at ≥0.025% w/v; however, polysorbate 80 resulted in lower particle counts in both the 10-micron and 25-micron bins compared to polysorbate 20.

Co-formulations containing polysorbate 20 or polysorbate 80 and further comprising a cryoprotectant were generated and tested for stability under accelerated stability storage conditions (40° C. at 75% relative humidity) and under repeated freeze-thaw conditions. Starting with a base formulation containing a target total antibody concentration of 150 mg/mL REGN1908-REGN1909 (about 75 mg/mL each antibody), 10 mM histidine, 70 mM arginine hydrochloride, and 0.15% (w/v) polysorbate at pH 6.0, either no cryoprotectant was added, or 5% sucrose, 3% proline, 5% trehalose, or 2.5% sorbitol was added (all percentages w/v).

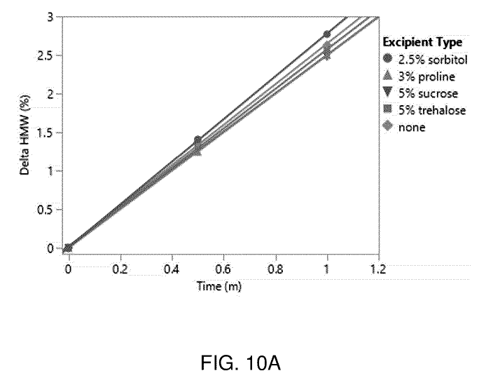

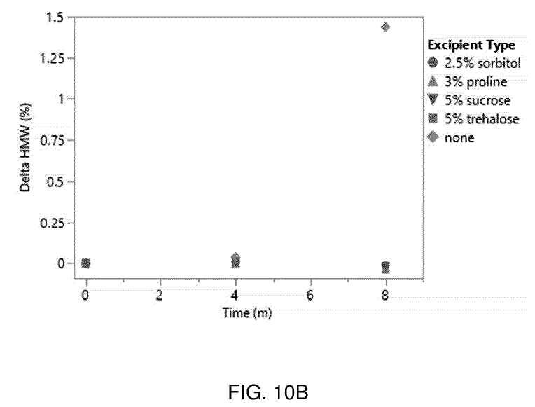

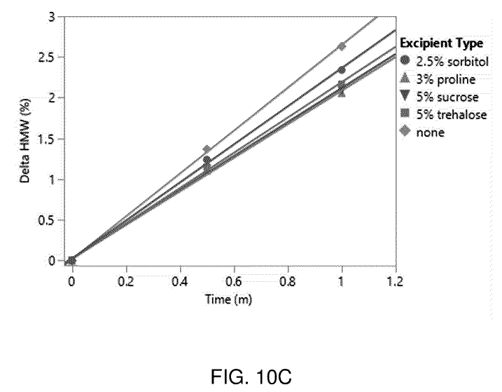

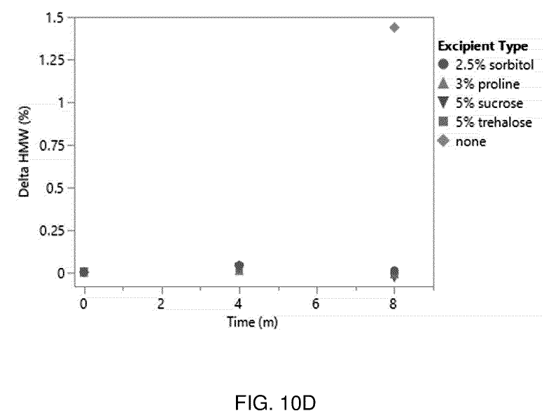

FIGS. 10A-10B show stability data for co-formulations containing polysorbate 20, while FIGS. 10C-10D show stability data for co-formulations containing polysorbate 80. FIGS. 10A and 10C show that the aggregation formation rates (slopes) for cryoprotectants were higher in the presence of polysorbate 20 than for polysorbate 80, suggesting a better stability profile for polysorbate 80 formulations. As shown in FIGS. 10B and 10D, the presence of cryoprotectant protected against HMW formation after 8 freeze/thaw cycles, for both the polysorbate 20- and polysorbate 80-containing formulations.

### Example 8: Co-Formulation Characterization (DoE)

Design of experiments (DoE) analysis was conducted on an exemplary formulation's components, scaled over a wider range than typical manufacturing specifications. Table 24 below shows the factors and ranges that were investigated. REGN1908 and REGN1909 were co-formulated at a ratio of about 1:1.

TABLE 24
Factors Investigated by DoE

| | Descrip- | | | Lower | Upper |
|---|---|---|---|---|---|
| Factor | tion | Target | Range | Level | Level |
| REGN1908- | Protein | 150 | ±10% | 135 | 165 |
| REGN1909 | | | | | |
| Concentration | | | | | |
| (mg/mL) | | | | | |
| pH | pH | 6 | ±0.3 | 5.7 | 6.3 |
| Histidine (mM) | Buffer | 10 | ±40% | 6 | 14 |
| Polysorbate 80 | Surfactant | 0.15 | 0.075-0.2 | 0.075 | 0.2 |
| (% w/v) | | | | | |
| Arginine (mM) | Viscosity | 70 | ±40% | 42 | 98 |
| | reducer | | | | |
| Sucrose (% w/v) | Thermal | 5 | ±40% | 3 | 7 |
| | stabilizer | | | | |

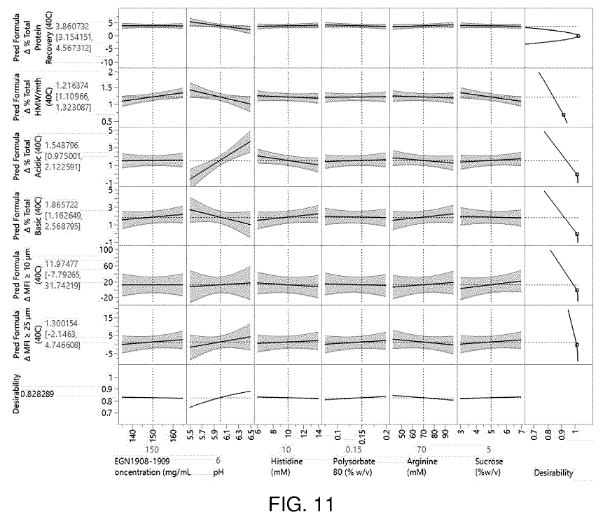

FIG. 11 shows predicted formulation responses at 40° C. as a function of ranging formulation components. Positive or negative slopes over a component's range (x-axis) indicate a formulation's attribute response (y-axis) to that change; the vertical dotted lines indicate the chosen target value for each formulation component. Most of the thermal profiler's trends are flat, indicating that protein and formulation are thermally stable over the functional range of the components. Most responses (y-axis) are minimally affected, except as a function of pH, indicating that the protein's stability is affected by the pH of the formulation.

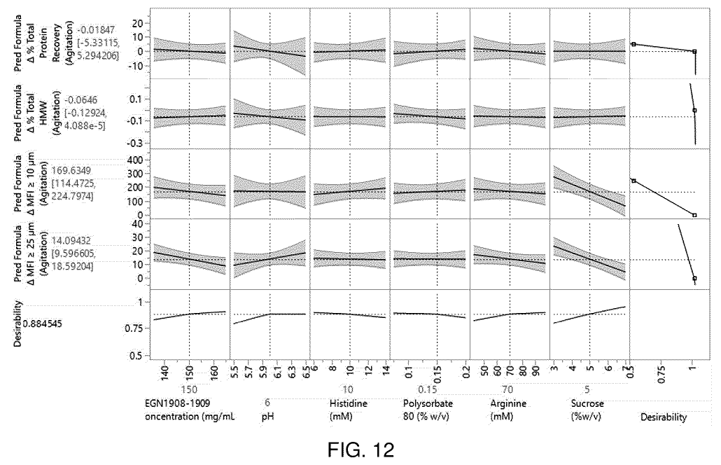

FIG. 12 shows predicted formulation responses under agitation as a function of ranging formulation components. Most trends in the agitation profiler are mostly flat across the range, indicating that protein and formulation are thermally stable over the functional range of the components. However, meaningful decreases in particulate formation are predicted with an increase of sucrose concentration.

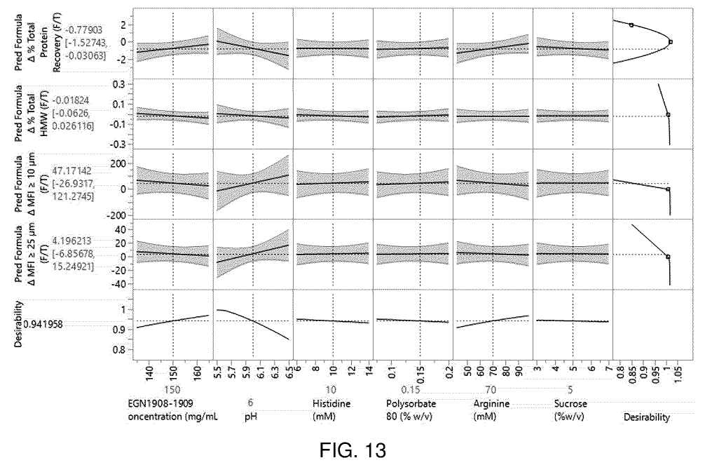

FIG. 13 shows predicted formulation responses under repeated freeze-thaw cycles as a function of ranging formulation components. Most trends in the freeze-thaw profiler are mostly flat across the range, indicating that protein and formulation are stable to freeze-thaw stress over the functional range of the components. Meaningful increases in particulate formation are predicted with an increase in pH, although particulate counts are very low at the target pH 6.0.

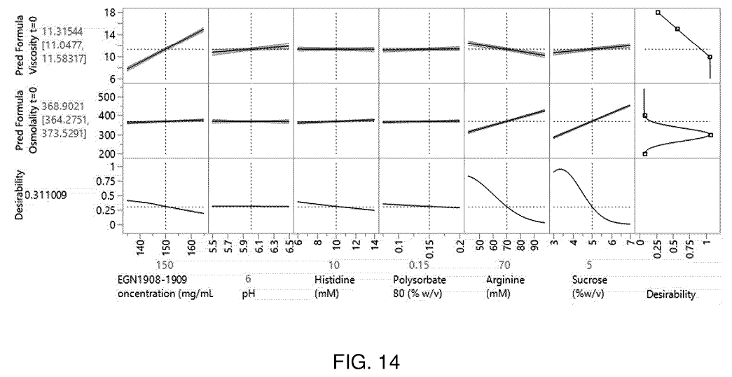

FIG. 14 shows predicted osmolality and viscosity responses as a function of ranging formulation components. Most trends in the t=0 profiler are flat across the range, indicating that protein formulations do not experience significant changes in viscosity and osmolality over the functional range of components. For viscosity, a meaningful increase in viscosity is predicted with an increase in protein concentration, and a meaningful decrease in viscosity is predicted with an increase in the viscosity reducer (arginine). For osmolality, a meaningful increase in osmolality is predicted with an increase in arginine and sucrose concentrations, although these predicted changes do not go outside the manufacturing range or beyond physiological tolerability.

### Example 9: Stability Studies for REGN1908-REGN1909 Co-Formulations

In certain embodiments, two anti-Fel d1 antibodies are co-formulated in an aqueous buffered formulation comprising: (a) from 5 mM to 20 mM histidine buffer, (b) from 2.5% to 7.5% w/v sucrose, (c) from 60 mM to 130 mM arginine, (d) from 0.01% to 0.5% w/v polysorbate, and (e) from 50 to 200 mg total anti-Fel d1 antibody, at pH 6.0±0.3.

In one exemplary embodiment, an anti-Fel d1 antibody formulation comprises: (a) 10 mM±2 mM histidine, (b) 5%±1% w/v sucrose, (c) 70 mM±14 mM arginine, (d) 0.15%±0.075% w/v polysorbate 80, and (e) 150 mg/mL±15 mg/mL REGN1908-REGN1909 (75 mg/mL REGN1908+75 mg/mL REGN1909), at pH 6.0±0.3.

REGN1908 and REGN1909 were co-formulated at a concentration of ˜150 mg/mL total antibody (about a 1:1 ratio of REGN1908:REGN1909, with about 75 mg/mL REGN1908 and about 75 mg/mL REGN1909) in 10 mM histidine, 70 mM arginine-HCl, 5% (w/v) sucrose, and 0.15% (w/v) super refined polysorbate 80 at pH 6.0 for various long-term stability studies. Stability studies on the REGN1908-1909 co-formulation were carried out in glass vials and in pre-filled syringes (PFS).

As shown in Tables 25 and 26 below, research stability studies indicated that the REGN1908-1909 co-formulation was physically and chemically stable when incubated at 5° C. for up to 24 months when stored in glass vials or in PFS, with no meaningful changes observed in any monitored stability attributes. Vial and PFS stability were comparable.

Following incubation at 40° C./75% RH for 3 months, a 10.9% increase in HMW species was observed by SE-UPLC for REGN1908-1909 co-formulation stored in glass vials, and a 9.8% increase in HMW species was observed for co-formulation stored in PFS. See Tables 27 and 28.

REGN1908-1909 co-formulation was physically and chemically stable under agitation stress (see Tables 29 and 30) and when subjected to 4 freezing and thawing cycles (Table 29). No appreciable change in the physical or chemical stability was detected in any of the monitored attributes.

TABLE 25
Stability of REGN1908-1909 Co-Formulation
Vial Drug Product at 5° C.

| | | Length of storage (months) | | |
|---|---|---|---|---|
| Assay | | 0 | 12 | 24 |
| Visual appearance (pass as clear, | | Pass | Pass | Pass |
| colorless to pale yellow, essentially free | | | | |
| from visible particles) | | | | |
| pH | | 6.0 | 6.0 | 5.9 |
| Total protein | | 155 | 157 | 151 |
| (mg/mL) | | | | |
| Purity by SE-UPLC | % main | 98.2 | 97.6 | 97.5 |
| | % LMW species | 0.0 | 0.0 | 0.0 |
| | % HMW species | 1.8 | 2.4 | 2.5 |
| Charge variant | % R1909 Main | 28.4 | 29.8 | 27.0 |
| analysis by iCIEF | (A + B) | | | |
| | % R1908 Main | 21.1 | 23.2 | 20.3 |
| Particulate matter | ≥2 to <10 μm | 1093 | 716 | 1266 |
| (MFI) - unfiltered | (particle/mL) | | | |
| | ≥10 μm | 105 | 15 | 28 |
| | (particle/mL) | | | |
| | ≥25 μm | 3 | 7 | 3 |
| | (particle/mL) | | | |
| Light Obscuration, | ≥10 μm (# particle/ | 64 | 64 | 24 |
| HIAC | container), <6000 | | | |
| | particles/container | | | |
| | ≥25 μm (# particle/ | 1 | 0 | 1 |
| | container), <600 | | | |
| | particles/container | | | |

TABLE 26
Stability of REGN1908-1909 Co-Formulation
PFS Drug Product at 5° C.

| | | Length of storage (months) | | |
|---|---|---|---|---|
| Assay | | 0 | 12 | 24 |
| Visual appearance (pass as clear, | | Pass | Pass | Pass |
| colorless to pale yellow, essentially free | | | | |
| from visible particles) | | | | |
| pH | | 6.0 | 6.0 | 6.0 |
| Total protein | | 155 | 158 | 156 |
| (mg/mL) | | | | |
| Purity by SE-UPLC | % main | 98.2 | 97.6 | 97.5 |
| | % LMW species | 0.0 | 0.0 | 0.0 |
| | % HMW species | 1.8 | 2.4 | 2.5 |
| Charge variant | % R1909 Main | 28.3 | 30.4 | 28.0 |
| analysis by iCIEF | (A + B) | | | |
| | % R1908 Main | 20.7 | 23.5 | 20.4 |
| Particulate matter | ≥2 to <10 μm | 4020 | 12201 | 3216 |
| (MFI) - unfiltered | (particle/mL) | | | |
| | ≥10 μm | 177 | 25 | 1333 |
| | (particle/mL) | | | |
| | ≥25 μm | 0 | 2 | 6 |
| | (particle/mL) | | | |
| Particulate matter | ≥2 to <10 μm | 1464 | 10054 | 1956 |
| (MFI) - filtered | (particle/mL) | | | |
| (Aspect Ratio | ≥10 μm | 15 | 8 | 21 |
| AR < 0.85) | (particle/mL) | | | |
| | ≥25 μm | 0 | 2 | 4 |
| | (particle/mL) | | | |
| Light Obscuration, | ≥10 μm (# particle/ | 29 | 127 | 96 |
| HIAC | container), <6000 | | | |
| | particles/container | | | |
| | ≥25 μm (# particle/ | 0 | 3 | 3 |
| | container), <600 | | | |
| | particles/container | | | |

TABLE 27
Stability of REGN1908-1909 Co-Formulation Vial Drug Product
at 25° C./60% RH and 40° C./75% RH

| | | | 25° C./ | 40° C./ |
|---|---|---|---|---|
| | | | 60% RH | 75% RH |
| | | No | storage | storage |
| Storage Condition | | storage | (months) | (months) |
| Assay | | 0 | 6 | 3 |
| Visual appearance (pass as clear, | | Pass | Pass | Pass |
| colorless to pale yellow, essentially | | | | |
| free from visible particles) | | | | |
| pH | | 6.0 | 6.0 | 6.0 |
| Total protein | | 155 | 164 | 148 |
| (mg/mL) | | | | |
| Purity by SE- | % main | 98.2 | 96.9 | 88.8 |
| UPLC | % LMW species | 0.0 | 0.1 | 0.3 |
| | % HMW species | 1.8 | 3.1 | 10.9 |
| Charge variant | % R1909 Main | 28.4 | 23.6 | 14.2 |
| analysis by iCIEF | (A + B) | | | |
| | % R1908 Main | 21.1 | 20.3 | 9.4 |
| Particulate matter | ≥2 to <10 μm | 1093 | 2231 | 1562 |
| (MFI) - unfiltered | (particle/mL) | | | |
| | ≥10 μm | 104 | 46 | 33 |
| | (particle/mL) | | | |
| | ≥25 μm | 2 | 6 | 8 |
| | (particle/mL) | | | |

TABLE 28
Stability of REGN1908-1909 Co-Formulation PFS Drug Product
at 25° C./60% RH and 40° C./75% RH

| | | | 25° C./ | 40° C./ |
|---|---|---|---|---|
| | | | 60% RH | 75% RH |
| | | No | storage | storage |
| Storage Condition | | storage | (months) | (months) |
| Assay | | 0 | 6 | 3 |
| Visual appearance (pass as clear, | | Pass | Pass | Pass |
| colorless to pale yellow, essentially | | | | |
| free from visible particles) | | | | |
| pH | | 6.0 | 6.0 | 6.0 |
| Total protein | | 155 | 157 | 151 |
| (mg/mL) | | | | |
| Purity by SE- | % main | 98.2 | 96.9 | 90.0 |
| UPLC | % LMW species | 0.0 | 0.1 | 0.2 |
| | % HMW species | 1.8 | 3.1 | 9.8 |
| Charge variant | % R1909 Main | 28.4 | 23.7 | 14.3 |
| analysis by iCIEF | (A + B) | | | |
| | % R1908 Main | 21.1 | 20.1 | 9.7 |
| Particulate matter | ≥2 to <10 μm | 1093 | 4099 | 22725 |
| (MFI) - unfiltered | (particle/mL) | | | |
| | ≥10 μm | 104 | 81 | 621 |
| | (particle/mL) | | | |
| | ≥25 μm | 2 | 2 | 21 |
| | (particle/mL) | | | |

TABLE 29
Stability of REGN1908-1909 Co-Formulation
Vial Drug Product Agitation and Freeze/Thaw

| | | | Orbital | Freeze/ |
|---|---|---|---|---|
| | | No | Shakers | Thaw |
| Storage Condition | | storage | (hrs) | Cycles |
| Assay | | 0 | 48 | 4 |
| Visual appearance (pass as clear, | | Pass | Pass | Pass |
| colorless to pale yellow, essentially | | | | |
| free from visible particles) | | | | |
| pH | | 6.0 | 6.0 | 6.0 |
| Total protein | | 155 | 159 | 151 |
| (mg/mL) | | | | |
| Purity by SE- | % main | 98.2 | 98.2 | 98.0 |
| UPLC | % LMW species | 0.0 | 0.0 | 0.0 |
| | % HMW species | 1.8 | 1.8 | 2.0 |
| Charge variant | % R1909 Main | 28.4 | 28.5 | 28.2 |
| analysis by iCIEF | (A + B) | | | |
| | % R1908 Main | 21.1 | 21.4 | 20.8 |
| Particulate matter | ≥2 to <10 μm | 1093 | 1437 | 940 |
| (MFI) - unfiltered | (particle/mL) | | | |
| | ≥10 μm | 104 | 90 | 63 |
| | (particle/mL) | | | |
| | ≥25 μm | 2 | 6 | 2 |
| | (particle/mL) | | | |

TABLE 30
Stability of REGN1908-1909 Co-Formulation
PFS Drug Product Agitation

| Storage Condition | | No storage | Tumbler (hrs) |
|---|---|---|---|
| Assay | | 0 | 48 |
| Visual appearance (pass as clear, | | Pass | Pass |
| colorless to pale yellow, essentially free | | | |
| from visible particles) | | | |
| pH | | 6.0 | 6.1 |
| Total protein (mg/mL) | | 155 | 153 |
| Purity by SE-UPLC | % main | 98.1 | 98.1 |
| | % LMW species | 0.0 | 0.0 |
| | % HMW species | 1.9 | 1.9 |
| Charge variant | % R1909 Main | 28.3 | 28.5 |
| analysis by iCIEF | (A + B) | | |
| | % R1908 Main | 20.7 | 21.3 |
| Particulate matter | ≥2 to <10 μm | 3181 | 3087 |
| (MFI) unfiltered | (particle/mL) | | |
| | ≥10 μm | 86 | 81 |
| | (particle/mL) | | |
| | ≥25 μm | 0 | 8 |
| | (particle/mL) | | |

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. The disclosures of all patents and non-patent literature cited herein are expressly incorporated in their entirety by reference.

What is claimed is:

1. A stable liquid pharmaceutical formulation comprising:
(a) at least one antibody that specifically binds to Fel d1, wherein the antibody is selected from the group consisting of:
(i) an antibody comprising an HCDR1 comprising the amino acid sequence of SEQ ID NO:4, an HCDR2 comprising the amino acid sequence of SEQ ID NO:6, an HCDR3 comprising the amino acid sequence of SEQ ID NO:8, an LCDR1 comprising the amino acid sequence of SEQ ID NO:12, an LCDR2 comprising the amino acid sequence AAS, and an LCDR3 comprising the amino acid sequence of SEQ ID NO:14; and
(ii) an antibody comprising an HCDR1 comprising the amino acid sequence of SEQ ID NO:18, an HCDR2 comprising the amino acid sequence of SEQ ID NO:20, an HCDR3 comprising the amino acid sequence of SEQ ID NO:22, an LCDR1 comprising the amino acid sequence of SEQ ID NO:26, an LCDR2 comprising the amino acid sequence KAS, and an LCDR3 comprising the amino acid sequence of SEQ ID NO:28;
(b) a buffer comprising histidine;
(c) a thermal stabilizer; and
(d) a surfactant;
wherein the formulation has a pH of 6.0±0.3.

2. The stable liquid pharmaceutical formulation of claim 1, comprising (i) a first antibody that specifically binds to Fel d1, wherein the first antibody comprises an HCDR1 comprising the amino acid sequence of SEQ ID NO:4, an HCDR2 comprising the amino acid sequence of SEQ ID NO:6, an HCDR3 comprising the amino acid sequence of SEQ ID NO:8, an LCDR1 comprising the amino acid sequence of SEQ ID NO:12, an LCDR2 comprising the amino acid sequence AAS, and an LCDR3 comprising the amino acid sequence of SEQ ID NO:14; and (ii) a second antibody that specifically binds to Fel d1, wherein the second antibody comprises an HCDR1 comprising the amino acid sequence of SEQ ID NO:18, an HCDR2 comprising the amino acid sequence of SEQ ID NO:20, an HCDR3 comprising the amino acid sequence of SEQ ID NO:22, an LCDR1 comprising the amino acid sequence of SEQ ID NO:26, an LCDR2 comprising the amino acid sequence KAS, and an LCDR3 comprising the amino acid sequence of SEQ ID NO:28.

3. The stable liquid pharmaceutical formulation of claim 2, wherein the first antibody comprises a heavy chain variable region (HCVR) comprising the amino acid sequence of SEQ ID NO:2 and a light chain variable region (LCVR) comprising the amino acid sequence of SEQ ID NO:10.

4. The stable liquid pharmaceutical formulation of claim 2, wherein the second antibody comprises an HCVR comprising the amino acid sequence of SEQ ID NO:16 and an LCVR comprising the amino acid sequence of SEQ ID NO:24.

5. The stable liquid pharmaceutical formulation of claim 1, wherein the total antibody concentration is from 5 mg/mL to 200 mg/mL.

6. The stable liquid pharmaceutical formulation of claim 5, wherein the total antibody concentration is 150 mg/mL±15 mg/mL.

7. The stable liquid pharmaceutical formulation of claim 2, wherein the first antibody and the second antibody are present in the formulation at a molecular ratio of about 1:1.

8. The stable liquid pharmaceutical formulation of claim 2, wherein the first antibody is present at a concentration of 75 mg/mL±7.5 mg/mL and the second antibody is present at a concentration of 75 mg/mL±7.5 mg/mL.

9. The stable liquid pharmaceutical formulation of claim 1, wherein the buffer comprises histidine at a concentration of from 5 mM to 25 mM.

10. The stable liquid pharmaceutical formulation of claim 9, wherein the histidine is present at a concentration of 10 mM±2 mM.

11. The stable liquid pharmaceutical formulation of claim 1, wherein the thermal stabilizer comprises sucrose, trehalose, proline, or sorbitol.

12. The stable liquid pharmaceutical formulation of claim 11, wherein the thermal stabilizer comprises sucrose at a concentration of from 2.5% w/v to 7.5% w/v.

13. The stable liquid pharmaceutical formulation of claim 12, wherein the sucrose is present at a concentration of 5% w/v±1% w/v.

14. The stable liquid pharmaceutical formulation of claim 1, further comprising a viscosity reducer.

15. The stable liquid pharmaceutical formulation of claim 14, wherein the viscosity reducer comprises arginine.

16. The stable liquid pharmaceutical formulation of claim 15, wherein the arginine is present at a concentration of 60 mM to 130 mM.

17. The stable liquid pharmaceutical formulation of claim 15, wherein the arginine is present at a concentration of at least 70 mM.

18. The stable liquid pharmaceutical formulation of claim 15, wherein the arginine is present at a concentration of 70 mM±14 mM.

19. The stable liquid pharmaceutical formulation of claim 1, wherein the surfactant comprises polysorbate 20 or polysorbate 80.

20. The stable liquid pharmaceutical formulation of claim 19, wherein the polysorbate 20 or polysorbate 80 is present at a concentration of from 0.05% w/v to 0.2% w/v.

21. The stable liquid pharmaceutical formulation of claim 19, wherein the surfactant comprises polysorbate 80 at a concentration of 0.15% w/v±0.075% w/v.

22. The stable liquid pharmaceutical formulation of claim 1, comprising:
histidine at a concentration of 10 mM±2 mM;
sucrose at a concentration of 5% w/v±1% w/v;
arginine at a concentration of 70 mM±14 mM;
polysorbate 80 at a concentration of 0.15% w/v±0.075% w/v;
wherein the total antibody concentration in the formulation is 150 mg/mL±15 mg/mL, and wherein the formulation has a pH 6.0±0.3.

23. The stable liquid pharmaceutical formulation of claim 1, wherein the formulation has a viscosity ≤25 cP at 20° C.

24. The stable liquid pharmaceutical formulation of claim 23, wherein the formulation has a viscosity ≤15 cP at 20° C.

25. The stable liquid pharmaceutical formulation of claim 1, wherein the formulation comprises:
an anti-Fel d1 antibody that comprises an HCVR comprising the amino acid sequence of SEQ ID NO:2 and an LCVR comprising the amino acid sequence of SEQ ID NO:10, wherein the concentration of the antibody is from 50 mg/mL to 190 mg/mL;
histidine at a concentration of 10 mM±2 mM;
sucrose at a concentration of 5% w/v±1% w/v;
arginine at a concentration of 70 mM±14 mM; and
polysorbate 80 at a concentration of 0.15% w/v±0.075% w/v;
wherein the formulation has a pH 6.0±0.3.

26. The stable liquid pharmaceutical formulation of claim 1, wherein the formulation comprises:
an anti-Fel d1 antibody that comprises an HCVR comprising the amino acid sequence of SEQ ID NO:16 and an LCVR comprising the amino acid sequence of SEQ ID NO:24, wherein the concentration of the antibody is from 50 mg/mL to 190 mg/mL;
histidine at a concentration of 10 mM±2 mM;
sucrose at a concentration of 5% w/v±1% w/v; and
polysorbate 20 or polysorbate 80 at a concentration of 0.15% w/v±0.075% w/v;
wherein the formulation has a pH 6.0±0.3.

27. The stable liquid pharmaceutical formulation of claim 26, further comprising a viscosity reducer.

28. The stable liquid pharmaceutical formulation of claim 27, wherein the viscosity reducer is arginine at a concentration of 70 mM±14 mM.

29. The stable liquid pharmaceutical formulation of claim 1, wherein the formulation comprises:
a first antibody that specifically binds to Fel d1, wherein the first antibody comprises an HCVR comprising the amino acid sequence of SEQ ID NO:2 and an LCVR comprising the amino acid sequence of SEQ ID NO:10, wherein the concentration of the first antibody is 75 mg/mL±10 mg/mL;
a second antibody that specifically binds to Fel d1, wherein the second antibody comprises an HCVR comprising the amino acid sequence of SEQ ID NO:16 and an LCVR comprising the amino acid sequence of SEQ ID NO:24, wherein the concentration of the second antibody is 75 mg/mL±10 mg/mL;
histidine at a concentration of 10 mM±2 mM;
sucrose at a concentration of 5% w/v±1% w/v;
arginine at a concentration of 70 mM±14 mM; and
polysorbate 80 at a concentration of 0.15% w/v±0.075% w/v;
wherein the formulation has a pH 6.0±0.3.

30. A stable liquid pharmaceutical formulation comprising:
(a) an antibody that specifically binds to Fel d1, wherein the antibody comprises an HCDR1 comprising the amino acid sequence of SEQ ID NO:4, an HCDR2 comprising the amino acid sequence of SEQ ID NO:6, an HCDR3 comprising the amino acid sequence of SEQ ID NO:8, an LCDR1 comprising the amino acid sequence of SEQ ID NO:12, an LCDR2 comprising the amino acid sequence AAS, and an LCDR3 comprising the amino acid sequence of SEQ ID NO:14;
(b) a buffer comprising histidine;
(c) a thermal stabilizer; and
(d) a surfactant;
wherein the formulation has a pH of 6.0±0.3.

31. The stable liquid pharmaceutical formulation of claim 30, wherein the antibody comprises an HCVR comprising the amino acid sequence of SEQ ID NO:2 and an LCVR comprising the amino acid sequence of SEQ ID NO:10.

32. The stable liquid pharmaceutical formulation of claim 30, wherein the formulation comprises:
an anti-Fel d1 antibody that comprises an HCVR comprising the amino acid sequence of SEQ ID NO:2 and an LCVR comprising the amino acid sequence of SEQ ID NO:10, wherein the concentration of the antibody is from 50 mg/mL to 190 mg/mL;
histidine at a concentration of 10 mM±2 mM;
sucrose at a concentration of 5% w/v±1% w/v; and
polysorbate 80 at a concentration of 0.15% w/v±0.075% w/v;
wherein the formulation has a pH 6.0±0.3.

33. A stable liquid pharmaceutical formulation comprising:
(a) an antibody that specifically binds to Fel d1, wherein the antibody comprises an HCDR1 comprising the amino acid sequence of SEQ ID NO:18, an HCDR2 comprising the amino acid sequence of SEQ ID NO:20, an HCDR3 comprising the amino acid sequence of SEQ ID NO:22, an LCDR1 comprising the amino acid sequence of SEQ ID NO:26, an LCDR2 comprising the amino acid sequence KAS, and an LCDR3 comprising the amino acid sequence of SEQ ID NO:28;
(b) a buffer comprising histidine;
(c) a thermal stabilizer; and
(d) a surfactant;
wherein the formulation has a pH of 6.0±0.3.

34. The stable liquid pharmaceutical formulation of claim 33, wherein the antibody comprises an HCVR comprising the amino acid sequence of SEQ ID NO:16 and an LCVR comprising the amino acid sequence of SEQ ID NO:24.

35. The stable liquid pharmaceutical formulation of claim 33, wherein the formulation comprises:
an anti-Fel d1 antibody that comprises an HCVR comprising the amino acid sequence of SEQ ID NO:16 and an LCVR comprising the amino acid sequence of SEQ ID NO: 24, wherein the concentration of the antibody is from 50 mg/mL to 190 mg/mL;
histidine at a concentration of 10 mM±2 mM;
sucrose at a concentration of 5% w/v±1% w/v; and
polysorbate 20 or polysorbate 80 at a concentration of 0.15% w/v±0.075% w/v;
wherein the formulation has a pH 6.0±0.3.

36. The stable liquid pharmaceutical formulation of claim 35, further comprising a viscosity reducer.

37. The stable liquid pharmaceutical formulation of claim 36, wherein the viscosity reducer is arginine at a concentration of 70 mM±14 mM.

38. The stable liquid pharmaceutical formulation of claim 1, contained in a container that is selected from the group consisting of a glass vial, a syringe, a pre-filled syringe, a pen delivery device, and an autoinjector delivery device.

39. The stable liquid pharmaceutical formulation of claim 1, contained in a large volume device or bolus injector.

40. A container containing the stable liquid pharmaceutical formulation of claim 1.

41. A kit comprising:
(i) a container containing the stable liquid pharmaceutical formulation of claim 1; and
(ii) labeling for use of the pharmaceutical formulation.

42. The kit of claim 41, wherein the labeling recites subcutaneous or intravenous administration of the pharmaceutical formulation.


SEQUENCE LISTING (32 sequences)

SEQ ID NO: 1 - DNA, 357 bp, synthetic construct
  source 1..357: mol_type = other DNA
gaggtgcagctggtggagtctgggggaggcctggtcaagcctggggggtccctgagactctcctgtgcagcctctggattcaccttcagaaactataacataaactgggtccgccaggctccagggaaggggctggagtgggtctcactcatcagtggtagtagtagttacatatattacgcagactcagtgaagggccgattcaccgtctccagagacaacgccaagaactcactgtatctgcaaatgaacagcctgagagccgaggacacggctgtgtattactgtgcgaggcggacattaagctactacgttatggacgtctggggccaagggaccacggtcaccgtctcctca

SEQ ID NO: 2 - protein, 119 aa, synthetic construct
EVQLVESGGGLVKPGGSLRLSCAASGFTFRNYNINWVRQAPGKGLEWVSLISGSSSYIYYADSVKGRFTVSRDNAKNSLYLQMNSLRAEDTAVYYCARRTLSYYVMDVWGQGTTVTVSS

SEQ ID NO: 3 - DNA, 24 bp, synthetic construct
  source 1..24: mol_type = other DNA
ggattcaccttcagaaactataac

SEQ ID NO: 4 - protein, 8 aa, synthetic construct
GFTFRNYN

SEQ ID NO: 5 - DNA, 24 bp, synthetic construct
  source 1..24: mol_type = other DNA
atcagtggtagtagtagttacata

SEQ ID NO: 6 - protein, 8 aa, synthetic construct
ISGSSSYI

SEQ ID NO: 7 - DNA, 36 bp, synthetic construct
  source 1..36: mol_type = other DNA
gcgaggcggacattaagctactacgttatggacgtc

SEQ ID NO: 8 - protein, 12 aa, synthetic construct
ARRTLSYYVMDV

SEQ ID NO: 9 - DNA, 321 bp, synthetic construct
  source 1..321: mol_type = other DNA
gacatccaggtgacccagtctccatcccccctgtctgcatctgtaggagacagagtcaccatcacttgccgggcgagtcagggcattagcaattatttagcctggtatcagcagaaaccagggagagttcctcagctcctgatctatgctgcatccactttgcaatcaggggtcccatctcggttcagtggcagtggatctgggacagatttcactctcaccatcagcagcctgcagcctgaagatgttgcaacttattactgtcaaaagtataacagtgccccgtacacttttggccaggggaccaagctggagatcaaa

SEQ ID NO: 10 - protein, 107 aa, synthetic construct
DIQVTQSPSPLSASVGDRVTITCRASQGISNYLAWYQQKPGRVPQLLIYAASTLQSGVPSRFSGSGSGTDFTLTISSLQPEDVATYYCQKYNSAPYTFGQGTKLEIK

SEQ ID NO: 11 - DNA, 18 bp, synthetic construct
  source 1..18: mol_type = other DNA
cagggcattagcaattat

SEQ ID NO: 12 - protein, 6 aa, synthetic construct
QGISNY

SEQ ID NO: 13 - DNA, 27 bp, synthetic construct
  source 1..27: mol_type = other DNA
caaaagtataacagtgccccgtacact

SEQ ID NO: 14 - protein, 9 aa, synthetic construct
QKYNSAPYT

SEQ ID NO: 15 - DNA, 345 bp, synthetic construct
  source 1..345: mol_type = other DNA
gaggtgcagctggtggagtctgggggaggcttggtacagcctggggggtccctgagactctcctgtgcagcctctggattcacctttagcagttatgccatgagctgggtccgccaggctccagggaaggggctggagtgggtctcagctattagtggtcgtggttataacgcagactacgcagactccgtgaagggccggttcaccatctccagggacaattccaagaacacgctgtatctgcaaatgaacagcctgagagccgaagacacggccgtatattactgtgcgaaattggaatactttgactactggggccagggaaccctggtcactgtctcctca

SEQ ID NO: 16 - protein, 115 aa, synthetic construct
EVQLVESGGGLVQPGGSLRLSCAASGFTFSSYAMSWVRQAPGKGLEWVSAISGRGYNADYADSVKGRFTISRDNSKNTLYLQMNSLRAEDTAVYYCAKLEYFDYWGQGTLVTVSS

SEQ ID NO: 17 - DNA, 24 bp, synthetic construct
  source 1..24: mol_type = other DNA
ggattcacctttagcagttatgcc

SEQ ID NO: 18 - protein, 8 aa, synthetic construct
GFTFSSYA

SEQ ID NO: 19 - DNA, 24 bp, synthetic construct
  source 1..24: mol_type = other DNA
attagtggtcgtggttataacgca

SEQ ID NO: 20 - protein, 8 aa, synthetic construct
ISGRGYNA

SEQ ID NO: 21 - DNA, 24 bp, synthetic construct
  source 1..24: mol_type = other DNA
gcgaaattggaatactttgactac

SEQ ID NO: 22 - protein, 8 aa, synthetic construct
AKLEYFDY

SEQ ID NO: 23 - DNA, 321 bp, synthetic construct
  source 1..321: mol_type = other DNA
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagatttcactctcaccatcagcagcctgaggcctgaagattttgcaacttattactgccaacagtataatagttaccctctgactttcggcggagggaccaaggtggagatcaaa

SEQ ID NO: 24 - protein, 107 aa, synthetic construct
DIQMTQSPSTLSASVGDRVTITCRASQSISSWLAWYQQKPGKAPKLLIYKASSLESGVPSRFSGSGSGTDFTLTISSLRPEDFATYYCQQYNSYPLTFGGGTKVEIK

SEQ ID NO: 25 - DNA, 18 bp, synthetic construct
  source 1..18: mol_type = other DNA
cagagtattagtagctgg

SEQ ID NO: 26 - protein, 6 aa, synthetic construct
QSISSW

SEQ ID NO: 27 - DNA, 27 bp, synthetic construct
  source 1..27: mol_type = other DNA
caacagtataatagttaccctctgact

SEQ ID NO: 28 - protein, 9 aa, synthetic construct
QQYNSYPLT

SEQ ID NO: 29 - protein, 446 aa, synthetic construct
EVQLVESGGGLVKPGGSLRLSCAASGFTFRNYNINWVRQAPGKGLEWVSLISGSSSYIYYADSVKGRFTVSRDNAKNSLYLQMNSLRAEDTAVYYCARRTLSYYVMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 214 aa, synthetic construct
DIQVTQSPSPLSASVGDRVTITCRASQGISNYLAWYQQKPGRVPQLLIYAASTLQSGVPSRFSGSGSGTDFTLTISSLQPEDVATYYCQKYNSAPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 442 aa, synthetic construct
EVQLVESGGGLVQPGGSLRLSCAASGFTFSSYAMSWVRQAPGKGLEWVSAISGRGYNADYADSVKGRFTISRDNSKNTLYLQMNSLRAEDTAVYYCAKLEYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 214 aa, synthetic construct
DIQMTQSPSTLSASVGDRVTITCRASQSISSWLAWYQQKPGKAPKLLIYKASSLESGVPSRFSGSGSGTDFTLTISSLRPEDFATYYCQQYNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC